SEQUENCE LISTING (58 sequences)

SEQ ID NO: 1 - DNA, 500 bp, Homo sapiens
  source 1..500: mol_type = genomic DNA
tgaattctccatccttggactggacctctcgtctgtttataccttgctgtgcagtttgtttctcctgggcgtcatttcttcttcaatttcctcaaatcctacttgcttcttagctccttcatatcctctttgttttctttgcttctgcagaaccccagattacacccctggtctccagttctccatctccatgtctgcctgttcctttctgcatcactggctcctaactcaagcaatctccttgggtttctctgaggggcccctgggatcccctatcattagtccctctcacagaagcatacccttctccagagctaaaggatcagatattcagcggctcaggtaacaaacctgctgtcaggttacacatattgtttcctgaaagaccacactacagtgtcagtggagcctcaggttgcctgcagtgccctgccctcacctggctatcccacacaggtgagaataaccagaactcacctccggtaccagtgttcacttgg

SEQ ID NO: 2 - DNA, 537 bp, Mus musculus
  source 1..537: mol_type = genomic DNA
gccatctcttcagcaccaatactgtgggactctggatagtgtgtctaaggtaacaactctcctcttcctaaatagtctacccttgaaacatctatcttaatatcttctcgctgcccactgctggctgccagaccatttctcaaattcttcaaatctcagttgtctctcaaactccttttgtactttctttgtttgatcacatttttctgtagacccccagattatatttttagtctgccttcttctctctggctcttaactcatgccgtctccttgggtaatactctgagggccccgagatcccctgtcattggtccctctaacagaagtacacccttctcccaaagccagaggatcaggtgttcagtggctccggtgacaggtgtgatgtcaggttacagatactgtttcctgagagaccacacgacagtgtcagaggaaccccagcctgcccacaatgccccgtcttcacctggctatcccttcatggtgagcatagccagaactcacctctaggcccagtgtgcacctggaaat

SEQ ID NO: 3 - protein, 1775 aa, Homo sapiens
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 4 - protein, 1809 aa, Mus musculus
MALSLQPQLLLLLSLLPQEVTSAPTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLL

SEQ ID NO: 5 - DNA, 5328 bp, Homo sapiens
  source 1..5328: mol_type = other DNA
atggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctatga

SEQ ID NO: 6 - DNA, 5430 bp, Mus musculus
  source 1..5430: mol_type = other DNA
atggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattatga

SEQ ID NO: 7 - protein, 92 aa, synthetic construct
CLSYDTEILTVEYGFLPIGKIVEERIECTVYTVDKNGFVYTQPIAQWHNRGEQEVFEYCLEDGSIIRATKDHKFMTTDGQMLPIDEIFERGL

SEQ ID NO: 8 - protein, 35 aa, synthetic construct
VKIISRKSLGTQNVYDIGVEKDHNFLLKNGLVASN

SEQ ID NO: 9 - protein, 101 aa, synthetic construct
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 10 - protein, 36 aa, synthetic construct
MVKIISRKSLGTQNVYDIGVEKDHNFLLKNGLVASN

SEQ ID NO: 11 - protein, 35 aa, synthetic construct
VKIISRKSLGTQNVYDIGVGEPHNFLLKNGLVASN

SEQ ID NO: 12 - protein, 102 aa, synthetic construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 13 - protein, 36 aa, synthetic construct
MIKIATRKYLGKQNVYDIGVERDHNFALKNGFIASN

SEQ ID NO: 14 - protein, 101 aa, synthetic construct; identical to SEQ ID NO: 9

SEQ ID NO: 15 - protein, 53 aa, synthetic construct
MKRTADGSEFESPKKKRKVKIISRKSLGTQNVYDIGVEKDHNFLLKNGLVASN

SEQ ID NO: 16 - protein, 12 aa, synthetic construct
CFSGDTLVALTD

SEQ ID NO: 17 - protein, 11 aa, synthetic construct
CLAGDTLITLA

SEQ ID NO: 18 - protein, 10 aa, synthetic construct
CLQNGTRLLR

SEQ ID NO: 19 - protein, 11 aa, synthetic construct
CLTGDSQVLTR

SEQ ID NO: 20 - protein, 11 aa, synthetic construct
CLTYETEIMTV

SEQ ID NO: 21 - protein, 12 aa, synthetic construct
CLSGNTKVRFRY

SEQ ID NO: 22 - protein, 6 aa, synthetic construct
GVFVHN

SEQ ID NO: 23 - protein, 6 aa, synthetic construct
GLLVHN

SEQ ID NO: 24 - protein, 6 aa, synthetic construct
GLIASN

SEQ ID NO: 25 - protein, 6 aa, synthetic construct
GLVVHN

SEQ ID NO: 26 - protein, 102 aa, synthetic construct; identical to SEQ ID NO: 12

SEQ ID NO: 27 - protein, 35 aa, synthetic construct
IKIATRKYLGKQNVYDIGVERDHNFALKNGFIASN

SEQ ID NO: 28 - protein, 106 aa, synthetic construct
CLAGDTLITLADGRRVPIRELVSQQNFSVWALNPQTYRLERARVSRAFCTGIKPVYRLTTRLGRSIRATANHRFLTPQGWKRVDELQPGDYLALPRRIPTASTPTL

SEQ ID NO: 29 - protein, 51 aa, synthetic construct
AAACPELRQLAQSDVYWDPIVSIEPDGVEEVFDLTVPGPHNFVANDIIAHN

SEQ ID NO: 30 - protein, 102 aa, synthetic construct
CLSYDTEILTVEYGILPIGKIVEKRIECTVYSVDNNGNIYTQPVAQWHDRGEQEVFEYCLEDGSLIRATKDHKFMTVDGQMMPIDEIFERELDLMRVDNLPN

SEQ ID NO: 31 - protein, 35 aa, synthetic construct
VKVIGRRSLGVQRIFDIGLPQYHNFLLANGAIAAN

SEQ ID NO: 32 - protein, 101 aa, synthetic construct; identical to SEQ ID NO: 9

SEQ ID NO: 33 - protein, 35 aa, synthetic construct; identical to SEQ ID NO: 8

SEQ ID NO: 34 - protein, 101 aa, synthetic construct; identical to SEQ ID NO: 9

SEQ ID NO: 35 - protein, 52 aa, synthetic construct
KRTADGSEFESPKKKRKVKIISRKSLGTQNVYDIGVEKDHNFLLKNGLVASN

SEQ ID NO: 36 - protein, 123 aa, synthetic construct
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 37 - protein, 36 aa, synthetic construct
VKVIGRRSLGVQRIFDIGLPQDHNFLLANGAIAANC

SEQ ID NO: 38 - protein, 97 aa, synthetic construct
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLQVD

SEQ ID NO: 39 - protein, 38 aa, synthetic construct
GLPVKIISRKSLGTQNVYDIGVEKDHNFLLKNGLVASN

SEQ ID NO: 40 - DNA, 306 bp, synthetic construct
  source 1..306: mol_type = other DNA
tgcctgagctacgagaccgagatcctgaccgtggagtacggcctgctgcccatcggcaagatcgtggagaagagaatcgagtgcaccgtgtacagcgtggacaacaacggcaacatctacacccagcccgtggcccagtggcacgacagaggcgagcaggaggtgttcgagtactgcctggaggacggcagcctgatcagagccaccaaggaccacaagttcatgaccgtggacggccagatgctgcccatcgacgagatcttcgagagagagctggacctgatgagagtggacaacctgcccaac

SEQ ID NO: 41 - DNA, 105 bp, synthetic construct
  source 1..105: mol_type = other DNA
atcaagatcgccacaagaaagtacctgggcaagcagaacgtgtacgacatcggcgtggagagagaccacaacttcgccctgaagaacggcttcatcgccagcaat

SEQ ID NO: 42 - DNA, 865 bp, Homo sapiens
  source 1..865: mol_type = genomic DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 43 - DNA, 437 bp, Homo sapiens
  source 1..437: mol_type = genomic DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtga

SEQ ID NO: 44 - DNA, 428 bp, Homo sapiens
  source 1..428: mol_type = genomic DNA
gctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcaggcgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 45 - DNA, 287 bp, Homo sapiens
  source 1..287: mol_type = genomic DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 46 - DNA, 290 bp, Homo sapiens
  source 1..290: mol_type = genomic DNA
cgtataggaggaccgtgtaggccttcctgtcccgggccttgccagcggccagcccgatgaaggagctccctcgcagggggtagcctccgaaggagaagacgtgggagtggtcggcagtgacgaggctcagcgtgtcctcctcgctggtgagctggcccgccctctcaatggcgtcgtcgaacatgatcgtctcagtcagtgcccggtaagccctgctttcatgatgaccatggtcgatgcgaccaccctccacgaagaggaagaagccgcgggggtgtctgctcagcagg

SEQ ID NO: 47 - DNA, 288 bp, Homo sapiens
  source 1..288: mol_type = genomic DNA
cgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacgtaccgggcaccctggcgttcgccgagccattcctgcaccagattcttcccgtccagcctggtcccaccttggctgtagtcatctgggtactcagggtctggggttcccatgcgaaacatgtactttcggcctcca

SEQ ID NO: 48 - DNA, 564 bp, Homo sapiens
  source 1..564: mol_type = genomic DNA
ctgctcagctcttgacactctgtggttctgggtagtagttgaaagcagctctcctcttcctgaattctccatccttggactggacctctcgtctgtttataccttgctgtgcagtttgtttctcctgggcgtcatttcttcttcaatttcctcaaatcctacttgcttcttagctccttcatatcctctttgttttctttgcttctgcagaaccccagattacacccctggtctccagttctccatctccatgtctgcctgttcctttctgcatcactggctcctaactcaagcaatctccttgggtttctctgaggggcccctgggatcccctatcattagtccctctcacagaagcatacccttctccagagctaaaggatcagatattcagcggctcaggtaacaaacctgctgtcaggttacacatattgtttcctgaaagaccacactacagtgtcagtggagcctcaggttgcctgcagtgccctgccctcacctggctatcccacacaggtgagaataaccagaactcacctccggtaccagtgttcacttggaaac

SEQ ID NO: 49 - protein, 88 aa, synthetic construct
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 50 - protein, 37 aa, synthetic construct
MMLKKILKIEELDERELIDIEVSGNHLFYANDILTHN

SEQ ID NO: 51 - protein, 89 aa, synthetic construct
CLSLDTMVVTNGKAIEIRDVKVGDWLESECGPVQVTEVLPIIKQPVFEIVLKSGKKIRVSANHKFPTKDGLKTINSGLKVGDFLRSRAK

SEQ ID NO: 52 - protein, 45 aa, synthetic construct
MCEIFENEIDWDEIASIEYVGVEETIDINVTNDRLFFANGILTHN

SEQ ID NO: 53 - protein, 105 aa, synthetic construct
CLVGSSEIITRNYGKTTIKEVVEIFDNDKNIQVLAFNTHTDNIEWAPIKAAQLTRPNAELVELEINTLHGVKTIRCTPDHPVYTKNRDYVRADELTDDDELVVAI

SEQ ID NO: 54 - protein, 40 aa, synthetic construct
MEAKTYIGKLKSRKIVSNEDTYDIQTSTHNFFANDILVHN

SEQ ID NO: 55 - protein, 101 aa, synthetic construct
CFVPGTLVNTENGLKKIEEIKVGDKVFSHTGKLQEVVDTLIFDRDEEIISINGIDCTKNHEFYVIDKENANRVNEDNIHLFARWVHAEELDMKKHLLIELE

SEQ ID NO: 56 - protein, 40 aa, synthetic construct
MKFKLKEITSIETKHYKGKVHDLTVNQDHSYNVRGTVVHN

SEQ ID NO: 57 - protein, 106 aa, synthetic construct
CLTGDAKIDVLIDNIPISQISLEEVVNLFNEGKEIYVLSYNIDTKEVEYKEISDAGLISESAEVLEIIDEETGQKIVCTPDHKVYTLNRGYVSAKDLKEDDELVFS

SEQ ID NO: 58 - protein, 34 aa, synthetic construct
MGLKIIKRESKEPVFDITVKDNSNFFANNILVHN

(19) United States
(12) Patent Application Publication
(10) Pub. No.: US 2024/0401079 A1
(43) Pub. Date: Dec. 5, 2024

(54) STEREOCILIN PROMOTERS AND USES THEREOF

(71) Applicants: DECIBEL THERAPEUTICS, INC., Boston, MA (US); REGENERON PHARMACEUTICALS, INC., Tarrytown, NY (US)

(72) Inventors: Lars BECKER, Boston, MA (US); Joseph BURNS, Newton, MA (US); Tyler GIBSON, Boston, MA (US); Martin SCHWANDER, Aubumdale, MA (US); Leah SABIN, Goldens Bridge, NY (US); Meghan DRUMMOND SAMUELSON, Katonah, NY (US)

(21) Appl. No.: 18/699,454

(22) PCT Filed: Oct. 12, 2022
(86) PCT No.: PCT/US2022/046444

§ 371 Date: Apr. 8, 2024

Related U.S. Application Data
(60) Provisional application No. 63/254,930, filed on Oct. 12, 2021.
(60) Provisional application No. 63/307,484, filed on Feb. 7, 2022.
(60) Provisional application No. 63/338,605, filed on May 5, 2022.

(51) Int. Cl. C12N 15/86; A61P 27/16; C07K 14/47

(52) U.S. Cl. CPC C12N 15/86; A61P 27/16; C07K 14/47; C12N 2750/14143; C12N 2800/40

(57) ABSTRACT

The disclosure provides stereocilin (STRC) promoters, as well as vectors containing the same, that can be used to express a desired expression product in hair cells that endogenously express STRC, including cochlear and vestibular hair cells. The STRC promoters described herein may be operably linked to a polynucleotide, such as a transgene, encoding a heterologous expression product and used for the treatment of subjects having or at risk of developing hearing loss or vestibular dysfunction.

### BACKGROUND

Hearing loss is a major public health issue that is estimated to affect nearly 15% of school-age children and one out of three people by age sixty-five. The most common type of hearing loss is sensorineural hearing loss, a type of hearing loss caused by defects in the cells of the inner ear, such as cochlear hair cells, or the neural pathways that project from the inner ear to the brain. Sensorineural hearing loss is often acquired, and has a variety of causes, including acoustic trauma, disease or infection, head trauma, ototoxic drugs, and aging. There are also genetic causes of sensorineural hearing loss, such as mutations in genes involved in the development and function of the inner ear. Mutations in over 90 such genes have been identified, including mutations inherited in an autosomal recessive, autosomal dominant, and X-linked pattern. One such form of autosomal recessive sensorineural hearing loss is associated with mutation of the STRC gene. Stereocilin is a large protein encoded by the STRC gene on chromosome 15q15, which contains 29 exons spanning approximately 19 kb of the genome. The STRC gene is tandemly duplicated, where the second copy contains a premature stop codon in exon 20, thereby producing an STRC pseudogene. Previous studies have identified mutations in STRC in families with autosomal recessive non-syndromic sensorineural hearing loss (Verpy et al., Nat. Genet. 29:345-9 (2001)). Stereocilin protein expression is limited to stereocilia in hair bundles of hair cells and the stereocilin protein is thought to form horizontal top connectors and tectorial membrane-attachment crowns, which are required for the normal functioning of the auditory apparatus (Avan et al., PNAS 116:25948-57 (2019); Verpy et al., J. Comp. Neurol. 519:194-210 (2011)). Mice lacking stereocilin have been shown to exhibit abnormal hair cell bundles with defective cohesion and impaired hearing (Verpy et al., Nature 456:255-8 (2008)).

Factors that disrupt the development, survival, or integrity of cochlear hair cells, such as genetic mutations, disease or infection, ototoxic drugs, head trauma, and aging, may similarly affect vestibular hair cells and are, therefore, also implicated in vestibular dysfunction, including vertigo, dizziness, and imbalance. Indeed, patients carrying mutations that disrupt hair cell development or function can present with both hearing loss and vestibular dysfunction, or either disorder alone. Approximately 35% of US adults aged 40 years and older exhibit balance disorders and this proportion dramatically increases with age, leading to disruption of daily activities, decline in mood and cognition, and an increased prevalence of falls among the elderly.

In recent years, efforts to treat hearing loss and vestibular dysfunction have increasingly focused on gene therapy as a possible solution; however, there remain few approaches to specifically target hair cells in the cochlea or vestibular system, which are frequently implicated in hearing loss and vestibular dysfunction, respectively. There is a need for new therapeutics to target hair cells for the treatment of sensorineural hearing loss and vestibular dysfunction.

### SUMMARY OF THE INVENTION

The invention provides compositions and methods for promoting the expression of a gene of interest, such as a gene that promotes or improves hair cell function, regeneration, or survival, in specific cell types. The compositions and methods described herein relate to polynucleotides that can induce expression of a transgene in cochlear hair cells and vestibular hair cells of the inner ear. The polynucleotides described herein may be operably linked, e.g., to a polynucleotide encoding a desired expression product such as a protein or an inhibitory RNA, and may be administered to a subject, such as a human subject, to treat or prevent hearing loss (e.g., sensorineural hearing loss) or vestibular dysfunction (e.g., vertigo, dizziness, imbalance, bilateral vestibulopathy, oscillopsia, or a balance disorder). The invention also provides two-vector systems including a first nucleic acid vector containing a polynucleotide described herein operably linked to a polynucleotide encoding an N-terminal portion of a stereocilin protein and a second nucleic acid vector containing a polynucleotide encoding a C-terminal portion of the stereocilin protein, which can be used to treat a subject having or at risk of developing hearing loss or vestibular dysfunction associated with a mutation in a stereocilin gene (STRC).

In a first aspect, the invention provides a polynucleotide including a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof that includes nucleotides 252-537 or 35-530 of SEQ ID NO: 2, operably linked to a polynucleotide encoding a heterologous expression product.

In another aspect, the invention provides a polynucleotide including a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof that includes nucleotides 280-560 of SEQ ID NO: 48 operably linked to a polynucleotide encoding a heterologous expression product.

In another aspect, the invention provides a nucleic acid vector containing the polynucleotide of any of the foregoing aspects.

In another aspect, the invention provides a nucleic acid vector containing an STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof including nucleotides 252-537 or 35-530 of SEQ ID NO: 2.

In another aspect, the invention provides a nucleic acid vector containing a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof that includes nucleotides 280-560 of SEQ ID NO: 48.

In some embodiments of any of the foregoing aspects, the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.

In some embodiments of any of the foregoing aspects, the STRC promoter consists of SEQ ID NO: 1.

In some embodiments of any of the foregoing aspects, the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof that includes nucleotides 252-537 or 35-530 of SEQ ID NO: 2.

In some embodiments of any of the foregoing aspects, the functional portion of SEQ ID NO: 2 includes or consists of nucleotides 252-537 of SEQ ID NO: 2. In some embodiments of any of the foregoing aspects, the functional portion of SEQ ID NO: 2 includes or consists of nucleotides 120-537 of SEQ ID NO: 2. In some embodiments of any of the foregoing aspects, the functional portion of SEQ ID NO: 2 includes or consists of nucleotides 35-530 of SEQ ID NO: 2.

In some embodiments of any of the foregoing aspects, the STRC promoter consists of SEQ ID NO: 2.

In some embodiments of any of the foregoing aspects, the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48.

In some embodiments of any of the foregoing aspects, the STRC promoter has the sequence of SEQ ID NO: 48.

In some embodiments of any of the foregoing aspects, the functional portion of SEQ ID NO: 48 includes or consists of nucleotides 280-560 of SEQ ID NO: 48. In some embodiments of any of the foregoing aspects, the functional portion of SEQ ID NO: 48 includes or consists of nucleotides 280-564 of SEQ ID NO: 48. In some embodiments of any of the foregoing aspects, the functional portion of SEQ ID NO: 48 includes or consists of nucleotides 124-560 of SEQ ID NO: 48. In some embodiments of any of the foregoing aspects, the functional portion of SEQ ID NO: 48 includes or consists of nucleotides 124-564 of SEQ ID NO: 48. In some embodiments of any of the foregoing aspects, the functional portion of SEQ ID NO: 48 includes or consists of nucleotides 1-560 of SEQ ID NO: 48.

In some embodiments of any of the foregoing aspects, the STRC promoter is operably linked to a polynucleotide encoding a heterologous expression product.

In some embodiments of any of the foregoing aspects, the heterologous expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA. In some embodiments, the protein is Actin Gamma 1 (ACTG1), Fascin Actin-Bundling Protein 2, Retinal (FSCN2), Radixin (RDX), POU Class 4 Homeobox 3 (POU4F3), TRIO and F-Actin Binding Protein (TRIOBP), Taperin (TPRN), Xin Actin Binding Repeat Containing 2 (XIRP2), Atonal BHLH Transcription Factor 1 (ATOH1), Growth Factor Independent 1 Transcriptional Repressor (GFI1), Cholinergic Receptor Nicotinic Alpha 9 Subunit (CHRNA9), Cholinergic Receptor Nicotinic Alpha 10 Subunit (CHRNA10), Calcium and Integrin Binding Family Member 3 (CIB3), Cadherin 23 (CDH23), Protocadherin 15 (PCDH15), Kinocilin (KNCN), Pejvakin (DFNB59), MKRN2 Opposite Strand (MKRN2OS), LIM Homeobox Protein 3 (LHX3), Transmembrane Channel Like 1 (TMC1), Myosin 15 (MYO15), Myosin 7A (MYO7A), Myosin 6 (MYO6), Myosin IIIA (MYO3A), Myosin IIIB (MYO3B), Glutaredoxin Domain Containing Cysteine-Rich Protein 1 (GRXCR1), Protein Tyrosine Phosphatase, Receptor Type Q (PTPRQ), Late Cornified Envelope 6A (LCE6A), Lipoxygenase Homology Domain-containing Protein 1 (LOXHD1), ADP-Ribosyltransferase 1 (ART1), ATPase Plasma Membrane Ca2+ Transporting 2 (ATP2B2), Calcium and Integrin Binding Family Member 2 (CIB2), Calcium Voltage-Gated Channel Auxiliary Subunit Alpha2delta 4 (CACNA2D4), Epidermal Growth Factor Receptor Pathway Substrate 8 (EPS8), EPS8 Like 2 (EPS8L2), Espin (ESPN), Espin Like (ESPNL), Peripherin 2 (PRPH2), Solute Carrier Family 8 Member A2 (SLC8A2), Zinc Finger CCHC-Type Containing Protein 12 (ZCCHC12), Leucine Rich Transmembrane and O-methyltransferase Domain Containing (LRTOMT2, LRTOMT1), USH1 Protein Network Component Harmonin (USH1C), Solute Carrier Family 26 Member 5 (SLC26A5), Piezo Type Mechanosensitive Ion Channel Component 2 (PIEZO2), Extracellular Leucine Rich Repeat and Fibronectin Type III Domain Containing 1 (ELFN1), Tetratricopeptide Repeat Protein 24 (TTC24), Dystrotelin (DYTN), Coiled-coil Glutamate Rich Protein 2 (CCER2), Leucine-rich Repeat and Transmembrane Domain-containing protein 2 (LRTM2), Potassium Voltage-Gated Channel Subfamily A Member 10 (KCNA10), Clarin 1 (CLRN1), Clarin 2 (CLRN2), SKI Family Transcriptional Corepressor 1 (SKOR1), Tctexl Domain Containing Protein 1 (TCTEX1 D1), Fc Receptor Like B (FCRLB), Glutaredoxin Domain Containing Cysteine-Rich Protein 2 (GRXCR2), Serpin Family E Member 3 (SERPINE3), Nescient Helix-loop Helix 1 (NHLH1), Heat Shock Protein 70 (HSP70), Heat Shock Protein 90 (HSP90), Activating Transcription Factor 6 (ATF6), Eukaryotic Translation Initiation Factor 2 Alpha Kinase 3 (PERK), Serine/Threonine-Protein Kinase/Endoribonuclease IRE1 (IRE1), Whirlin (WHRN), Oncomodulin (OCM), LIM Homeobox 1 (Isl1), Transmembrane and Tetratricopeptide Repeat Containing 4 (TMTC4), Binding Immunoglobulin Protein (BIP), or Potassium Voltage-Gated Channel Subfamily Q Member 4 (KCNQ4).

In some embodiments of any of the foregoing aspects, a linking polynucleotide is used to link the 3′ end of the STRC promoter and the 5′ start site (ATG) of the polynucleotide encoding the protein. In some embodiments, the linking polynucleotide includes a Kozak sequence or a portion thereof. In some embodiments, the linking polynucleotide includes a multiple cloning site or a portion thereof.

In some embodiments of any of the foregoing aspects, the nucleic acid vector is a viral vector, plasmid, cosmid, or artificial chromosome. In some embodiments, the nucleic acid vector is a viral vector. In some embodiments, the viral vector is an adeno-associated virus (AAV) vector, an adenovirus vector, or a lentivirus vector. In some embodiments, the viral vector is an AAV vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid. In some embodiments, the AAV vector has an AAV1 capsid. In some embodiments, the AAV vector has an AAV9 capsid. In some embodiments, the AAV vector has a 7m8 capsid. In some embodiments, the AAV vector has a PHP.S capsid. In some embodiments, the AAV vector has an Anc80 capsid. In some embodiments, the AAV vector has an Anc80L65 capsid. In some embodiments, the AAV vector has an AAV2 capsid. In some embodiments, the AAV vector has an AAV2quad(Y-F) capsid. In some embodiments, the AAV vector has a PHP.eB capsid. In some embodiments, the AAV vector has an AAV3 capsid. In some embodiments, the AAV vector has an AAV4 capsid. In some embodiments, the AAV vector has an AAV5 capsid. In some embodiments, the AAV vector has an AAV6 capsid. In some embodiments, the AAV vector has an AAV7 capsid. In some embodiments, the AAV vector has an AAV8 capsid. In some embodiments, the AAV vector has a PHP.B capsid.

In another aspect, the invention provides a nucleic acid vector including a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof including nucleotides 252-537 or 35-530 of SEQ ID NO: 2, operably linked to a first polynucleotide encoding an N-terminal portion of a stereocilin protein that does not encode a full-length stereocilin protein. In some embodiments, the nucleic acid vector is a first nucleic acid vector in a two-vector system that further includes a second nucleic acid vector containing a second polynucleotide encoding a C-terminal portion of a stereocilin protein.

In another aspect, the invention provides a nucleic acid vector including a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof that includes nucleotides 280-560 of SEQ ID NO: 48, operably linked to a first polynucleotide encoding an N-terminal portion of a stereocilin protein that does not encode a full-length stereocilin protein. In some embodiments, the nucleic acid vector is a first nucleic acid vector in a two-vector system that further includes a second nucleic acid vector containing a second polynucleotide encoding a C-terminal portion of a stereocilin protein.

In another aspect, the invention provides a two-vector system including: (a) a first nucleic acid vector containing a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof that includes nucleotides 252-537 or 35-530 of SEQ ID NO: 2; operably linked to a first polynucleotide encoding an N-terminal portion of a stereocilin protein; and (b) a second nucleic acid vector containing a second polynucleotide encoding a C-terminal portion of a stereocilin protein.

In another aspect, the invention provides a two-vector system including: (a) a first nucleic acid vector containing a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof that includes nucleotides 280-560 of SEQ ID NO: 48 operably linked to a first polynucleotide encoding an N-terminal portion of a stereocilin protein; and (b) a second nucleic acid vector containing a second polynucleotide encoding a C-terminal portion of a stereocilin protein.

In some embodiments, the first polynucleotide partially overlaps with the second polynucleotide. In some embodiments, the first polynucleotide and the second polynucleotide have a region of overlap having a length of at least 200 bases (b) (e.g., at least 200 b, 300 b, 400 b, 500 b, 600 b, 700 b, 800 b, 900 b, 1.0 kilobase (kb), 1.1 kb, 1.2 kb, 1.3 kb, 1.4 kb, 1.5 kb or more). In some embodiments, when introduced into a mammalian cell, the first and second nucleic acid vectors undergo homologous recombination to form a recombined polynucleotide that encodes a full-length stereocilin protein.

In some embodiments, the first nucleic acid vector includes a splice donor signal sequence positioned 3′ of the first polynucleotide and the second nucleic acid vector includes a splice acceptor signal sequence positioned 5′ of the second polynucleotide. In some embodiments, the first and second polynucleotides do not overlap.

In some embodiments, the first nucleic acid vector includes a splice donor signal sequence positioned 3′ of the first polynucleotide and a first recombinogenic region positioned 3′ of the splice donor signal sequence and the second nucleic acid vector includes a second recombinogenic region, a splice acceptor signal sequence positioned 3′ of the recombinogenic region, and the second polynucleotide positioned 3′ of the splice acceptor signal sequence. In some embodiments, the first and second polynucleotides do not overlap. In some embodiments, the first and second recombinogenic regions are the same. In some embodiments, each of the first recombinogenic region and the second recombinogenic region is an AP gene fragment. In some embodiments, the AP gene fragment includes or consists of the sequence of any one of SEQ ID NOs: 42-47. In some embodiments, the AP gene fragment includes or consists of the sequence of SEQ ID NO: 45. In some embodiments, the first nucleic acid vector further includes a degradation signal sequence positioned 3′ of the recombinogenic region and the second nucleic acid vector further includes a degradation signal sequence positioned between the recombinogenic region and the splice acceptor signal sequence.

In some embodiments, the second nucleic acid vector further includes a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof that includes nucleotides 252-537 or 35-530 of SEQ ID NO: 2; operably linked to the second polynucleotide, in which the STRC promoter is positioned 5′ of the second polynucleotide.

In some embodiments, the second nucleic acid vector further includes a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof that includes nucleotides 280-560 of SEQ ID NO: 48 operably linked to the second polynucleotide, in which the STRC promoter is positioned 5′ of the second polynucleotide.

In some embodiments of any of the foregoing aspects, the STRC promoter in the second nucleic acid vector is the same (i.e., has the same nucleotide sequence) as the STRC promoter in the first nucleic acid vector. In some embodiments of any of the foregoing aspects, the STRC promoter in the second nucleic acid vector has a different nucleotide sequence than the STRC promoter in the first nucleic acid vector.

In some embodiments of any of the foregoing aspects, the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.

In some embodiments of any of the foregoing aspects, the STRC promoter consists of SEQ ID NO: 1.

In some embodiments of any of the foregoing aspects, the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof that includes nucleotides 252-537 or 35-530 of SEQ ID NO: 2.

In some embodiments of any of the foregoing aspects, the functional portion of SEQ ID NO: 2 includes or consists of nucleotides 252-537 of SEQ ID NO: 2. In some embodiments of any of the foregoing aspects, the functional portion of SEQ ID NO: 2 includes or consists of nucleotides 120-537 of SEQ ID NO: 2. In some embodiments of any of the foregoing aspects, the functional portion of SEQ ID NO: 2 includes or consists of nucleotides 35-530 of SEQ ID NO: 2.

In some embodiments of any of the foregoing aspects, the STRC promoter consists of SEQ ID NO: 2.

In some embodiments of any of the foregoing aspects, the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48.

In some embodiments of any of the foregoing aspects, the STRC promoter has the sequence of SEQ ID NO: 48.

In some embodiments of any of the foregoing aspects, the functional portion of SEQ ID NO: 48 includes or consists of nucleotides 280-560 of SEQ ID NO: 48. In some embodiments of any of the foregoing aspects, the functional portion of SEQ ID NO: 48 includes or consists of nucleotides 280-564 of SEQ ID NO: 48. In some embodiments of any of the foregoing aspects, the functional portion of SEQ ID NO: 48 includes or consists of nucleotides 124-560 of SEQ ID NO: 48. In some embodiments of any of the foregoing aspects, the functional portion of SEQ ID NO: 48 includes or consists of nucleotides 124-564 of SEQ ID NO: 48. In some embodiments of any of the foregoing aspects, the functional portion of SEQ ID NO: 48 includes or consists of nucleotides 1-560 of SEQ ID NO: 48.

In some embodiments of any of the foregoing aspects, the first nucleic acid vector further includes a polynucleotide encoding an N-terminal intein (N-intein) positioned 3′ of and in reading frame with the first polynucleotide. In some embodiments of any of the foregoing aspects, the second nucleic acid vector further includes a polynucleotide encoding a C-terminal intein (C-intein) positioned between the STRC promoter and the second polynucleotide and in reading frame with the second polynucleotide. In some embodiments, the N-intein and C-intein are components of a split intein trans-splicing system.

In some embodiments of any of the foregoing aspects, the first and/or second vectors include an intein degradation signal. In some embodiments, the degradation signal is an N-degron and/or a C-degron. In some embodiments, the N-degron and/or the C-degron are independently a CL1, PB29, SMN, CIITA, or ODC degron. In some embodiments, the degradation signal is an E. coli dihydrofolate reductase (ecDHFR) degradation signal. In some embodiments the degradation signal is an FKBP12 degradation domain (Banaszynski et al., Cell 126:995-1004, 2006). In some embodiments the degradation signal is a PEST degradation domain (Rechsteiner and Rogers, Trends Biochem Sci. 21:267-271, 1996). In some embodiments the degradation signal is a UbR tag ubiquitination signal (Chassin et al., Nat Commun. 10:2013, 2019). In some embodiments the degradation signal is a destabilized mutation of human ELRBD (Miyazaki et al., J. Am. Chem. Soc., 134:3942-3945, 2012).

In some embodiments of any of the foregoing aspects, the first and second vectors, when introduced into a mammalian (e.g., human) hair cell (e.g., inner hair cell, outer hair cell, Type I vestibular hair cell, or Type II vestibular hair cell), produce a first and second fusion protein, respectively, in which the first fusion protein includes the N-terminal portion of the stereocilin protein and the N-intein positioned 3′ thereto, and the second fusion protein includes the C-intein and the C-terminal portion of the stereocilin protein positioned 3′ thereto. In some embodiments, the C-terminus of the N-intein of the first fusion protein and the N-terminus of the C-intein of the second fusion protein are capable of forming a peptide bond, thereby producing a polypeptide including, from N-terminus to C-terminus, the N-terminal portion of the stereocilin protein, N-intein, C-intein, and the C-terminal portion of the stereocilin protein, in which the bound N-intein and C-intein are capable of self-excising and ligating the C-terminus of the N-terminal portion of the stereocilin protein and the N-terminus of the C-terminal portion of the stereocilin protein, thereby producing a full-length stereocilin protein.

In some embodiments of any of the foregoing aspects, the split intein trans-splicing system is derived from a DnaEgene of one or more bacteria. In some embodiments, the one or more bacteria are selected from the group consisting of Nostoc punctiforme (Npu), Synechocystis sp. PCC6803 (Ssp), Fischerella sp. PCC9605 (Fsp), Scytonema tolypothrichoides (Sto), Cyanobacteria bacterium SW_9_47_5, Nodularia spumigena (Nsp), Nostoc flagelliforme (Nfl), Crocosphaera watsonii (Cwa) WH8502, Chroococcidiopsis cubana (Ccu) CCALA043, Trichodesmium erythraeum (Ter), Rhodothermus marinus (Rma), Saccharomyces cerevisiae (Sce), Saccharomyces castellii (Sca), Saccharomyces unisporus (Sun), Zygosaccharomyces bisporus (Zbi), Torulaspora pretoriensis (Tpr), Mycobacteria tuberculosis (Mtu), Mycobacterium leprae (Mle), Mycobacterium smegmatis (Msm), Pyrococcus abyssi (Pab), Pyrococcus horikoshii (Pho), Coxiella burnetti (Cbu), Coxiella neoformans (Cne), Coxiella gattii (Cga), Histoplasma capsulatum (Hca), and Porphyra purpurea chloroplast (Ppu). In some embodiments, the split intein trans-splicing system is derived from multiple sequence alignment studies of DnaE for identifying a consensus design (e.g., Cfa) to engineer a split intein with desirable stability and activity.

In some embodiments of any of the foregoing aspects, the N-intein has a sequence of any one of SEQ ID NOs: 7, 9, 12, 14, 16-21, 26, 28, 30, 32, 34, 36, 38, 49, 51, 53, 55, and 57 and the C-intein has a sequence of any one of SEQ ID NOs: 8, 10, 11, 13, 15, 22-25, 27, 29, 31, 33, 35, 37, 39, 50, 52, 54, 56, and 58. In some embodiments, the N-intein has the sequence of SEQ ID NO: 7 and the C-intein has the sequence of SEQ ID NO: 8. In some embodiments, the N-intein has the sequence of SEQ ID NO: 7 and the C-intein has the sequence of SEQ ID NO: 10. In some embodiments, the N-intein has the sequence of SEQ ID NO: 7 and the C-intein has the sequence of SEQ ID NO: 11. In some embodiments, the N-intein has the sequence of SEQ ID NO: 9 and the C-intein has the sequence of SEQ ID NO: 8. In some embodiments, the N-intein has the sequence of SEQ ID NO: 9 and the C-intein has the sequence of SEQ ID NO: 10. In some embodiments, the N-intein has the sequence of SEQ ID NO: 9 and the C-intein has the sequence of SEQ ID NO: 11. In some embodiments, the N-intein has the sequence of SEQ ID NO: 12 and the C-intein has the sequence of SEQ ID NO: 13. In some embodiments, the N-intein has the sequence of SEQ ID NO: 14 and the C-intein has the sequence of SEQ ID NO: 15. In some embodiments, the N-intein has the sequence of SEQ ID NO: 16 and the C-intein has the sequence of SEQ ID NO: 22. In some embodiments, the N-intein has the sequence of SEQ ID NO: 19 and the C-intein has the sequence of SEQ ID NO: 23. In some embodiments, the N-intein has the sequence of SEQ ID NO: 20 and the C-intein has the sequence of SEQ ID NO: 24. In some embodiments, the N-intein has the sequence of SEQ ID NO: 21 and the C-intein has the sequence of SEQ ID NO: 25. In some embodiments, the N-intein has the sequence of SEQ ID NO: 26 and the C-intein has the sequence of SEQ ID NO: 27. In some embodiments, the N-intein has the sequence of SEQ ID NO: 28 and the C-intein has the sequence of SEQ ID NO: 29. In some embodiments, the N-intein has the sequence of SEQ ID NO: 30 and the C-intein has the sequence of SEQ ID NO: 31. In some embodiments, the N-intein has the sequence of SEQ ID NO: 32 and the C-intein has the sequence of SEQ ID NO: 33. In some embodiments, the N-intein has the sequence of SEQ ID NO: 34 and the C-intein has the sequence of SEQ ID NO: 35. In some embodiments, the N-intein has the sequence of SEQ ID NO: 36 and the C-intein has the sequence of SEQ ID NO: 37. In some embodiments, the N-intein has the sequence of SEQ ID NO: 38 and the C-intein has the sequence of SEQ ID NO: 39. In some embodiments, the N-intein has the sequence of any one of SEQ ID NOs: 16-21 and the C-intein has the sequence of any one of SEQ ID NOs: 22-25. In some embodiments, the N-intein has the sequence of SEQ ID NO: 49 and the C-intein has the sequence of SEQ ID NO: 50. In some embodiments, the N-intein has the sequence of SEQ ID NO: 51 and the C-intein has the sequence of SEQ ID NO: 52. In some embodiments, the N-intein has the sequence of SEQ ID NO: 53 and the C-intein has the sequence of SEQ ID NO: 54. In some embodiments, the N-intein has the sequence of SEQ ID NO: 55 and the C-intein has the sequence of SEQ ID NO: 56. In some embodiments, the N-intein has the sequence of SEQ ID NO: 57 and the C-intein has the sequence of SEQ ID NO: 58. In some embodiments, the split intein trans-splicing system includes one or more inteins that perform protein trans-splicing only upon contact with a ligand. In some embodiments, the ligand is selected from the group consisting of 4-hydroxytamoxifen, a peptide, a protein, a polynucleotide, an amino acid, and a nucleotide.

In some embodiments of any of the foregoing aspects, a linking polynucleotide is used to link the 3′ end of the STRC promoter and the 5′ start site (ATG) of the first polynucleotide and/or the polynucleotide encoding a C-intein. In some embodiments, the linking polynucleotide includes a Kozak sequence or a portion thereof. In some embodiments, the linking polynucleotide includes a multiple cloning site or a portion thereof.

In some embodiments of any of the foregoing aspects, the first nucleic acid vector further includes a polynucleotide encoding a signal peptide. In some embodiments, the polynucleotide encoding a signal peptide is placed 5′ of and in frame with the polynucleotide encoding the N-terminal portion of the stereocilin protein. In some embodiments of any of the foregoing aspects, the second nucleic acid vector further includes a polynucleotide encoding a signal peptide. In some embodiments, the polynucleotide encoding a signal peptide is placed 5′ of and in frame with the polynucleotide encoding the C-terminal portion of the stereocilin protein.

In some embodiments of any of the foregoing aspects, neither the first nor the second polynucleotide encodes a full-length stereocilin protein. In some embodiments of any of the foregoing aspects, each of the first and second polynucleotides encodes about half of the stereocilin protein sequence.

In some embodiments of any of the foregoing aspects, the second nucleic acid vector further includes a poly(A) sequence 3′ of the second polynucleotide.

In some embodiments of any of the foregoing aspects, the first and second nucleic acid vectors do not include STRC untranslated regions (UTRs) that are not part of the promoters described herein. In some embodiments of any of the foregoing aspects, the first and second nucleic acid vectors include STRC UTRs. In some embodiments of any of the foregoing aspects, the first nucleic acid vector includes a 5′ STRC UTR 5′ of the first polynucleotide. In some embodiments of any of the foregoing aspects, the second nucleic acid vector includes a 3′ STRC UTR 3′ of the second polynucleotide.

In some embodiments of any of the foregoing aspects, the first and second polynucleotides that encode portions of the stereocilin protein do not include introns (e.g., the first and second polynucleotides are portions of STRC cDNA). In some embodiments of any of the foregoing aspects, the first and second polynucleotides that encode portions of the stereocilin protein include introns.

In some embodiments of any of the foregoing aspects, the two-vector system is capable of directing hair cell-specific expression of a full-length stereocilin protein in a mammalian hair cell. In some embodiments, the mammalian hair cell is a human hair cell. In some embodiments, the mammalian hair cell is a murine hair cell. In some embodiments, the hair cell is a cochlear hair cell. In some embodiments, the cochlear hair cell is an outer hair cell. In some embodiments, the cochlear hair cell is an inner hair cell. In some embodiments, the hair cell is a vestibular hair cell. In some embodiments, the vestibular hair cell is a Type I vestibular hair cell. In some embodiments, the vestibular hair cell is a Type II vestibular hair cell.

In some embodiments of any of the foregoing aspects, the stereocilin protein is a human stereocilin protein having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 3. In some embodiments, the stereocilin protein has the sequence of SEQ ID NO: 3. In some embodiments, the human stereocilin protein is encoded by a polynucleotide having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 5. In some embodiments, the polynucleotide that has at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 5 encodes the stereocilin protein of SEQ ID NO: 3. In some embodiments, the human stereocilin protein is encoded by a polynucleotide having the sequence of SEQ ID NO: 5.

In some embodiments, the stereocilin protein is a murine stereocilin protein having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 4. In some embodiments, the stereocilin protein has the sequence of SEQ ID NO: 4. In some embodiments, the murine stereocilin protein is encoded by a polynucleotide having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 6. In some embodiments, the polynucleotide that has at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 6 encodes the stereocilin protein of SEQ ID NO: 4. In some embodiments, the murine stereocilin protein is encoded by a polynucleotide having the sequence of SEQ ID NO: 6.

In some embodiments of any of the foregoing aspects, the first and second vectors are viral vectors, plasmids, cosmids, or artificial chromosomes. In some embodiments, the first and second vectors are viral vectors. In some embodiments, the viral vectors are AAV vectors, adenovirus vectors, or lentivirus vectors. In some embodiments, the first and second vectors are AAV vectors. In some embodiments, each of the first and second AAV vectors has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid. In some embodiments, each of the first and second AAV vectors has an AAV1 capsid. In some embodiments, each of the first and second AAV vectors has an AAV9 capsid. In some embodiments, each of the first and second AAV vectors has a 7m8 capsid. In some embodiments, each of the first and second AAV vectors has a PHP.S capsid. In some embodiments, each of the first and second AAV vectors has an Anc80 capsid. In some embodiments, each of the first and second AAV vectors has an Anc80L65 capsid. In some embodiments, each of the first and second AAV vectors has an AAV2 capsid. In some embodiments, each of the first and second AAV vectors has an AAV2quad(Y-F) capsid. In some embodiments, each of the first and second AAV vectors has a PHP.eB capsid. In some embodiments, each of the first and second AAV vectors has an AAV3 capsid. In some embodiments, each of the first and second AAV vectors has an AAV4 capsid. In some embodiments, each of the first and second AAV vectors has an AAV5 capsid. In some embodiments, each of the first and second AAV vectors has an AAV6 capsid. In some embodiments, each of the first and second AAV vectors has an AAV7 capsid. In some embodiments, each of the first and second AAV vectors has an AAV8 capsid. In some embodiments, each of the first and second AAV vectors has a PHP.B capsid.

In another aspect, the invention provides a composition containing the nucleic acid vector or two-vector system of any of the foregoing aspects or embodiments. In some embodiments, the composition further includes a pharmaceutically acceptable carrier, diluent, or excipient.

In another aspect, the invention provides a cell containing the polynucleotide, nucleic acid vector, or two-vector system of any of the foregoing aspects or embodiments. In some embodiments, the cell is a hair cell. In some embodiments, the hair cell is a mammalian hair cell. In some embodiments, the mammalian hair cell is a human hair cell. In some embodiments, the hair cell is a cochlear hair cell. In some embodiments, the cochlear hair cell is an outer hair cell. In some embodiments, the cochlear hair cell is an inner hair cell. In some embodiments, the hair cell is a vestibular hair cell. In some embodiments, the vestibular hair cell is a type II vestibular hair cell. In some embodiments, the vestibular hair cell is a type I vestibular hair cell.

In another aspect, the invention provides a method of expressing a heterologous expression product in a hair cell by contacting the hair cell with the nucleic acid vector or composition of any of the foregoing aspects or embodiments. In some embodiments, the contacting is in vivo (e.g., in a subject). In some embodiments, the expression product is specifically expressed in hair cells.

In another aspect, the invention provides a method of expressing a stereocilin protein in a hair cell by contacting the hair cell with the two-vector system or composition of any of the foregoing aspects or embodiments. In some embodiments, the contacting is in vivo (e.g., in a subject). In some embodiments, the stereocilin protein is specifically expressed in hair cells.

In another aspect, the invention provides a method of treating a subject having or at risk of developing hearing loss (e.g., sensorineural hearing loss, nonsyndromic hearing loss, auditory neuropathy, or deafness) by administering to an inner ear of the subject an effective amount of the nucleic acid vector, two-vector system, or composition of any of the foregoing aspects or embodiments.

In another aspect, the invention provides a method of treating a subject having or at risk of developing tinnitus by administering to an inner ear of the subject an effective amount of the nucleic acid vector or composition of any of the foregoing aspects or embodiments.

In another aspect, the invention provides a method of treating a subject having or at risk of developing vestibular dysfunction by administering to an inner ear of the subject an effective amount of the nucleic acid vector, two-vector system, or composition of any of the foregoing aspects or embodiments.

In another aspect, the invention provides a method of treating a subject having or at risk of developing bilateral vestibulopathy by administering to an inner ear of the subject an effective amount of the nucleic acid vector, two-vector system, or composition of any of the foregoing aspects or embodiments. In some embodiments, the bilateral vestibulopathy is ototoxic drug-induced bilateral vestibulopathy.

In another aspect, the invention provides a method of treating a subject having or at risk of developing oscillopsia by administering to an inner ear of the subject an effective amount of the nucleic acid vector, two-vector system, or composition of any of the foregoing aspects or embodiments. In some embodiments, the oscillopsia is ototoxic drug-induced oscillopsia.

In another aspect, the invention provides a method of treating a subject having or at risk of developing a balance disorder by administering to an inner ear of the subject an effective amount of the nucleic acid vector, two-vector system, or composition of any of the foregoing aspects or embodiments.

In another aspect, the invention provides a method of inducing or increasing hair cell regeneration in a subject in need thereof by administering to an inner ear of the subject an effective amount of the nucleic acid vector or composition of any of the foregoing aspects or embodiments.

In another aspect, the invention provides a method of increasing hair cell maintenance in a subject in need thereof by administering to an inner ear of the subject an effective amount of the nucleic acid vector, two-vector system, or composition of any of the foregoing aspects or embodiments.

In another aspect, the invention provides a method of increasing hair cell survival in a subject in need thereof by administering to an inner ear of the subject an effective amount of the nucleic acid vector, two-vector system, or composition of any of the foregoing aspects or embodiments.

In another aspect, the invention provides a method of inducing or increasing hair cell maturation in a subject in need thereof by administering to an inner ear of the subject an effective amount of the nucleic acid vector or composition of any of the foregoing aspects or embodiments.

In another aspect, the invention provides a method of preventing or reducing ototoxic drug-induced hair cell damage or death in a subject in need thereof by administering to an inner ear of the subject an effective amount of the nucleic acid vector, two-vector system, or composition of any of the foregoing aspects or embodiments.

In another aspect, the invention provides a method of preventing or reducing hair cell damage or death in a subject in need thereof by administering to an inner ear of the subject an effective amount of the nucleic acid vector, two-vector system, or composition of any of the foregoing aspects or embodiments.

In another aspect, the invention provides a method of improving hair cell function in a subject in need thereof by administering to an inner ear of the subject an effective amount of the nucleic acid vector, two-vector system, or composition of any of the foregoing aspects or embodiments.

In another aspect, the invention provides a method of increasing or improving hair bundle attachment (e.g., OHC hair bundle attachment) to the tectorial membrane in a subject in need thereof, including administering to an inner ear of the subject an effective amount of the nucleic acid vector, two-vector system, or composition of any of the foregoing aspects or embodiments.

In another aspect, the invention provides a method of increasing STRC expression (e.g., wild-type STRC expression, e.g., to produce wild-type stereocilin protein) in a subject in need thereof (e.g., in a hair cell in the subject) by administering to an inner ear of the subject a therapeutically effective amount of the two-vector system or composition of any of the foregoing aspects or embodiments.

In some embodiments of any of the foregoing aspects, the hair cell is a mammalian hair cell. In some embodiments, the mammalian hair cell is a human hair cell. In some embodiments of any of the foregoing aspects, the hair cell is a hair cell that endogenously expresses STRC. In some embodiments of any of the foregoing aspects, the hair cell is a cochlear hair cell. In some embodiments, the cochlear hair cell is an outer hair cell. In some embodiments, the cochlear hair cell is an inner hair cell. In some embodiments of any of the foregoing aspects, the hair cell is a vestibular hair cell. In some embodiments, the vestibular hair cell is a type II vestibular hair cell. In some embodiments, the vestibular hair cell is a type I vestibular hair cell.

In some embodiments of any of the foregoing aspects, the subject has or is at risk of developing hearing loss (e.g., sensorineural hearing loss, such as nonsyndromic hearing loss, auditory neuropathy, or deafness). In some embodiments of any of the foregoing aspects, the hearing loss is genetic hearing loss. In some embodiments, the genetic hearing loss is autosomal dominant hearing loss, autosomal recessive hearing loss, or X-linked hearing loss. In some embodiments of any of the foregoing aspects, the hearing loss is acquired hearing loss. In some embodiments, the acquired hearing loss is noise-induced hearing loss, age-related hearing loss, disease or infection-related hearing loss, head trauma-related hearing loss, or ototoxic drug-induced hearing loss.

In some embodiments of any of the foregoing aspects, the subject has or is at risk of developing vestibular dysfunction. In some embodiments of any of the foregoing aspects, the vestibular dysfunction is vertigo, dizziness, imbalance, bilateral vestibulopathy, oscillopsia, or a balance disorder. In some embodiments of any of the foregoing aspects, the vestibular dysfunction is age-related vestibular dysfunction, head trauma-related vestibular dysfunction, disease or infection-related vestibular dysfunction, or ototoxic drug-induced vestibular dysfunction. In some embodiments of any of the foregoing aspects, the vestibular dysfunction is associated with a genetic mutation. In some embodiments of any of the foregoing aspects, the vestibular dysfunction is idiopathic vestibular dysfunction.

In some embodiments of any of the foregoing aspects, the ototoxic drug is an aminoglycoside (e.g., gentamycin, neomycin, streptomycin, tobramycin, kanamycin, vancomycin, or amikacin), an antineoplastic drug (e.g., a platinum-containing chemotherapeutic agent, such as cisplatin, carboplatin, and oxaliplatin), ethacrynic acid, furosemide, a salicylate (e.g., aspirin, particularly at high doses), or quinine.

In some embodiments of any of the foregoing aspects, the hearing loss, vestibular dysfunction, or tinnitus is associated with loss of hair cells, damage to hair cells, or dysfunction of hair cells (e.g., cochlear and/or vestibular hair cells). In some embodiments of any of the foregoing aspects, the hearing loss or vestibular dysfunction is associated with abnormal hair cell stereocilia bundle deflection or impaired connectivity between the hair bundles (e.g., OHC hair bundles) and the tectorial membrane.

In some embodiments of any of the foregoing aspects, the subject has a mutation in STRC. In some embodiments of any of the foregoing aspects, the subject has been identified as having a mutation in STRC. In some embodiments of any of the foregoing aspects, the method further includes identifying the subject as having a mutation in STRC prior to administering the two-vector system or pharmaceutical composition. In some embodiments of any of the foregoing aspects, the subject has deafness, autosomal recessive 16 (DFNB116). In some embodiments of any of the foregoing aspects, the subject has been identified as having DFNB16.

In some embodiments of any of the foregoing aspects, the method further includes evaluating the hearing of the subject prior to administering the nucleic acid vector, two-vector system, or composition (e.g., evaluating hearing using standard tests, such as audiometry, auditory brainstem response (ABR), electrocochleography (ECOG), or otoacoustic emissions).

In some embodiments of any of the foregoing aspects, the method further includes evaluating the hearing of the subject after administering the nucleic acid vector, two-vector system, or composition (e.g., evaluating hearing using standard tests, such as audiometry, ABR, ECOG, or otoacoustic emissions).

In some embodiments of any of the foregoing aspects, the method further includes evaluating the vestibular function of the subject prior to administering the nucleic acid vector, two-vector system, or composition (e.g., evaluating vestibular function using standard tests, such as an electronystagmogram (ENG) or videonystagmogram (VNG), a test of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), or a specialized clinical balance test, such as those described in Mancini and Horak, Eur J Phys Rehabil Med, 46:239 (2010)).

In some embodiments of any of the foregoing aspects, the method further includes evaluating the vestibular function of the subject after administering the nucleic acid vector, two-vector system, or composition (e.g., evaluating vestibular function using standard tests, such as an ENG, VNG, test of the VOR, posturography, rotary-chair testing, ECOG, VEMP, or a specialized clinical balance test).

In some embodiments of any of the foregoing aspects, the nucleic acid vector, two-vector system, or composition is locally administered. In some embodiments, the nucleic acid vector, two-vector system, or composition is administered to the inner ear. In some embodiments, the nucleic acid vector, two-vector system, or composition is administered to the middle ear. In some embodiments, the nucleic acid vector, two-vector system, or composition is administered to a semicircular canal. In some embodiments, the nucleic acid vector, two-vector system, or composition is administered transtympanically or intratympanically. In some embodiments, the nucleic acid vector, two-vector system, or composition is administered into the perilymph. In some embodiments, the nucleic acid vector, two-vector system, or composition is administered into the endolymph. In some embodiments, the nucleic acid vector, two-vector system, or composition is administered to or through the oval window. In some embodiments, the nucleic acid vector, two-vector system, or composition is administered to or through the round window.

In some embodiments of any of the foregoing aspects, the vectors in the two-vector system are administered concurrently. In some embodiments of any of the foregoing aspects, the vectors in the two-vector system are administered sequentially.

In some embodiments of any of the foregoing aspects, the nucleic acid vector, two-vector system, or composition is administered in an amount sufficient to prevent or reduce vestibular dysfunction, delay the development of vestibular dysfunction, slow the progression of vestibular dysfunction, improve vestibular function, prevent or reduce hearing loss, prevent or reduce tinnitus, delay the development of hearing loss, slow the progression of hearing loss, improve hearing, improve speech discrimination, improve hair cell function, increase STRC expression in a hair cell, increase cochlear and/or vestibular hair cell numbers, increase cochlear and/or vestibular hair cell maturation, increase cochlear and/or vestibular hair cell regeneration, improve cochlear and/or vestibular hair cell function, prevent or reduce cochlear and/or vestibular hair cell damage, prevent or reduce cochlear and/or vestibular hair cell death, improve hair bundle attachment (e.g., OHC hair bundle attachment) to the tectorial membrane, or promote or increase cochlear and/or vestibular hair cell survival.

In some embodiments of any of the foregoing aspects, the subject is a human subject.

In another aspect, the invention provides a kit containing the polynucleotide, nucleic acid vector, two-vector system, or composition of any of the foregoing aspects or embodiments.

### Definitions

As used herein, the term “about” refers to a value that is within 10% above or below the value being described.

As used herein, “administration” refers to providing or giving a subject a therapeutic agent (e.g., a nucleic acid vector containing an STRC promoter operably linked to a transgene), by any effective route. Exemplary routes of administration are described herein below.

As used herein, the phrase “administering to the inner ear” refers to providing or giving a therapeutic agent described herein to a subject by any route that allows for transduction of inner ear cells. Exemplary routes of administration to the inner ear include administration into the perilymph or endolymph, such as to or through the oval window, round window, or semicircular canal (e.g., horizontal canal), or by transtympanic or intratympanic injection, e.g., administration to a hair cell.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in table 1, below.

TABLE 1
Representative physicochemical properties of naturally occurring amino acids

| | | | | Electrostatic | |
|---|---|---|---|---|---|
| | | | Side- | character at | |
| | 3 Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms | |
| | | | | in equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L, and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the term “degradation signal sequence” refers to a sequence (e.g., a nucleotide sequence that can be translated into an amino acid sequence) that mediates the degradation of a polypeptide in which it is contained. Degradation signal sequences can be included in the nucleic acid vectors of the disclosure to reduce or prevent the expression of portions of stereocilin proteins that have not undergone recombination and/or splicing.

The terms “derived” and “derivative” as used herein refer to a nucleic acid, peptide, or protein or a variant or analog thereof comprising one or more mutations and/or chemical modifications as compared to a corresponding full-length wild-type nucleic acid, peptide, or protein. Non-limiting examples of chemical modifications involving nucleic acids include, for example, modifications to the base moiety, sugar moiety, phosphate moiety, phosphate-sugar backbone, or a combination thereof.

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating sensorineural hearing loss or vestibular dysfunction, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” refers to a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human hair cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. The term “expression product” refers to a protein or RNA molecule produced by any of these events.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human hair cell). Exogenous materials include those that are provided from an external source to an organism or to cultured matter extracted there from.

As used herein, the term “exon” refers to a region within the coding region of a gene, the nucleotide sequence of which determines the amino acid sequence of the corresponding protein. The term exon also refers to the corresponding region of the RNA transcribed from a gene. Exons are transcribed into pre-mRNA and may be included in the mature mRNA depending on the alternative splicing of the gene. Exons that are included in the mature mRNA following processing are translated into protein, wherein the sequence of the exon determines the amino acid composition of the protein.

As used herein, the term “functional portion,” when referring to a promoter sequence described herein (e.g., a STRC promoter sequence), refers to a nucleotide sequence that is shorter than a promoter sequence provided in Table 2 (e.g., SEQ ID NO: 2 or SEQ ID NO: 48) and is capable of recruiting RNA polymerase and driving transcription of a gene to which it is operably linked. For example, in the context of the present disclosure, a functional portion of the murine STRC promoter of SEQ ID NO: 2 (537 bases (b)) may have the sequence of or include nucleotides 252-537 of SEQ ID NO: 2. Other functional portions of the STRC promoter of SEQ ID NO: 2 may have the sequence of or include nucleotides 120-537 or nucleotides 35-530 of SEQ ID NO: 2. In another example, a functional portion of the human STRC promoter of SEQ ID NO: 48 (564 bases (b)) may have the sequence of or include nucleotides 280-560 of SEQ ID NO: 48. Other functional portions of the STRC promoter of SEQ ID NO: 48 may have the sequence of or include nucleotides 280-564, nucleotides 124-560, nucleotides 124-564, nucleotides 61-560 (set forth in SEQ ID NO: 1), or nucleotides 1-560 of SEQ ID NO: 48.

As used herein, the term “heterologous” refers to a combination of elements that is not naturally occurring. For example, a heterologous transgene refers to a transgene that is not naturally expressed by the promoter to which it is operably linked.

As used herein, the term “hair cell” refers to a specialized sensory cell of the inner ear that transduces auditory (i.e., a cochlear hair cell) or vestibular (i.e., a vestibular hair cell) information. Hair cells are characterized by bundles of stereocilia that emanate from the apical surface of the cell. Examples of auditory (i.e., cochlear) hair cells include inner hair cells (IHCs) and outer hair cells (OHCs). Examples of vestibular hair cells include Type I vestibular hair cells and Type II vestibular hair cells.

As used herein, the term “hair cell-specific expression” refers to production of an RNA transcript or polypeptide primarily within hair cells (e.g., cochlear hair cells and/or vestibular hair cells) as compared to other cell types of the inner ear (e.g., spiral ganglion neurons, glia, or other inner ear cell types). Hair cell-specific expression of a transgene can be confirmed by comparing transgene expression (e.g., RNA or protein expression) between various cell types of the inner ear (e.g., hair cells vs. non-hair cells) using any standard technique (e.g., quantitative RT PCR, immunohistochemistry, western blot analysis, or measurement of the fluorescence of a reporter (e.g., GFP) operably linked to a promoter). A hair cell-specific promoter induces expression (e.g., RNA or protein expression) of a transgene to which it is operably linked that is at least 50% greater (e.g., 50%, 75%, 100%, 125%, 150%, 175%, 200% greater or more) in hair cells compared to at least 3 (e.g., 3, 4, 5, 6, 7, 8, 9, 10, or more) of the following inner ear cell types: Border cells, inner phalangeal cells, inner pillar cells, outer pillar cells, first row Deiter cells, second row Deiter cells, third row Deiter cells, Hensen's cells, Claudius cells, inner sulcus cells, outer sulcus cells, spiral prominence cells, root cells, interdental cells, basal cells of the stria vascularis, intermediate cells of the stria vascularis, marginal cells of the stria vascularis, spiral ganglion neurons, Schwann cells. A hair cell specific promoter does not have to induce expression in all hair cells but induces expression in at least one of one of the following hair cell types: inner hair cells, outer hair cells, type I vestibular hair cells, or type II vestibular hair cells. The STRC promoters described herein (e.g., SEQ ID NO: 1, SEQ ID NO: 2, and SEQ ID NO: 48, and portions thereof) are hair cell-specific promoters.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “intein,” also referred to as “protein intron,” refers to a portion of a protein that is typically 100-900 amino acid residues long and that is capable of self-excision and ligation of the flanking protein fragments (“exteins”) with a peptide bond. Inteins are produced during protein splicing. The term “intein” subsumes four different classes of inteins, including maxi-intein, mini-intein, trans-splicing intein, and alanine intein. Maxi-inteins refer to N- and C-terminal splicing regions of a protein containing an endonuclease domain. Endonuclease domains, also known as “homing endonuclease genes” or “HEGs” refer to a class of endonucleases encoded as stand-alone genes within introns, as protein fusions with other proteins, or as self-splicing inteins. HEGs generally hydrolyze very few and often targeted DNA regions. Once a HEG hydrolyzes a piece of DNA, the gene encoding the HEG typically incorporates itself into the cleavage site, thereby increasing its allele frequency. Mini-inteins refer to N- and C-terminal splicing domains lacking the endonuclease domain. Trans-splicing inteins refer to inteins that are split into two or more domains which are further split into N-termini and C-termini. Alanine inteins refer to inteins having a splicing junction of an alanine instead of a cysteine or serine. An intein of a precursor protein may come in two genes; in such cases, the intein is designated a split “intein.”

As used herein, the term “intron” refers to a region within the coding region of a gene, the nucleotide sequence of which is not translated into the amino acid sequence of the corresponding protein. The term intron also refers to the corresponding region of the RNA transcribed from a gene. Introns are transcribed into pre-mRNA, but are removed during processing, and are not included in the mature mRNA.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker polynucleotide (e.g., an intervening non-coding polynucleotide) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

As used herein, the term “recombinogenic region” refers to a region of homology that mediates recombination between two different sequences.

As used herein, the term “regulatory sequence” includes promoters, enhancers, and other expression control elements (e.g., polyadenylation signals) that control the transcription or translation of polynucleotides. Such regulatory sequences are described, for example, in Goeddel, Gene Expression Technology: Methods in Enzymology 185 (Academic Press, San Diego, CA, 1990); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “stereocilin” and “STRC” (also known as DFNB16) refer to a protein encoded by the STRC gene and to the gene encoding this protein, respectively. In humans, the STRC gene is tandemly duplicated, where the second copy contains a premature stop codon in exon 20, thereby producing a STRC pseudogene. In the context of the present disclosure, STRC does not refer to the STRC pseudogene. Previous studies have identified mutations in the full-length copy of STRC in human patients with autosomal recessive non-syndromic sensorineural hearing loss (Verpy et al., Nat. Genet. 29:345-9 (2001)). Stereocilin protein expression is limited to stereocilia in hair bundles of hair cells. Stereocilin is thought to form horizontal top connectors and tectorial membrane-attachment crowns, which are required for the normal functioning of the auditory apparatus (Avan et al., PNAS 116:25948-57 (2019); Verpy et al., J. Comp. Neurol. 519:194-210 (2011)). Mice lacking stereocilin have been shown to exhibit abnormal hair cell bundles with defective cohesion and impaired hearing (Verpy et al., Nature 456:255-8 (2008)). The present disclosure provides polynucleotides encoding the full-length stereocilin protein, which, when incorporated into the vector systems described herein, may be used as a therapeutic agent for the treatment of hearing loss (e.g., sensorineural hearing loss) or vestibular dysfunction (e.g., vertigo, dizziness, imbalance, bilateral vestibulopathy, oscillopsia, or a balance disorder) in subjects in need thereof. The terms “stereocilin” and “STRC” also refer to variants of wild-type stereocilin protein and nucleic acids encoding the same, respectively, such as variant proteins having at least 85% sequence identity (e.g., 86%, 87%, 88%, 89%, 90%, 91%, 92%, 93%, 94%, 95%, 96%, 97%, 98%, 99%, or 99.9% identity, or more sequence identity) to the amino acid sequence of a wild-type stereocilin protein (e.g., SEQ ID NO: 3 or SEQ ID NO: 4) or polynucleotides having at least 85% sequence identity (e.g., 86%, 87%, 88%, 89%, 90%, 91%, 92%, 93%, 94%, 95%, 96%, 97%, 98%, 99%, or 99.9% identity, or more sequence identity) to the nucleic acid sequence of a wild-type STRC gene (e.g., SEQ ID NO: 5 or SEQ ID NO: 6), provided that the stereocilin analog encoded retains the therapeutic function of wild-type (WT) stereocilin.

As used herein, the term “STRC promoter” refers to promoter sequences having at least 85% sequence identity (e.g., 85%, 86%, 87%, 88%, 89%, 90%, 91%, 92%, 93%, 94%, 95%, 96%, 97%, 98%, 99%, or 99.9% identity, or more sequence identity) to SEQ ID NO: 1, SEQ ID NO: 2, SEQ ID NO: 48, a functional portion of SEQ ID NO: 2, such as, e.g., a portion containing nucleotides 252-537 or 35-530 of SEQ ID NO: 2, or a functional portion of SEQ ID NO: 48, such as a portion containing nucleotides 280-560 of SEQ ID NO: 48.

As used herein, the term “transcription regulatory element” refers to a polynucleotide that controls, at least in part, the transcription of a gene of interest. Transcription regulatory elements may include promoters, enhancers, and other polynucleotides (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “subject” and “patient” refer to an animal (e.g., a mammal, such as a human). A subject to be treated according to the methods described herein may be one who has been diagnosed with hearing loss (e.g., sensorineural hearing loss) or vestibular dysfunction (e.g., vertigo, dizziness, imbalance, bilateral vestibulopathy, oscillopsia, or a balance disorder) or one at risk of developing one or both of these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” refers to a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, MA, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “vestibular hair cell” refers to a type of specialized cell in the inner ear that is involved in sensing movement and contributes to the sense of balance and spatial orientation. There are two types of vestibular hair cells: Type I and Type II hair cells. Type I hair cells have calyx nerve endings, fast voltage responses, and encode dynamic movements. Type II hair cells have bouton nerve endings, slower voltage responses, and encode slow or static movements. Vestibular hair cells are located in the semicircular canal end organs and otolith organs of the inner ear. Damage to vestibular hair cells and genetic mutations that disrupt vestibular hair cell function are implicated in vestibular dysfunction such as vertigo, dizziness, imbalance, bilateral vestibular hypofunction, oscillopsia, and balance disorders.

As used herein, the term “wild-type” refers to a genotype with the highest frequency for a particular gene in a given organism.

### BRIEF DESCRIPTION OF THE DRAWINGS

FIG. 1 is a map of plasmid P1208.

FIG. 2 is a map of plasmid P1209.

FIG. 3 is a series of micrographs from the organ of Corti of a neonatal mouse administered an AAV vector expressing GFP under control of the STRC promoter of nucleotides 35-530 of SEQ ID NO: 2derived from plasmid P1209. Panels A and B show composite serial sections from different planes of the organ of Corti oriented left to right from base to apex. Panels A and A′ show staining for Myo7a. Panel A′ is a higher magnification of the area shown in a rectangle in panel A. Panels B and B′ show staining for GFP. Panel B′ is a higher magnification of the area shown in a rectangle in panel B. The scale bars represent 100 μm in panels A and B; 50 μm in panels A′ and B′.

FIG. 4 is a series of micrographs from the organ of Corti of a neonatal mouse administered an AAV vector expressing GFP under control of the STRC promoter of SEQ ID NO: 1 derived from plasmid P1208. Panels A and B show composite serial sections from different planes of the organ of Corti oriented left to right from base to apex. Panels A, A′ and A″ show staining for Myo7a. Panel A′ is a higher magnification of the area shown in a solid rectangle in panel A. Panel A″ is a higher magnification of the area shown in a dashed square in panel A. Panels B, B′ and B″ show staining for GFP. Panel B′ is a higher magnification of the area shown in a rectangle in panel B. Panel B″ is a higher magnification of the area shown in a dashed square in panel B. The scale bars represent 100 am in panels A and B; 50 μm in panels A′, A″, B′ and B″.

FIG. 5 is a series of micrographs of a composite section of the vestibule of a neonatal mouse administered the same AAV vector expressing GFP under control of the STRC promoter of nucleotides 35-530 of SEQ ID NO: 2 derived from plasmid P1209. Panels A, A′ and A″ show staining for Pou4f3. Panels B, B′, and B″ show staining for GFP. Panels C, C′ and C″ show staining for Sox2. Panels A′, B′ and C′ represent a higher magnification of the corresponding area in the square in panels A, B and C. Panels A″, B″ and C″ represent a higher magnification of the corresponding area within the dashed lines in panels A′, B′ and C′. The scale bars represent 100 μm in panels A, B and C; 25 μm in panels A′, A″, B′, B″, C′ and C″.

FIG. 6 is a series of micrographs of a composite section of the organ of Corti showing inner and outer hair cells in an untreated adult mouse stained for actin (left panel) and native stereocilin (right panel). The scale bar represents 10 μm.

FIG. 7 is a series of micrographs from the organ of Corti of an adult mouse administered an AAV vector expressing GFP under control of the STRC promoter of nucleotides 35-530 of SEQ ID NO: 2 derived from plasmid P1209. Panels A and B show composite serial sections from different planes of the organ of Corti oriented left to right from base to apex. Panels A and A′ show staining for Myo7a. Panel A′ is a higher magnification of the area shown in a rectangle in panel A. Panels B and B′ show staining for GFP. Panel B′ is a higher magnification of the area shown in a rectangle in panel B. The scale bars represent 100 μm in panels A and B; 50 μm in panels A′ and B′.

FIG. 8 is a series of micrographs from the organ of Corti of an adult mouse administered an AAV vector expressing GFP under control of the STRC promoter of SEQ ID NO: 1 derived from plasmid P1208. Panels A and B show composite serial sections from different planes of the organ of Corti oriented left to right from base to apex. Panels A, A′ and A″ show staining for Myo7a. Panel A′ is a higher magnification of the area shown in a solid rectangle in panel A. Panel A″ is a higher magnification of the area shown in a dashed square in panel A. Panels B, B′ and B″ show staining for GFP. Panel B′ is a higher magnification of the area shown in a rectangle in panel B. Panel B″ is a higher magnification of the area shown in a dashed square in panel B. The scale bars represent 100 am in panels A and B; 50 μm in panels A′, A″, B′ and B″.

FIG. 9 is a series of micrographs from the organ of Corti of a non-human primate (Macaca fascicularis) administered an AAV vector expressing H2B-GFP under control of the STRC promoter of SEQ ID NO: 1 derived from plasmid P1016. Panels A and B show composite serial sections from different planes in the midturn of the organ of Corti. Panel A shows staining for inner and outer hair cells. Panel B shows antibody staining for GFP. The scale bars represent 50 μm. Inner hair cells (IHCs) and outer hair cells (OHCs) are highlighted for orientation.

FIG. 10 is a micrograph of a single paraffin section from a cochlea mid turn of a non-human primate (Macaca fascicularis) administered an AAV vector expressing H2B-GFP under control of the STRC promoter of SEQ ID NO: 1 derived from plasmid P1016. Panel A shows a grey scale conversion of the area around the organ of Corti with Hematoxylin-stained nuclei originally in blue and H2B-GFP antibody originally stained in red. Panel B shows the remaining signal after removing the signal for blue Hematoxylin; H2B-GFP positive (red) nuclei remain visible as darker colors after greyscale conversion. The scale bars represent 100 μm. Inner hair cells (IHCs) and outer hair cells (OHCs) are highlighted for orientation.

FIG. 11 is a plasmid map of plasmid P1016.

FIGS. 12A-12C are a series of fluorescent images of stereocilin expression in the mouse organ of Corti in a 200 μm² ROI at 16 kHz. FIG. 12A shows stereocilin antibody staining at the tips of the outer hair cell (OHC) stereocilia in a wild-type CBA/CaJ mouse. As shown in FIG. 12B, 232 bp STRC knockout (KO) animals lacked the signal for the antibody. FIG. 12C shows stereocilin antibody staining in a 232 bp STRC KO mouse administered dual Anc80 vectors, in which the first vector carried a CMV promoter and nucleotides 1-3200 of the murine STRC cDNA and the vector second carried nucleotides 2201-5430, creating a 1000 bp overlap between the two cDNA in the two vectors. De-novo stereocilin protein expression could be observed at the tips of the OHC stereocilia and in the body of inner hair cells of the organ of Corti in treated 232 bp STRC KO mice.

FIGS. 13A-13C are a series of graphs showing improvement of auditory function in Anc80-CMV-mStrc treated 232 bp STRC KO mice and correlation to OHC STRC expression. Untreated contralateral ears showed near absent DPOAEs and highly elevated ABR thresholds indicative of loss of OHC function (FIGS. 13A-13B, open circles), while treated 232 bp STRC KO animals showed recovery of hearing thresholds (FIGS. 13A-13B, filled circles). The best responder of the treated animals (FIGS. 13A-13B, black squares) showed close to wild-type (FIGS. 13A-13B, triangles) hearing thresholds. A high fraction of OHCs of 232 bp STRC KO mice expressing stereocilin after treatment with AAV-Anc80-CMV-mStrc was found to promote hearing recovery (FIG. 13C).

FIGS. 14A-14B are an image and a graph showing that transfection of HEK293T cells with a two-vector split intein system led to reconstitution of full-length stereocilin. FIG. 14A is a representative image of a Western blot against stereocilin protein and beta-actin. FIG. 14B is a densitometry quantification of full-length stereocilin band intensity relative to actin and indicates the relative expression of full-length stereocilin protein for the negative (GFP) control, positive full-length control, and the Npu intein construct.

FIG. 15 is a graph showing stereocilin protein expression levels detected using a single plasmid vector or an AAV dual vector system in HEK293T cells. Full-length control was plasmid DNA containing the full murine STRC coding sequence. GFP was also expressed using a plasmid. The AAV dual hybrid vectors tested differed in the split site used to divide the murine STRC sequence between the first and second vectors. The value on the x-axis indicates the final nucleotide of murine STRC in the 5′ vector (e.g., Dual hybrid 1800 is a dual hybrid vector system in which the 5′ vector contained nucleotides 1-1800 of murine STRC, with the remaining nucleotides of the STRC coding sequence starting at nucleotide 1801 contained in the 3′ vector). Overlapping was an overlapping dual AAV vector system in which the 5′ and 3′ vectors shared 1,000 nucleotides in common from the STRC coding sequence (the 5′ vector carried nucleotides 79-3278 of NM_080459 (corresponding to nucleotides 1-3200 of SEQ ID NO: 6) and the 3′ vector carried nucleotides 2279-5508 (corresponding to nucleotides 2201-5430 of SEQ ID NO: 6)).

FIGS. 16A-16B are a series of micrographs taken from mouse neonatal cochlear explants infected with AAV vectors expressing enhanced GFP under control of various STRC promoters. The top row shows staining for hair cell marker Myo7a and GFP. The middle row shows only Myo7a staining. The bottom row shows only GFP staining. Control micrographs were obtained from untreated (no AAV infection) explants.

FIG. 17 is a bar graph quantifying the percent of hair cells that were positive for GFP in mouse neonatal cochlear explants infected with AAV vectors expressing enhanced GFP under control of various STRC promoters.

FIG. 18 is a bar graph separately quantifying the percent of inner and outer hair cells that were positive for GFP in mouse neonatal cochlear explants infected with AAV vectors expressing enhanced GFP under control of various STRC promoters.

### DETAILED DESCRIPTION

Described herein are compositions and methods for inducing transgene expression specifically in hair cells (e.g., cochlear and vestibular hair cells). The invention features STRC promoters that are capable of inducing expression of an expression product (e.g., a protein encoded by a transgene or an RNA molecule, such as an inhibitory RNA molecule) in hair cells (e.g., cochlear hair cells, such as outer and inner hair cells, and vestibular hair cells, such as type I and type II vestibular hair cells) that endogenously express STRC. The invention also features nucleic acid vectors containing such promoters operably linked to a polynucleotide encoding an expression product (e.g., a polynucleotide encoding a protein or an inhibitory RNA). The compositions and methods described herein can be used to express a desired expression product (e.g., a protein, inhibitory RNA, microRNA, or a component of a gene editing system) specifically in hair cells, and, therefore, the compositions described herein can be administered to a subject (such as a mammalian subject, for instance, a human) to treat disorders caused by dysfunction of cochlear or vestibular hair cells, such as hearing loss and vestibular dysfunction.

### Hair Cells

Hair cells are sensory cells of the auditory and vestibular systems that reside in the inner ear. Cochlear hair cells are the sensory cells of the auditory system and are made up of two main cell types: inner hair cells, which are responsible for sensing sound, and outer hair cells, which are thought to amplify low-level sound. Vestibular hair cells are located in the semicircular canal end organs and otolith organs of the inner ear and are involved in the sensation of movement that contributes to the sense of balance and spatial orientation. Hair cells are named for the stereocilia that protrude from the apical surface of the cell, forming a hair cell bundle. Deflection of the stereocilia (e.g., by sound waves in cochlear hair cells, or by rotation or linear acceleration in vestibular hair cells) leads to the opening of mechanically gated ion channels, which allows hair cells to release neurotransmitters to activate nerves, thereby converting mechanical sound or motion signals into electrical signals that can be transmitted to the brain. Cochlear hair cells are essential for normal hearing, and damage to or loss of cochlear hair cells and genetic mutations that disrupt cochlear hair cell function are implicated in hearing loss and deafness. Damage to or loss of vestibular hair cells and genetic mutations that disrupt vestibular hair cell function are implicated in vestibular dysfunction, such as dizziness, vertigo, balance loss, bilateral vestibulopathy (also known as bilateral vestibular hypofunction), oscillopsia, and balance disorders. Gene therapy has recently emerged as an attractive therapeutic approach for treating hearing loss and vestibular dysfunction; however, the field is in need of methods to specifically target the nucleic acid vectors used in gene therapy to hair cells.

The present invention is based, in part, on the discovery of STRC promoter sequences that can be used to induce gene expression in hair cells that endogenously express STRC (e.g., cochlear and vestibular hair cells). These STRC promoters are hair cell-specific promoters. The compositions and methods described herein can, thus, be used to express a desired expression product in hair cells, such as a gene implicated in hair cell development, hair cell function, hair cell fate specification, hair cell regeneration, hair cell survival, or hair cell maintenance, or a gene known to be disrupted, e.g., mutated, in subjects with hearing loss or vestibular dysfunction, to treat subjects having or at risk of developing hearing loss (e.g., sensorineural hearing loss), tinnitus, or vestibular dysfunction (e.g., dizziness, vertigo, imbalance, bilateral vestibulopathy, oscillopsia, or a balance disorder).

### Stereocilin

Stereocilin (also known as DFNB16) is a protein encoded by the STRC gene on chromosome 15q15, which contains 29 exons spanning approximately 19 kb of the genome. The STRC gene is tandemly duplicated, where the second copy contains a premature stop codon in exon 20, thereby producing an STRC pseudogene. Previous studies have identified two frameshift mutations and a large deletion in the full-length copy of STRC in two families with autosomal recessive non-syndromic sensorineural hearing loss (Verpy et al., Nat. Genet. 29:345-9 (2001)). Stereocilin protein expression is limited to stereocilia in hair bundles of hair cells and stereocilin is thought to form horizontal top connectors and tectorial membrane-attachment crowns, which are required for the normal functioning of the auditory apparatus (Avan et al., PNAS 116:25948-57 (2019); Verpy et al., J. Comp. Neurol. 519:194-210 (2011)). Mice lacking stereocilin have been shown to exhibit abnormal hair cell bundles with defective cohesion and impaired hearing (Verpy et al., Nature 456:255-8 (2008)).

The present invention is based, in part, on the discovery of a 500 base pair (bp) region located upstream of the human STRC translation start site and a 537 bp region located upstream of the mouse STRC translation start site that can be used to induce gene expression in hair cells that endogenously express STRC. The compositions and methods described herein can, thus, be used to express a desired expression product in hair cells (e.g., cochlear hair cells, such as outer or inner hair cells, or vestibular hair cells, such as type I or type II hair cells), such as a gene implicated in hair cell development, function, cell fate specification, regeneration, survival, or maintenance, or a gene known to be disrupted, e.g., mutated, in subjects with hearing loss or vestibular dysfunction to treat subjects having or at risk of developing hearing loss (e.g., sensorineural hearing loss) or vestibular dysfunction (e.g., dizziness, vertigo, imbalance, bilateral vestibulopathy, oscillopsia, or a balance disorder). The discovery of the smallest possible STRC promoters that retain endogenous promoter activity is advantageous for use in nucleic acid vectors, particularly for use in vectors in which packaging capacity is limited, such as AAV vectors, which are thought to have a maximum packaging capacity of about 4.7 kb.

The compositions and methods described herein include STRC promoters listed in Table 2 (e.g., SEQ ID NO: 1, SEQ ID NO: 2, or SEQ ID NO: 48) and portions thereof that are capable of expressing a desired expression product (e.g., a transgene) specifically in hair cells that endogenously express STRC (e.g., cochlear and vestibular hair cells), such as polynucleotide sequences that have at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, or SEQ ID NO: 48 or a functional portion of SEQ ID NO: 2 or SEQ ID NO: 48. In some embodiments, the functional portion of SEQ ID NO: 2 includes or has the sequence of nucleotides 252-537 or 35-530 of SEQ ID NO: 2. In some embodiments, the functional portion of SEQ ID NO: 2 is a larger portion of SEQ ID NO: 2 that includes nucleotides 252-537 of SEQ ID NO: 2, such as a portion that includes nucleotides 120-537 of SEQ ID NO: 2. In some embodiments, the functional portion of SEQ ID NO: 48 includes or has the sequence of nucleotides 280-560 of SEQ ID NO: 48. In some embodiments, the functional portion of SEQ ID NO: 48 is a larger portion of SEQ ID NO: 48 that includes nucleotides 280-560 of SEQ ID NO: 48, such as a portion that includes nucleotides 280-564 of SEQ ID NO: 48, nucleotides 124-560 of SEQ ID NO: 48, nucleotides 124-564 of SEQ ID NO: 48, or nucleotides 1-560 of SEQ ID NO: 48. In some embodiments, the STRC promoter for use in the compositions and methods described herein includes a portion that has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, nucleotides 252-537 of SEQ ID NO: 2, nucleotides 120-537 of SEQ ID NO:2, nucleotides 35-530 of SEQ ID NO:2, nucleotides 280-560 of SEQ ID NO: 48, nucleotides 280-564 of SEQ ID NO: 48, nucleotides 124-560 of SEQ ID NO: 48, nucleotides 124-564 of SEQ ID NO: 48, or nucleotides 1-560 of SEQ ID NO: 48. In some embodiments, the STRC promoter for use in the compositions and methods described herein has the sequence of SEQ ID NO: 1, SEQ ID NO: 2, SEQ ID NO: 48, nucleotides 252-537 of SEQ ID NO: 2, nucleotides 120-537 of SEQ ID NO:2, nucleotides 35-530 of SEQ ID NO:2, nucleotides 280-560 of SEQ ID NO: 48, nucleotides 280-564 of SEQ ID NO: 48, nucleotides 124-560 of SEQ ID NO: 48, nucleotides 124-564 of SEQ ID NO: 48, or nucleotides 1-560 of SEQ ID NO: 48.

Exemplary STRC promoter sequences are listed in Table 2.

TABLE 2
STRC promoter sequences

| SEQ | Description | |
|---|---|---|
| ID | of promoter | |
| NO: | sequence | Promoter sequence |
| 1 | Human STRC | TGAATTCTCCATCCTTGGACTGGACCTCTC |
| | promoter | GTCTGTTTATACCTTGCTGTGCAGTTTGTT |
| | sequence | TCTCCTGGGCGTCATTTCTTCTTCAATTTC |
| | (500 bp) | CTCAAATCCTACTTGCTTCTTAGCTCCTTC |
| | | ATATCCTCTTTGTTTTCTTTGCTTCTGCAG |
| | | AACCCCAGATTACACCCCTGGTCTCCAGTT |
| | | CTCCATCTCCATGTCTGCCTGTTCCTTTCT |
| | | GCATCACTGGCTCCTAACTCAAGCAATCTC |
| | | CTTGGGTTTCTCTGAGGGGCCCCTGGGATC |
| | | CCCTATCATTAGTCCCTCTCACAGAAGCAT |
| | | ACCCTTCTCCAGAGCTAAAGGATCAGATAT |
| | | TCAGCGGCTCAGGTAACAAACCTGCTGTCA |
| | | GGTTACACATATTGTTTCCTGAAAGACCAC |
| | | ACTACAGTGTCAGTGGAGCCTCAGGTTGCC |
| | | TGCAGTGCCCTGCCCTCACCTGGCTATCCC |
| | | ACACAGGTGAGAATAACCAGAACTCACCTC |
| | | CGGTACCAGTGTTCACTTGG |
| 2 | Murine STRC | GCCATCTCTTCAGCACCAATACTGTGGGAC |
| | promoter | TCTGGATAGTGTGTCTAAGGTAACAACTCT |
| | sequence | CCTCTTCCTAAATAGTCTACCCTTGAAACA |
| | (537 bp) | TCTATCTTAATATCTTCTCGCTGCCCACTG |
| | | CTGGCTGCCAGACCATTTCTCAAATTCTTC |
| | | AAATCTCAGTTGTCTCTCAAACTCCTTTTG |
| | | TACTTTCTTTGTTTGATCACATTTTTCTGT |
| | | AGACCCCCAGATTATATTTTTAGTCTGCCT |
| | | TCTTCTCTCTGGCTCTTAACTCATGCCGTC |
| | | TCCTTGGGTAATACTCTGAGGGCCCCGAGA |
| | | TCCCCTGTCATTGGTCCCTCTAACAGAAGT |
| | | ACACCCTTCTCCCAAAGCCAGAGGATCAGG |
| | | TGTTCAGTGGCTCCGGTGACAGGTGTGATG |
| | | TCAGGTTACAGATACTGTTTCCTGAGAGAC |
| | | CACACGACAGTGTCAGAGGAACCCCAGCCT |
| | | GCCCACAATGCCCCGTCTTCACCTGGCTAT |
| | | CCCTTCATGGTGAGCATAGCCAGAACTCAC |
| | | CTCTAGGCCCAGTGTGCACCTGGAAAT |
| 48 | Human STRC | CTGCTCAGCTCTTGACACTCTGTGGTTCTG |
| | promoter | GGTAGTAGTTGAAAGCAGCTCTCCTCTTCC |
| | sequence | TGAATTCTCCATCCTTGGACTGGACCTCTC |
| | (564 bp) | GTCTGTTTATACCTTGCTGTGCAGTTTGTT |
| | | TCTCCTGGGCGTCATTTCTTCTTCAATTTC |
| | | CTCAAATCCTACTTGCTTCTTAGCTCCTTC |
| | | ATATCCTCTTTGTTTTCTTTGCTTCTGCAG |
| | | AACCCCAGATTACACCCCTGGTCTCCAGTT |
| | | CTCCATCTCCATGTCTGCCTGTTCCTTTCT |
| | | GCATCACTGGCTCCTAACTCAAGCAATCTC |
| | | CTTGGGTTTCTCTGAGGGGCCCCTGGGATC |
| | | CCCTATCATTAGTCCCTCTCACAGAAGCAT |
| | | ACCCTTCTCCAGAGCTAAAGGATCAGATAT |
| | | TCAGCGGCTCAGGTAACAAACCTGCTGTCA |
| | | GGTTACACATATTGTTTCCTGAAAGACCAC |
| | | ACTACAGTGTCAGTGGAGCCTCAGGTTGCC |
| | | TGCAGTGCCCTGCCCTCACCTGGCTATCCC |
| | | ACACAGGTGAGAATAACCAGAACTCACCTC |
| | | CGGTACCAGTGTTCACTTGGAAAC |

The foregoing promoter sequences can be included in a nucleic acid vector and operably linked to a polynucleotide encoding a desired expression product (e.g., a polynucleotide encoding a protein of interest or an inhibitory RNA) to express the expression product specifically in hair cells (e.g., in hair cells that endogenously express STRC, such as cochlear hair cells (e.g., outer hair cells and inner hair cells) and vestibular hair cells (e.g., type I and type II vestibular hair cells)). In some embodiments, the polynucleotide operably linked to the STRC promoter is a transgene that encodes a protein implicated in hair cell function, hair cell development, hair cell fate specification, hair cell regeneration, hair cell survival, or hair cell maintenance, or a transgene corresponding to the wild-type version of a gene that has been found to be mutated in subjects having hearing loss, deafness, auditory neuropathy, tinnitus, or vestibular dysfunction (e.g., dizziness, vertigo, imbalance, bilateral vestibulopathy, oscillopsia, or a balance disorder). According to the methods described herein, a subject can be administered a composition containing one or more of the foregoing polynucleotides (e.g., an STRC promoter, e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide encoding a desired expression product, such as a transgene encoding a protein of interest. In some embodiments, the protein encoded by the transgene is Actin Gamma 1 (ACTG1), Fascin Actin-Bundling Protein 2, Retinal (FSCN2), Radixin (RDX), POU Class 4 Homeobox 3 (POU4F3), TRIO and F-Actin Binding Protein (TRIOBP), Taperin (TPRN), Xin Actin Binding Repeat Containing 2 (XIRP2), Atonal BHLH Transcription Factor 1 (ATOH1), Growth Factor Independent 1 Transcriptional Repressor (GFI1), Cholinergic Receptor Nicotinic Alpha 9 Subunit (CHRNA9), Cholinergic Receptor Nicotinic Alpha 10 Subunit (CHRNA10), Calcium and Integrin Binding Family Member 3 (CIB3), Cadherin 23 (CDH23), Protocadherin 15 (PCDH15), Kinocilin (KNCN), Pejvakin (DFNB59), MKRN2 Opposite Strand (MKRN2OS), LIM Homeobox Protein 3 (LHX3), Transmembrane Channel Like 1 (TMC1), Myosin 15 (MYO15), Myosin 7A (MYO7A), Myosin 6 (MYO6), Myosin IIIA (MYO3A), Myosin IIIB (MYO3B), Glutaredoxin Domain Containing Cysteine-Rich Protein 1 (GRXCR1), Protein Tyrosine Phosphatase, Receptor Type Q (PTPRQ), Late Cornified Envelope 6A (LCE6A), Lipoxygenase Homology Domain-containing Protein 1 (LOXHD1), ADP-Ribosyltransferase 1 (ART1), ATPase Plasma Membrane Ca2+ Transporting 2 (ATP2B2), Calcium and Integrin Binding Family Member 2 (CIB2), Calcium Voltage-Gated Channel Auxiliary Subunit Alpha2delta 4 (CACNA2D4), Epidermal Growth Factor Receptor Pathway Substrate 8 (EPS8), EPS8 Like 2 (EPS8L2), Espin (ESPN), Espin Like (ESPNL), Peripherin 2 (PRPH2), Solute Carrier Family 8 Member A2 (SLC8A2), Zinc Finger CCHC-Type Containing Protein 12 (ZCCHC12), Leucine Rich Transmembrane and O-methyltransferase Domain Containing (LRTOMT2, LRTOMT1), USH1 Protein Network Component Harmonin (USH1C), Solute Carrier Family 26 Member 5 (SLC26A5), Piezo Type Mechanosensitive Ion Channel Component 2 (PIEZO2), Extracellular Leucine Rich Repeat and Fibronectin Type III Domain Containing 1 (ELFN1), Tetratricopeptide Repeat Protein 24 (TTC24), Dystrotelin (DYTN), Coiled-coil Glutamate Rich Protein 2 (CCER2), Leucine-rich Repeat and Transmembrane Domain-containing protein 2 (LRTM2), Potassium Voltage-Gated Channel Subfamily A Member 10 (KCNA10), Clarin 1 (CLRN1), Clarin 2 (CLRN2), SKI Family Transcriptional Corepressor 1 (SKOR1), Tctexl Domain Containing Protein 1 (TCTEX1 D1), Fc Receptor Like B (FCRLB), Glutaredoxin Domain Containing Cysteine-Rich Protein 2 (GRXCR2), Serpin Family E Member 3 (SERPINE3), Nescient Helix-loop Helix 1 (NHLH1), Heat Shock Protein 70 (HSP70), Heat Shock Protein 90 (HSP90), Activating Transcription Factor 6 (ATF6), Eukaryotic Translation Initiation Factor 2 Alpha Kinase 3 (PERK), Serine/Threonine-Protein Kinase/Endoribonuclease IRE1 (IRE1), Whirlin (WHRN), Oncomodulin (OCM), LIM Homeobox 1 (Isl1), Transmembrane and Tetratricopeptide Repeat Containing 4 (TMTC4), or Binding Immunoglobulin Protein (BIP), or Potassium Voltage-Gated Channel Subfamily Q Member 4 (KCNQ4).

In some embodiments, an STRC promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) is operably linked to a polynucleotide encoding an N-terminal portion of stereocilin (e.g., a polynucleotide encoding an N-terminal portion of SEQ ID NO: 3 or SEQ ID NO: 4) and incorporated into a first vector in a two-vector system. The full-length stereocilin coding sequence is too large to include in the type of vector that is commonly used for gene therapy (e.g., an AAV vector), but this problem can be solved by dividing the stereocilin coding sequence between two different nucleic acid vectors such that the full-length stereocilin sequence can be reconstituted in a cell (e.g., a hair cell). Such two-vector systems can be used to treat sensorineural hearing loss or vestibular dysfunction in a subject by administering to the inner ear of a subject a first nucleic acid vector containing a polynucleotide encoding an N-terminal portion of a stereocilin protein and a second nucleic acid vector containing a polynucleotide encoding a C-terminal portion of a stereocilin protein. These two-vector systems can be used to treat a subject having one or more mutations in the STRC gene, e.g., a STRC mutation that reduces stereocilin expression, reduces stereocilin function, or is associated with hearing loss or vestibular dysfunction. When the first and second nucleic acid vectors are administered in a composition, the polynucleotides encoding the N-terminal and C-terminal portions of stereocilin can combine within a cell (e.g., a human cell, e.g., a cochlear or vestibular hair cell) to form a single polynucleotide that contains the full-length stereocilin coding sequence (e.g., through homologous recombination and/or splicing).

The nucleic acid vectors in the two-vector systems described herein include polynucleotide sequences that encode WT stereocilin, or a variant thereof, such as polynucleotide sequences that, when combined, encode a protein having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of WT mammalian (e.g., human or mouse) stereocilin. The polynucleotides used in the two-vector systems described herein encode an N-terminal portion and a C-terminal portion of a stereocilin amino acid sequence in Table 3 below (e.g., two portions that, when combined, encode a full-length stereocilin amino acid sequence listed in Table 3, e.g., SEQ ID NO: 3 or SEQ ID NO: 4).

TABLE 3
Stereocilin Protein and Nucleic Acid Sequences

| SEQ ID | | |
|---|---|---|
| NO. | Sequence Name | Sequence |
| 3 | Wild-type human | MALSLWPLLLLLLLLLLLSFAVTLAPTGPH |
| | stereocilin | SLDPGLSFLKSLLSTLDQAPQGSLSRSRFF |
| | protein, | TFLANISSSFEPGRMGEGPVGEPPPLQPPA |
| | UniProt ID: | LRLHDFLVTLRGSPDWEPMLGLLGDMLALL |
| | Q7RTU9 | GQEQTPRDFLVHQAGVLGGLVEVLLGALVP |
| | | GGPPTPTRPPCTRDGPSDCVLAADWLPSLL |
| | | LLLEGTRWQALVQVQPSVDPTNATGLDGRE |
| | | AAPHFLQGLLGLLTPTGELGSKEALWGGLL |
| | | RTVGAPLYAAFQEGLLRVTHSLQDEVFSIL |
| | | GQPEPDTNGQCQGGNLQQLLLWGVRHNLSW |
| | | DVQALGFLSGSPPPPPALLHCLSTGVPLPR |
| | | ASQPSAHISPRQRRAITVEALCENHLGPAP |
| | | PYSISNFSIHLLCQHTKPATPQPHPSTTAI |
| | | CQTAVWYAVSWAPGAQGWLQACHDQFPDEF |
| | | LDAICSNLSFSALSGSNRRLVKRLCAGLLP |
| | | PPTSCPEGLPPVPLTPDIFWGCFLENETLW |
| | | AERLCGEASLQAVPPSNQAWVQHVCQGPTP |
| | | DVTASPPCHIGPCGERCPDGGSFLVMVCAN |
| | | DTMYEVLVPFWPWLAGQCRISRGGNDTCFL |
| | | EGLLGPLLPSLPPLGPSPLCLTPGPFLLGM |
| | | LSQLPRCQSSVPALAHPTRLHYLLRLLTFL |
| | | LGPGAGGAEAQGMLGRALLLSSLPDNCSFW |
| | | DAFRPEGRRSVLRTIGEYLEQDEEQPTPSG |
| | | FEPTVNPSSGISKMELLACFSPVLWDLLQR |
| | | EKSVWALQILVQAYLHMPPENLQQLVLSAE |
| | | REAAQGFLTLMLQGKLQGKLQVPPSEEQAL |
| | | GRLTALLLQRYPRLTSQLFIDLSPLIPFLA |
| | | VSDLMRFPPSLLANDSVLAAIRDYSPGMRP |
| | | EQKEALAKRLLAPELFGEVPAWPQELLWAV |
| | | LPLLPHLPLENFLQLSPHQIQALEDSWPAA |
| | | GLGPGHARHVLRSLVNQSVQDGEEQVRRLG |
| | | PLACFLSPEELQSLVPLSDPTGPVERGLLE |
| | | CAANGTLSPEGRVAYELLGVLRSSGGAVLS |
| | | PRELRVWAPLFSQLGLRFLQELSEPQLRAM |
| | | LPVLQGTSVTPAQAVLLLGRLLPRHDLSLE |
| | | ELCSLHLLLPGLSPQTLQAIPRRVLVGACS |
| | | CLAPELSRLSACQTAALLQTFRVKDGVKNM |
| | | GTTGAGPAVCIPGQPIPTTWPDCLLPLLPL |
| | | KLLQLDSLALLANRRRYWELPWSEQQAQFL |
| | | WKKMQVPTNLTLRNLQALGTLAGGMSCEFL |
| | | QQINSMVDFLEVVHMIYQLPTRVRGSLRAC |
| | | IWAELQRRMAMPEPEWTTVGPELNGLDSKL |
| | | LLDLPIQLMDRLSNESIMLVVELVQRAPEQ |
| | | LLALTPLHQAALAERALQNLAPKETPVSGE |
| | | VLETLGPLVGFLGTESTRQIPLQILLSHLS |
| | | QLQGFCLGETFATELGWLLLQESVLGKPEL |
| | | WSQDEVEQAGRLVFTLSTEAISLIPREALG |
| | | PETLERLLEKQQSWEQSRVGQLCREPQLAA |
| | | KKAALVAGVVRPAAEDLPEPVPNCADVRGT |
| | | FPAAWSATQIAEMELSDFEDCLTLFAGDPG |
| | | LGPEELRAAMGKAKQLWGPPRGFRPEQILQ |
| | | LGRLLIGLGDRELQELILVDWGVLSTLGQI |
| | | DGWSTTQLRIVVSSFLRQSGRHVSHLDFVH |
| | | LTALGYTLCGLRPEELQHISSWEFSQAALF |
| | | LGTLHLQCSEEQLEVLAHLLVLPGGFGPIS |
| | | NWGPEIFTEIGTIAAGIPDLALSALLRGQI |
| | | QGVTPLAISVIPPPKFAVVFSPIQLSSLTS |
| | | AQAVAVTPEQMAFLSPEQRRAVAWAQHEGK |
| | | ESPEQQGRSTAWGLQDWSRPSWSLVLTISF |
| | | LGHLL |
| 4 | Murine | MALSLQPQLLLLLSLLPQEVTSAPTGPQSL |
| | stereocilin | DAGLSLLKSFVATLDQAPQRSLSQSRFSAF |
| | protein | LANISSSFQLGRMGEGPVGEPPPLQPPALR |
| | (NP_536707.2) | LHDFLVTLRGSPDWEPMLGLLGDVLALLGQ |
| | | EQTPRDFLVHQAGVLGGLVEALLGALVPGG |
| | | PPAPTRPPCTRDGPSDCVLAADWLPSLMLL |
| | | LEGTRWQALVQLQPSVDPTNATGLDGREPA |
| | | PHFLQGLLGLLTPAGELGSEEALWGGLLRT |
| | | VGAPLYAAFQEGLLRVTHSLQDEVFSIMGQ |
| | | PEPDASGQCQGGNLQQLLLWGMRNNLSWDA |
| | | RALGFLSGSPPPPPALLHCLSRGVPLPRAS |
| | | QPAAHISPRQRRAISVEALCENHSGPEPPY |
| | | SISNFSIYLLCQHIKPATPRPPPTTPRPPP |
| | | TTPQPPPTTTQPIPDTTQPPPVTPRPPPTT |
| | | PQPPPSTAVICQTAVWYAVSWAPGARGWLQ |
| | | ACHDQFPDQFLDMICGNLSFSALSGPSRPL |
| | | VKQLCAGLLPPPTSCPPGLIPVPLTPEIFW |
| | | GCFLENETLWAERLCVEDSLQAVPPRNQAW |
| | | VQHVCRGPTLDATDFPPCRVGPCGERCPDG |
| | | GSFLLMVCANDTLYEALVPFWAWLAGQCRI |
| | | SRGGNDTCFLEGMLGPLLPSLPPLGPSPLC |
| | | LAPGPFLLGMLSQLPRCQSSVPALAHPTRL |
| | | HYLLRLLTFLLGPGTGGAETQGMLGQALLL |
| | | SSLPDNCSFWDAFRPEGRRSVLRTVGEYLQ |
| | | REEPTPPGLDSSLSLGSGMSKMELLSCFSP |
| | | VLWDLLQREKSVWALRTLVKAYLRMPPEDL |
| | | QQLVLSAEMEAAQGFLTLMLRSWAKLKVQP |
| | | SEEQAMGRLTALLLQRYPRLTSQLFIDMSP |
| | | LIPFLAVPDLMRFPPSLLANDSVLAAIRDH |
| | | SSGMKPEQKEALAKRLLAPELFGEVPDWPQ |
| | | ELLWAALPLLPHLPLESFLQLSPHQIQALE |
| | | DSWPVADLGPGHARHVLRSLVNQSMEDGEE |
| | | QVLRLGSLACFLSPEELQSLVPLSDPMGPV |
| | | EQGLLECAANGTLSPEGRVAYELLGVLRSS |
| | | GGTVLSPRELRVWAPLFPQLGLRFLQELSE |
| | | TQLRAMLPALQGASVTPAQAVLLFGRLLPK |
| | | HDLSLEELCSLHPLLPGLSPQTLQAIPKRV |
| | | LVGACSCLGPELSRLSACQIAALLQTFRVK |
| | | DGV |
| 5 | Polynucleotide | ATGGCTCTCAGCCTCTGGCCCCTGCTGCTG |
| | encoding | CTGCTGCTGCTGCTGCTGCTGCTGTCCTTT |
| | full-length | GCAGTGACTCTGGCCCCTACTGGGCCTCAT |
| | WT human | TCCCTGGACCCTGGTCTCTCCTTCCTGAAG |
| | stereocilin | TCATTGCTCTCCACTCTGGACCAGGCTCCC |
| | (from | CAGGGCTCCCTGAGCCGCTCACGGTTCTTT |
| | NM_153700.2), | ACATTCCTGGCCAACATTTCTTCTTCCTTT |
| | encodes | GAGCCTGGGAGAATGGGGGAAGGACCAGTA |
| | the protein | GGAGAGCCCCCACCTCTCCAGCCGCCTGCT |
| | of SEQ ID NO: 3 | CTGCGGCTCCATGATTTTCTAGTGACACTG |
| | (includes stop | AGAGGTAGCCCCGACTGGGAGCCAATGCTA |
| | codon) | GGGCTGCTAGGGGATATGCTGGCACTGCTG |
| | | GGACAGGAGCAGACTCCCCGAGATTTCCTG |
| | | GTGCACCAGGCAGGGGTGCTGGGTGGACTT |
| | | GTGGAGGTGCTGCTGGGAGCCTTAGTTCCT |
| | | GGGGGCCCCCCTACCCCAACTCGGCCCCCA |
| | | TGCACCCGTGATGGGCCGTCTGACTGTGTC |
| | | CTGGCTGCTGACTGGTTGCCTTCTCTGCTG |
| | | CTGTTGTTAGAGGGCACACGCTGGCAAGCT |
| | | CTGGTGCAGGTGCAGCCCAGTGTGGACCCC |
| | | ACCAATGCCACAGGCCTCGATGGGAGGGAG |
| | | GCAGCTCCTCACTTTTTGCAGGGTCTGTTG |
| | | GGTTTGOTTACCCCAACAGGGGAGCTAGGC |
| | | TCCAAGGAGGCTCTTTGGGGCGGTCTGCTA |
| | | CGCACAGTGGGGGCCCCCCTCTATGCTGCC |
| | | TTTCAGGAGGGGCTGCTCCGTGTCACTCAC |
| | | TCCCTGCAGGATGAGGTCTTCTCCATTTTG |
| | | GGGCAGCCAGAGCCTGATACCAATGGGCAG |
| | | TGCCAGGGAGGTAACCTTCAACAGCTGCTC |
| | | TTATGGGGCGTCCGGCACAACCTTTCCTGG |
| | | GATGTCCAGGCGCTGGGCTTTCTGTCTGGA |
| | | TCACCACCCCCACCCCCTGCCCTCCTTCAC |
| | | TGCCTGAGCACGGGCGTGCCTCTGCCCAGA |
| | | GCTTCTCAGCCGTCAGCCCACATCAGCCCA |
| | | CGCCAACGGCGAGCCATCACTGTGGAGGCC |
| | | CTCTGTGAGAACCACTTAGGCCCAGCACCA |
| | | CCCTACAGCATTTCCAACTTCTCCATCCAC |
| | | TTGCTCTGCCAGCACACCAAGCCTGCCACT |
| | | CCACAGCCCCATCCCAGCACCACTGCCATC |
| | | TGCCAGACAGCTGTGTGGTATGCAGTGTCC |
| | | TGGGCACCAGGTGCCCAAGGCTGGCTACAG |
| | | GCCTGCCACGACCAGTTTCCTGATGAGTTT |
| | | TTGGATGCGATCTGCAGTAACCTCTCCTTT |
| | | TCAGCCCTGTCTGGCTCCAACCGCCGCCTG |
| | | GTGAAGCGGCTCTGTGCTGGCCTGCTCCCA |
| | | CCCCCTACCAGCTGCCCTGAAGGCCTGCCC |
| | | CCTGTTCCCCTCACCCCAGACATCTTTTGG |
| | | GGCTGCTTCTTGGAGAATGAGACTCTGTGG |
| | | GCTGAGCGACTGTGTGGGGAGGCAAGTCTA |
| | | CAGGCTGTGCCCCCCAGCAACCAGGCTTGG |
| | | GTCCAGCATGTGTGCCAGGGCCCCACCCCA |
| | | GATGTCACTGCCTCCCCACCATGCCACATT |
| | | GGACCCTGTGGGGAACGCTGCCCGGATGGG |
| | | GGCAGCTTCCTGGTGATGGTCTGTGCCAAT |
| | | GACACCATGTATGAGGTCCTGGTGCCCTTC |
| | | TGGCCTTGGCTAGCAGGCCAATGCAGGATA |
| | | AGTCGTGGGGGCAATGACACTTGCTTCCTA |
| | | GAAGGGCTGCTGGGCCCCCTTCTGCCCTCT |
| | | CTGCCACCACTGGGACCATCCCCACTCTGT |
| | | CTGACCCCTGGCCCCTTCCTCCTTGGCATG |
| | | CTATCCCAGTTGCCACGCTGTCAGTCCTCT |
| | | GTCCCAGCTCTTGCTCACCCCACACGCCTA |
| | | CACTATCTCCTCCGCCTGCTGACCTTCCTC |
| | | TTGGGTCCAGGGGCTGGGGGCGCTGAGGCC |
| | | CAGGGGATGCTGGGTCGGGCCCTACTGCTC |
| | | TCCAGTCTCCCAGACAACTGCTCCTTCTGG |
| | | GATGCCTTTCGCCCAGAGGGCCGGCGCAGT |
| | | GTGCTACGGACGATTGGGGAATACCTGGAA |
| | | CAAGATGAGGAGCAGCCAACCCCATCAGGC |
| | | TTTGAACCCACTGTCAACCCCAGCTCTGGT |
| | | ATAAGCAAGATGGAGCTGCTGGCCTGCTTT |
| | | AGTCCTGTGCTGTGGGATCTGCTCCAGAGG |
| | | GAAAAGAGTGTTTGGGCCCTGCAGATTCTA |
| | | GTGCAGGCGTACCTGCATATGCCCCCAGAA |
| | | AACCTCCAGCAGCTGGTGCTTTCAGCAGAG |
| | | AGGGAGGCTGCACAGGGCTTCCTGACACTC |
| | | ATGCTGCAGGGGAAGCTGCAGGGGAAGCTG |
| | | CAGGTACCACCATCCGAGGAGCAGGCCCTG |
| | | GGTCGCCTGACAGCCCTGCTGCTCCAGCGG |
| | | TACCCACGCCTCACCTCCCAGCTCTTCATT |
| | | GACCTGTCACCACTCATCCCTTTCTTGGCT |
| | | GTCTCTGACCTGATGCGCTTCCCACCATCC |
| | | CTGTTAGCCAACGACAGTGTCCTGGCTGCC |
| | | ATCCGGGATTACAGCCCAGGAATGAGGCCT |
| | | GAACAGAAGGAGGCTCTGGCAAAGCGACTG |
| | | CTGGCCCCTGAACTGTTTGGGGAAGTGCCT |
| | | GCCTGGCCCCAGGAGCTGCTGTGGGCAGTG |
| | | CTGCCCCTGCTCCCCCACCTCCCTCTGGAG |
| | | AACTTTTTGCAGCTCAGCCCTCACCAGATC |
| | | CAGGCCCTGGAGGATAGCTGGCCAGCAGCA |
| | | GGTCTGGGGCCAGGGCATGCCCGCCATGTG |
| | | CTGCGCAGCCTGGTAAACCAGAGTGTCCAG |
| | | GATGGTGAGGAGCAGGTACGCAGGCTTGGG |
| | | CCCCTCGCCTGTTTCCTGAGCCCTGAGGAG |
| | | CTGCAGAGCCTAGTGCCCCTGAGTGATCCA |
| | | ACGGGGCCAGTAGAACGGGGGCTGCTGGAA |
| | | TGTGCAGCCAATGGGACCCTCAGCCCAGAA |
| | | GGACGGGTGGCATATGAACTTCTGGGTGTG |
| | | TTGCGCTCATCTGGAGGAGCGGTGCTGAGC |
| | | CCCCGGGAGCTGCGGGTCTGGGCCCCTCTC |
| | | TTCTCTCAGCTGGGCCTCCGCTTCCTTCAG |
| | | GAGCTGTCAGAGCCCCAGCTTAGAGCCATG |
| | | CTTCCTGTCCTGCAGGGAACTAGTGTTACA |
| | | CCTGCTCAGGCTGTCCTGCTGCTTGGACGG |
| | | CTCCTTCCTAGGCACGATCTATCCCTGGAG |
| | | GAACTCTGCTCCTTGCACCTTCTGCTACCA |
| | | GGCCTCAGCCCCCAGACACTCCAGGCCATC |
| | | CCTAGGCGAGTCCTGGTCGGGGCTTGTTCC |
| | | TGCCTGGCCCCTGAACTGTCACGCCTCTCA |
| | | GCCTGCCAGACCGCAGCACTGCTGCAGACC |
| | | TTTCGGGTTAAAGATGGTGTTAAAAATATG |
| | | GGTACAACAGGTGCTGGTCCAGCTGTGTGT |
| | | ATCCCTGGTCAGCCTATTCCCACCACCTGG |
| | | CCAGACTGCCTGCTTCCCCTGCTCCCATTA |
| | | AAGCTGCTACAACTGGATTCCTTGGCTCTT |
| | | CTGGCAAATCGAAGACGCTACTGGGAGCTG |
| | | CCCTGGTCTGAGCAGCAGGCACAGTTTCTC |
| | | TGGAAGAAGATGCAAGTACCCACCAACCTT |
| | | ACCCTCAGGAATCTGCAGGCTCTGGGCACC |
| | | CTGGCAGGAGGCATGTCCTGTGAGTTTCTG |
| | | CAGCAGATCAACTCCATGGTAGACTTCCTT |
| | | GAAGTGGTGCACATGATCTATCAGCTGCCC |
| | | ACTAGAGTTCGAGGGAGCCTGAGGGCCTGT |
| | | ATCTGGGCAGAGCTACAGCGGAGGATGGCA |
| | | ATGCCAGAACCAGAATGGACAACTGTAGGG |
| | | CCAGAACTGAACGGGCTGGATAGCAAGCTA |
| | | CTCCTGGACTTACCGATCCAGTTGATGGAC |
| | | AGACTATCCAATGAATCCATTATGTTGGTG |
| | | GTGGAGCTGGTGCAAAGAGCTCCAGAGCAG |
| | | CTGCTGGCACTGACCCCCCTCCACCAGGCA |
| | | GCCCTGGCAGAGAGGGCACTACAAAACCTG |
| | | GCTCCAAAGGAGACTCCAGTCTCAGGGGAA |
| | | GTGCTGGAGACCTTAGGCCCTTTGGTTGGA |
| | | TTCCTGGGGACAGAGAGCACACGACAGATC |
| | | CCCCTACAGATCCTGCTGTCCCATCTCAGT |
| | | CAGCTGCAAGGCTTCTGCCTAGGAGAGACA |
| | | TTTGCCACAGAGCTGGGATGGCTGCTATTG |
| | | CAGGAGTCTGTTCTTGGGAAACCAGAGTTG |
| | | TGGAGCCAGGATGAAGTAGAGCAAGCTGGA |
| | | CGCCTAGTATTCACTCTGTCTACTGAGGCA |
| | | ATTTCCTTGATCCCCAGGGAGGCCTTGGGT |
| | | CCAGAGACCCTGGAGCGGCTTCTAGAAAAG |
| | | CAGCAGAGCTGGGAGCAGAGCAGAGTTGGA |
| | | CAGCTGTGTAGGGAGCCACAGCTTGCTGCC |
| | | AAGAAAGCAGCCCTGGTAGCAGGGGTGGTG |
| | | CGACCAGCTGCTGAGGATOTTCCAGAACCT |
| | | GTGCCAAATTGTGCAGATGTACGAGGGACA |
| | | TTCCCAGCAGCCTGGTCTGCAACCCAGATT |
| | | GCAGAGATGGAGCTCTCAGACTTTGAGGAC |
| | | TGCCTGACATTATTTGCAGGAGACCCAGGA |
| | | CTTGGGCCTGAGGAACTGCGGGCAGCCATG |
| | | GGCAAAGCAAAACAGTTGTGGGGTCCCCCC |
| | | CGGGGATTTCGTCCTGAGCAGATCCTGCAG |
| | | CTTGGTAGGCTCTTAATAGGTCTAGGAGAT |
| | | CGGGAACTACAGGAGCTGATCCTAGTGGAC |
| | | TGGGGAGTGCTGAGCACCCTGGGGCAGATA |
| | | GATGGCTGGAGCACCACTCAGCTCCGCATT |
| | | GTGGTCTCCAGTTTCCTACGGCAGAGTGGT |
| | | CGGCATGTGAGCCACCTGGACTTCGTTCAT |
| | | CTGACAGCGCTGGGTTATACTCTCTGTGGA |
| | | CTGCGGCCAGAGGAGCTCCAGCACATCAGC |
| | | AGTTGGGAGTTCAGCCAAGCAGCTCTCTTC |
| | | CTCGGCACCCTGCATCTCCAGTGCTCTGAG |
| | | GAACAACTGGAGGTTCTGGCCCACCTACTT |
| | | GTACTGCCTGGTGGGTTTGGCCCAATCAGT |
| | | AACTGGGGGCCTGAGATCTTCACTGAAATT |
| | | GGCACCATAGCAGCTGGGATCCCAGACCTG |
| | | GCTCTTTCAGCACTGCTGCGGGGACAGATC |
| | | CAGGGCGTTACTCCTCTTGCCATTTCTGTC |
| | | ATCCCTCCTCCTAAATTTGCTGTGGTGTTT |
| | | AGTCCCATCCAACTATCTAGTCTCACCAGT |
| | | GCTCAGGCTGTGGCTGTCACTCCTGAGCAA |
| | | ATGGCCTTTCTGAGTCCTGAGCAGCGACGA |
| | | GCAGTTGCATGGGCCCAACATGAGGGAAAG |
| | | GAGAGCCCAGAACAGCAAGGTCGAAGTACA |
| | | GCCTGGGGCCTCCAGGACTGGTCACGACCT |
| | | TCCTGGTCCCTGGTATTGACTATCAGCTTC |
| | | CTTGGCCACCTGCTATGA |
| 6 | Polynucleotide | ATGGCTCTGAGCCTCCAGCCCCAGCTGCTC |
| | encoding full- | CTTCTCCTGTCGCTCCTGCCGCAGGAAGTG |
| | length, | ACTTCAGCCCCTACTGGGCCTCAGTCTTTG |
| | murine wild | GATGCTGGTCTCTCCCTTCTGAAGTCATTC |
| | type stereocilin | GTAGCCACTCTGGACCAAGCTCCTCAGCGT |
| | protein | TCCCTCAGCCAGTCACGGTTCTCTGCGTTC |
| | of SEQ ID | CTGGCCAACATTTCTTCATCCTTCCAGCTT |
| | NO: 4 (from | GGGAGGATGGGGGAGGGACCGGTGGGAGAG |
| | NM_080459.2) | CCCCCACCTCTCCAGCCCCCTGCACTTCGA |
| | (includes stop | CTTCATGATTTCCTCGTGACACTGAGAGGT |
| | codon) | AGCCCAGACTGGGAGCCAATGCTAGGGCTT |
| | | CTGGGAGATGTGCTGGCACTCCTGGGACAG |
| | | GAACAGACTCCCCGGGACTTTTTGGTGCAC |
| | | CAGGCAGGTGTACTGGGTGGACTTGTAGAG |
| | | GCATTGTTGGGAGCGTTAGTTCCTGGAGGC |
| | | CCCCCTGCCCCCACTCGACCCCCATGCACC |
| | | CGTGATGGCCCTTCTGACTGTGTCCTGGCT |
| | | GCTGATTGGTTGCCTTCTCTGATGTTGTTA |
| | | TTAGAGGGTACACGCTGGCAGGCCCTGGTG |
| | | CAGTTGCAGCCCAGTGTGGACCCAACCAAT |
| | | GCCACAGGTCTTGATGGTAGAGAGCCAGCT |
| | | CCTCACTTTTTACAGGGTCTGCTGGGCTTG |
| | | CTTACCCCAGCAGGAGAGTTGGGCTCTGAG |
| | | GAGGCTCTTTGGGGTGGTCTGCTGCGCACA |
| | | GTGGGGGCCCCCCTCTATGCTGCCTTCCAG |
| | | GAGGGGCTACTGCGAGTCACTCATTCTCTG |
| | | CAAGATGAGGTCTTTTCTATTATGGGACAG |
| | | CCAGAGCCTGATGCCAGTGGGCAGTGCCAG |
| | | GGAGGCAACCTTCAACAGCTGCTTTTATGG |
| | | GGCATGCGGAACAACCTTTCTTGGGACGCC |
| | | CGAGCACTGGGTTTTCTATCTGGATCACCA |
| | | CCTCCACCCCCTGCTCTCCTGCACTGCCTG |
| | | AGCAGAGGTGTGCCTCTGCCCAGGGCTTCC |
| | | CAGCCTGCGGCTCACATCAGCCCTCGACAG |
| | | CGGCGAGCCATCTCTGTGGAGGCCCTCTGC |
| | | GAGAACCACTCAGGCCCAGAGCCACCCTAC |
| | | AGCATCTCCAACTTCTCCATCTACTTGCTC |
| | | TGCCAGCACATCAAGCCTGCCACCCCGCGG |
| | | CCCCCTCCTACCACCCCACGGCCTCCTCCT |
| | | ACCACCCCACAGCCCCCTCCTACCACTACA |
| | | CAGCCCATTCCTGACACTACACAGCCCCCT |
| | | CCTGTCACCCCAAGGCCTCCTCCTACCACC |
| | | CCACAACCCCCTCCTAGCACAGCTGTCATC |
| | | TGCCAGACAGCTGTATGGTACGCAGTCTCG |
| | | TGGGCACCAGGTGCCCGAGGTTGGCTCCAA |
| | | GCCTGCCATGATCAGTTTCCTGATCAATTT |
| | | CTGGATATGATCTGCGGCAACCTCTCATTT |
| | | TCAGCCCTGTCTGGCCCCAGTCGTCCTTTG |
| | | GTAAAGCAGCTCTGTGCTGGCTTGCTCCCA |
| | | CCCCCCACTAGCTGTCCACCAGGCCTGATC |
| | | CCTGTGCCCCTCACCCCAGAAATATTCTGG |
| | | GGCTGTTTCCTGGAGAATGAGACACTGTGG |
| | | GCTGAACGGTTGTGTGTGGAGGACAGTCTG |
| | | CAGGCTGTGCCCCCGAGGAACCAGGCTTGG |
| | | GTTCAGCATGTGTGTCGGGGCCCCACCTTG |
| | | GACGCCACTGATTTTCCACCGTGCCGCGTT |
| | | GGACCCTGTGGGGAACGCTGCCCAGATGGG |
| | | GGCAGCTTCCTGCTCATGGTCTGTGCCAAT |
| | | GACACTCTGTATGAAGCCTTGGTTCCCTTC |
| | | TGGGCTTGGCTAGCAGGCCAATGCAGAATT |
| | | AGTCGTGGAGGAAATGATACTTGCTTTCTA |
| | | GAAGGCATGCTGGGCCCCTTGTTGCCCTCT |
| | | CTGCCCCCTCTGGGACCATCCCCACTCTGT |
| | | CTGGCTCCTGGTCCTTTTCTGCTTGGCATG |
| | | TTATCCCAGTTGCCACGCTGTCAGTCCTCC |
| | | GTGCCAGCCCTCGCCCACCCCACGCGCCTA |
| | | CATTACCTCCTGCGCCTACTGACCTTCCTT |
| | | CTGGGTCCAGGGACTGGGGGTGCCGAGACG |
| | | CAGGGGATGTTAGGTCAAGCCCTGCTGCTC |
| | | TCTAGTCTCCCAGACAACTGTTCATTCTGG |
| | | GATGCCTTCCGCCCAGAGGGCCGGAGAAGT |
| | | GTACTGAGGACAGTCGGAGAGTACTTGCAG |
| | | CGGGAAGAGCCAACCCCACCAGGCTTAGAC |
| | | TCCTCCCTCAGCCTCGGCTCTGGTATGAGC |
| | | AAGATGGAGCTTCTGTCCTGCTTCAGTCCT |
| | | GTACTGTGGGATCTACTCCAGAGAGAGAAG |
| | | AGCGTTTGGGCCCTGAGGACCCTGGTGAAG |
| | | GCCTACCTGCGCATGCCTCCAGAAGACCTT |
| | | CAGCAGCTTGTGCTTTCAGCAGAGATGGAG |
| | | GCTGCACAGGGCTTCCTGACGCTCATGCTT |
| | | CGTTCCTGGGCTAAGCTGAAGGTTCAACCA |
| | | TCCGAGGAGCAGGCCATGGGCCGCCTGACA |
| | | GCCTTGCTGCTCCAGCGGTACCCACGCCTC |
| | | ACCTCCCAACTCTTTATCGACATGTCACCG |
| | | CTCATCCCCTTCCTGGCTGTCCCTGACCTC |
| | | ATGCGCTTCCCACCGTCCCTTTTGGCCAAC |
| | | GACAGTGTCCTGGCTGCCATCAGGGATCAC |
| | | AGCTCAGGAATGAAGCCTGAACAGAAGGAG |
| | | GCCCTGGCAAAACGACTGCTGGCCCCTGAG |
| | | CTGTTTGGAGAAGTGCCTGATTGGCCCCAG |
| | | GAGCTGCTGTGGGCAGCCCTGCCTCTGCTT |
| | | CCCCATCTGCCTCTGGAGAGCTTTCTCCAG |
| | | CTCAGCCCTCACCAGATCCAGGCCCTGGAG |
| | | GATAGCTGGCCAGTAGCAGATCTTGGGCCG |
| | | GGACACGCCCGACATGTGCTTCGTAGCCTA |
| | | GTAAACCAGAGCATGGAGGATGGGGAGGAG |
| | | CAGGTGCTCAGGCTTGGGTCCCTCGCCTGT |
| | | TTCCTGAGTCCTGAGGAGCTACAGAGTCTG |
| | | GTGCCCTTGAGTGATCCAATGGGGCCTGTA |
| | | GAACAGGGTCTGCTGGAATGTGCGGCCAAT |
| | | GGGACCCTCAGCCCAGAAGGACGGGTGGCA |
| | | TATGAACTTCTGGGAGTGTTGCGTTCATCT |
| | | GGAGGAACTGTCTTAAGCCCCCGAGAGCTG |
| | | AGGGTCTGGGCACCTCTCTTTCCCCAGCTG |
| | | GGCCTCCGCTTCCTGCAGGAGCTCTCAGAG |
| | | ACCCAGCTTAGAGCCATGCTTCCTGCCCTA |
| | | CAGGGAGCCAGTGTCACACCTGCCCAGGCT |
| | | GTTCTGTTGTTTGGAAGGCTCCTTCCTAAG |
| | | CATGATCTGTCCCTGGAGGAACTCTGCTCC |
| | | CTGCACCCTCTCCTGCCAGGTCTCAGCCCC |
| | | CAGACACTCCAGGCCATCCCTAAGAGAGTT |
| | | CTGGTTGGTGCTTGTTCCTGCCTGGGCCCT |
| | | GAACTGTCAAGGCTTTCAGCTTGCCAGATT |
| | | GCAGCTCTGCTGCAGACCTTTCGGGTAAAA |
| | | GATGGTGTTAAAAATATGGGTGCAGCAGGT |
| | | GCCGGCTCAGCCGTGTGCATTCCTGGGCAG |
| | | CCCACCACTTGGCCAGACTGCCTGCTTCCC |
| | | CTGCTCCCATTAAAGCTGCTACAGCTGGAC |
| | | GCTGCAGCTCTTCTGGCAAACCGAAGACTC |
| | | TATCGGCAGCTGCCTTGGTCTGAGCAACAG |
| | | GCACAGTTTCTCTGGAAGAAAATGCAAGTG |
| | | CCTACCAACCTGAGCCTGAGGAATCTGCAG |
| | | GCTCTGGGCAACTTGGCAGGAGGCATGACC |
| | | TGCGAGTTTCTGCAGCAGATCAGCTCAATG |
| | | GTTGACTTTCTTGATGTGGTACACATGCTC |
| | | TACCAGCTGCCCACTGGTGTTOGAGAGAGC |
| | | CTGCGGGCCTGTATCTGGACAGAGCTACAG |
| | | CGGAGGATGACAATGCCAGAGCCAGAGCTG |
| | | ACCACCCTAGGGCCAGAACTGAGTGAACTT |
| | | GACACAAAGCTACTCCTGGACTTGCCGATC |
| | | CAGCTGATGGACAGATTGTCCAATGATTCC |
| | | ATTATGTTGGTGGTGGAGATGGTCCAAGGC |
| | | GCTCCAGAGCAGCTGCTGGCACTGACCCCA |
| | | CTCCACCAGACAGCCTTGGCAGAGCGAGCA |
| | | CTTAAAAACCTGGCTCCAAAGGAGACCCCA |
| | | ATCTCCAAAGAAGTGCTGGAGACACTGGGC |
| | | CCCTTGGTTGGATTCCTGGGAATAGAGAGC |
| | | ACGCGACGGATCCCTTTACCCATTCTACTG |
| | | TCTCATCTCAGTCAGCTGCAGGGCTTCTGC |
| | | CTAGGAGAGACATTTGCCACAGAGCTGGGA |
| | | TGGCTGCTGTTGCAGGAGCCTGTTCTTGGA |
| | | AAACCAGAATTGTGGAGCCAGGATGAAATA |
| | | GAGCAAGCTGGACGCCTAGTATTCACTCTG |
| | | TCTGCTGAGGCTATTTCCTCGATCCCCAGG |
| | | GAGGCTTTGGGCCCAGAGACACTGGAGAGG |
| | | CTTCTGGGAAAGCATCAAAGCTGGGAGCAG |
| | | AGCAGAGTGGGCCATCTGTGTGGGGAGTCA |
| | | CAGCTTGCCCACAAGAAAGCAGCTCTGGTA |
| | | GCTGGGATTGTGCATCCAGCTGCTGAGGGT |
| | | CTCCAAGAGCCTGTACCAAACTGTGCAGAC |
| | | ATACGGGGAACCTTCCCAGCGGCCTGGTCT |
| | | GCGACACAAATCTCAGAGATGGAACTCTCA |
| | | GACTTTGAAGACTGCCTGTCACTATTTGCT |
| | | GGAGATCCAGGACTTGGTCCTGAGGAACTA |
| | | CGGGCAGCCATGGGCAAGGCCAAGCAGTTG |
| | | TGGGGTCCCCCTCGAGGATTCCGTCCTGAG |
| | | CAGATCTTGCAGCTGGGCCGTCTCCTGATA |
| | | GGTCTAGGAGAACGGGAACTGCAGGAGCTT |
| | | ACCTTGGTGGACTGGGGTGTGCTGAGCAGC |
| | | CTGGGGCAAATAGATGGCTGGAGTTCCATG |
| | | CAGCTCCGAGCCGTGGTCTCCAGTTTCCTA |
| | | AGGCAGAGTGGTCGGCATGTGAGCCACCTG |
| | | GACTTCATTTATCTGACAGCACTGGGTTAC |
| | | ACAGTCTGTGGATTGCGACCAGAGGAGTTA |
| | | CAGCACATCAGCAGTTGGGAGTTTAGCCAA |
| | | GCAGCTCTCTTCCTGGGTAGCTTGCATCTC |
| | | CCGTGCTCTGAGGAACAGCTGGAAGTTCTG |
| | | GCCTATCTCCTTGTGTTGCCTGGTGGCTTT |
| | | GGCCCAGTCAGTAACTGGGGGCCTGAGATC |
| | | TTCACTGAAATTGGCACAATAGCAGCTGGC |
| | | ATCCCAGACCTGGCTCTTTCAGCATTACTG |
| | | CGGGGACAGATCCAAGGCCTGACTCCTCTT |
| | | GCCATTTCTGTCATTCCTGCTCCCAAGTTT |
| | | GCAGTGGTCTTCAACCCCATCCAGTTATCT |
| | | AGTCTCACCAGGGGTCAGGCCGTAGCTGTT |
| | | ACTCCTGAACAGCTGGCCTATCTGAGTCCT |
| | | GAGCAGCGGCGAGCAGTTGCATGGGCCCAA |
| | | CACGAAGGGAAGGAGATCCCAGAGCAGCTG |
| | | GGTCGAAACTCAGCCTGGGGTCTCTACGAC |
| | | TGGTTCCAAGCCTCCTGGGCCCTGGCATTG |
| | | CCCGTCAGCATTTTTGGCCACCTATTATGA |

According to the methods described herein, a subject can be administered a composition containing a first nucleic acid vector and a second nucleic acid vector that contain an N-terminal and C-terminal portion, respectively, of a polynucleotide sequence encoding the amino acid sequence of SEQ ID NO: 3 or SEQ ID NO: 4, or a polynucleotide sequence encoding an amino acid sequence having at least 85% sequence identity (e.g., 85%, 86%, 87%, 88%, 89%, 90%, 91%, 92%, 93%, 94%, 95%, 96%, 97%, 98%, 99%, or more, sequence identity) to the amino acid sequence of SEQ ID NO: 3 or SEQ ID NO: 4, or a polynucleotide sequence encoding an amino acid sequence that contains one or more conservative amino acid substitutions relative to SEQ ID NO: 3 or SEQ ID NO: 4 (e.g., 1, 2, 3, 4, 5, 6, 7, 8, 9, or 10 or more conservative amino acid substitutions), provided that the stereocilin analog encoded retains the therapeutic function of WT stereocilin. In some embodiments, no more than 10% of the amino acids in the N-terminal portion of the stereocilin protein and no more than 10% of the amino acids in the C-terminal portion of the stereocilin protein may be replaced with conservative amino acid substitutions. The stereocilin protein may be encoded by a polynucleotide having the sequence of SEQ ID NO: 5 or SEQ ID NO: 6. The stereocilin protein may also be encoded by a polynucleotide having single nucleotide polymorphisms (SNPs) that have been found to be non-pathogenic in human subjects. The stereocilin protein may be a human stereocilin protein or may be a homolog of the human stereocilin protein from another mammalian species (e.g., mouse, rat, cow, horse, goat, sheep, donkey, cat, dog, rabbit, guinea pig, or other mammal).

In some embodiments, the first nucleic acid vector and the second nucleic acid vector are first and second vectors in a dual vector expression system (e.g., overlapping dual vectors, trans-splicing dual vectors, or dual hybrid vectors). In some embodiments, the first nucleic acid vector and the second nucleic acid vector are first and second vectors in an intein expression system. In some embodiments, the first vector and second vector are first and second vectors in a ribozyme expression system (e.g., a system in which the first nucleic acid vector contains a polynucleotide encoding an N-terminal portion of stereocilin and a 3′ ribozyme and the second vector contains a polynucleotide encoding a C-terminal portion of stereocilin and a 5′ ribozyme). In the ribozyme expression system, the 3′ and 5′ ribozymes can catalyze themselves out of the first and second RNA molecules produced during transcription to generate a 3′ and 5′ end that can be ligated to form an RNA molecule containing the coding region of the first RNA molecule and the coding region of the second RNA molecule. In some embodiments, the 3′ ribozyme is a member of the HDV (Hepatitis Delta Virus) family of ribozymes and the 5′ ribozyme is a member of the HH (Hammerhead) family of ribozymes. Exemplary ribozyme expression systems are described in International Application Publication No. WO2021158964A1, which is incorporated herein by reference.

### Dual Vector Expression Systems

### Overlapping Dual Vectors

One approach for expressing large proteins in mammalian cells (e.g., hair cells) involves the use of overlapping dual vectors. This approach is based on the use of two nucleic acid vectors, each of which contains a portion of a polynucleotide that encodes a protein of interest and has a defined region of sequence overlap with the other portion of the polynucleotide. Homologous recombination can occur at the region of overlap and lead to the formation of a single polynucleotide that encodes the full-length protein of interest (e.g., a stereocilin protein of SEQ ID NO: 3 or SEQ ID NO: 4).

Overlapping dual vectors for use in the methods and compositions described herein contain at least 200 bases (b) of overlapping sequence (e.g., at least at least 200 b, 300 b, 400 b, 500 b, 600 b, 700 b, 800 b, 900 b, 1.0 kilobase (kb), 1.1 kb, 1.2 kb, 1.3 kb, 1.4 kb, 1.5 kb or more of overlapping sequence). The nucleic acid vectors are designed such that the overlapping region is centered at or near a position within the stereocilin-encoding polynucleotide that corresponds to approximately half of the length of the stereocilin-encoding polynucleotide, with an equal amount of overlap on either side of the central position. The center of the overlapping region can also be chosen based on the size of the promoter and the locations of sequence elements of interest in the polynucleotide that encodes stereocilin. In some embodiments, the stereocilin-encoding polynucleotide is split in two halves of approximately equal length with some degree of overlap (e.g., 200 b, 250 b, 300 b, 350 b, 400 b, 450 b, 500 b, 600 b, 700 b, 800 b, 900 b, 1 kb, 1.1 kb, 1.2 kb, 1.3 kb, 1.4 kb, 1.5 kb, or more), in which the 5′ half of the polynucleotide encodes an N-terminal portion of the stereocilin protein and the 3′ half of the polynucleotide encodes a C-terminal portion of the stereocilin protein. The nucleic acid vectors for use in the methods and compositions described herein are also designed such that approximately half of the stereocilin-encoding polynucleotide is contained within each vector (e.g., each vector contains a polynucleotide that encodes approximately half of the stereocilin protein).

In some embodiments, the first nucleic acid vector encodes an N-terminal portion of the stereocilin protein. In some embodiments, the second nucleic acid vector encodes a C-terminal portion of the stereocilin protein. In some embodiments, the stereocilin protein has the sequence of SEQ ID NO: 3 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the stereocilin protein has the sequence of SEQ ID NO: 4 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the polynucleotide that encodes a full-length human stereocilin protein has the sequence of SEQ ID NO: 5 or is a variant thereof having at least 85% (e.g., at least 86%, 87%, 88%, 89%, 90%, 91%, 92%, 93%, 94%, 95%, 96%, 97%, 98%, 99%, or more) sequence identity thereto. In some embodiments, the polynucleotide having at least 85% sequence identity to SEQ ID NO: 5 encodes the stereocilin protein of SEQ ID NO: 3. In some embodiments, the polynucleotide that encodes a full-length murine stereocilin protein has the sequence of SEQ ID NO: 6 or is a variant thereof having at least 85% (e.g., at least 86%, 87%, 88%, 89%, 90%, 91%, 92%, 93%, 94%, 95%, 96%, 97%, 98%, 99%, or more) sequence identity thereto. In some embodiments, the polynucleotide having at least 85% sequence identity to SEQ ID NO: 6 encodes the stereocilin protein of SEQ ID NO: 4.

One exemplary overlapping dual vector system includes a first nucleic acid vector containing a STRC promoter described hereinabove (e.g., a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide encoding an N-terminal portion of a stereocilin protein (e.g., an N-terminal portion of SEQ ID NO: 3 or SEQ ID NO: 4) including 500 bp immediately 3′ of the position selected as the central position; and a second nucleic acid vector containing the C-terminal portion of the polynucleotide encoding the stereocilin protein, which includes 500 bp immediately 5′ of the position selected as the central position, and a poly(A) sequence (e.g., a bovine growth hormone (bGH) poly(A) signal sequence). The nucleic acid vectors can optionally contain STRC untranslated regions (UTRs) that are not part of the STRC promoters disclosed herein. In some embodiments, the STRC promoter is a polynucleotide having the sequence of SEQ ID NO: 1 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the STRC promoter is a polynucleotide having the sequence of SEQ ID NO: 2 or a functional portion thereof, or a variant having at least 85% (e.g., at least 86%, 87%, 88%, 89%, 90%, 91%, 92%, 93%, 94%, 95%, 96%, 97%, 98%, 99%, or more) sequence identity SEQ ID NO: 2 or a functional portion thereof. In some embodiments, the STRC promoter is a functional portion of SEQ ID NO: 2 that is capable of controlling expression of the STRC gene. In some embodiments, the functional portion of SEQ ID NO: 2 includes or has the sequence of nucleotides 252-537 of SEQ ID NO: 2. In some embodiments, the functional portion of SEQ ID NO: 2 includes or has the sequence of nucleotides 120-537 of SEQ ID NO: 2. In some embodiments, the functional portion of SEQ ID NO: 2 includes or has the sequence of nucleotides 35-530 of SEQ ID NO: 2. In some embodiments, the STRC promoter is a polynucleotide having the sequence of SEQ ID NO: 48 or a functional portion thereof, or a variant having at least 85% (e.g., at least 86%, 87%, 88%, 89%, 90%, 91%, 92%, 93%, 94%, 95%, 96%, 97%, 98%, 99%, or more) sequence identity SEQ ID NO: 48 or a functional portion thereof. In some embodiments, the STRC promoter is a functional portion of SEQ ID NO: 48 that is capable of controlling expression of the STRC gene. In some embodiments, the functional portion of SEQ ID NO: 48 includes or has the sequence of nucleotides 280-560 of SEQ ID NO: 48. In some embodiments, the functional portion of SEQ ID NO: 48 includes or has the sequence of nucleotides 280-564 of SEQ ID NO: 48. In some embodiments, the functional portion of SEQ ID NO: 48 includes or has the sequence of nucleotides 124-560 of SEQ ID NO: 48. In some embodiments, the functional portion of SEQ ID NO: 48 includes or has the sequence of nucleotides 124-564 of SEQ ID NO: 48. In some embodiments, the functional portion of SEQ ID NO: 48 includes or has the sequence of nucleotides 1-560 of SEQ ID NO: 48.

### Trans-Splicing Dual Vectors

A second approach for expressing large proteins in mammalian cells involves the use of trans-splicing dual vectors. In this approach, two nucleic acid vectors are used that contain distinct nucleic acid sequences, and the polynucleotide encoding the N-terminal portion of the protein of interest and the polynucleotide encoding the C-terminal portion of the protein of interest do not overlap. Instead, the first nucleic acid vector includes a splice donor sequence 3′ of the polynucleotide encoding the N-terminal portion of the protein of interest, and the second nucleic acid vector includes a splice acceptor sequence 5′ of the polynucleotide encoding the C-terminal portion of the protein of interest. When the first and second nucleic acids are present in the same cell, their ITRs can concatenate, forming a single nucleic acid structure in which the concatenated ITRs are positioned between the splice donor and splice acceptor. Trans-splicing then occurs during transcription, producing a nucleic acid molecule in which the polynucleotides encoding the N-terminal and C-terminal portions of the protein of interest are contiguous, thereby forming the full-length coding sequence.

Trans-splicing dual vectors for use in the methods and compositions described herein are designed such that approximately half of the stereocilin coding sequence is contained within each vector (e.g., each vector contains a polynucleotide that encodes approximately half of the stereocilin protein, as is discussed above). The determination of how to split the polynucleotide sequence between the two nucleic acid vectors is made based on the size of the promoter and the locations of sequence elements of interest in the polynucleotide that encodes the stereocilin protein (e.g., exons of the STRC gene). The first vector in the trans-splicing dual vector system can contain a promoter sequence (e.g., an STRC promoter sequence, e.g., SEQ ID NO: 1, SEQ ID NO: 2, SEQ ID NO: 48, or a functional portion of SEQ ID NO: 2 or SEQ ID NO: 48) 5′ of a polynucleotide encoding an N-terminal portion of a stereocilin protein (e.g., an N-terminal portion of a stereocilin protein of SEQ ID NO: 3 or SEQ ID NO: 4). The nucleic acid vectors can optionally contain STRC UTRs (e.g., one or both of the 5′ and 3′ STRC UTRs, e.g., full-length UTRs) that are not part of the promoters described herein. One exemplary trans-splicing dual vector system for use in the compositions and methods described herein includes a first nucleic acid vector containing a STRC promoter described hereinabove (e.g., a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide encoding an N-terminal portion of a stereocilin protein (e.g., a human stereocilin protein, e.g., an N-terminal portion of SEQ ID NO: 3) and a splice donor sequence 3′ of the polynucleotide sequence; and a second nucleic acid vector containing a splice acceptor sequence 5′ of a polynucleotide encoding a C-terminal portion of the stereocilin protein (e.g., a human stereocilin protein, e.g., a C-terminal portion of SEQ ID NO: 3) and a poly(A) sequence. An alternative trans-splicing dual vector system includes a first nucleic acid vector containing a STRC promoter described hereinabove (e.g., a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide encoding an N-terminal portion of the stereocilin protein (e.g., a murine stereocilin protein, e.g., an N-terminal portion of SEQ ID NO: 4) and a splice donor sequence 3′ of the polynucleotide sequence; and a second nucleic acid vector containing a splice acceptor sequence 5′ of a polynucleotide encoding a C-terminal portion of the stereocilin protein (e.g., a murine stereocilin protein, e.g., a C-terminal portion of SEQ ID NO: 4) and a poly(A) sequence. In some embodiments, the STRC promoter is a polynucleotide having the sequence of SEQ ID NO: 1 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the STRC promoter is a polynucleotide having the sequence of SEQ ID NO: 2 or a functional portion thereof, or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 2 or a functional portion thereof. In some embodiments, the STRC promoter is a functional portion of SEQ ID NO: 2 that is capable of controlling expression of the STRC gene. In some embodiments, the functional portion of SEQ ID NO: 2 includes or has the sequence of nucleotides 252-537 of SEQ ID NO: 2. In some embodiments, the functional portion of SEQ ID NO: 2 includes or has the sequence of nucleotides 120-537 of SEQ ID NO: 2. In some embodiments, the functional portion of SEQ ID NO: 2 includes or has the sequence of nucleotides 35-530 of SEQ ID NO: 2. In some embodiments, the STRC promoter is a polynucleotide having the sequence of SEQ ID NO: 48 or a functional portion thereof, or a variant having at least 85% (e.g., at least 86%, 87%, 88%, 89%, 90%, 91%, 92%, 93%, 94%, 95%, 96%, 97%, 98%, 99%, or more) sequence identity SEQ ID NO: 48 or a functional portion thereof. In some embodiments, the STRC promoter is a functional portion of SEQ ID NO: 48 that is capable of controlling expression of the STRC gene. In some embodiments, the functional portion of SEQ ID NO: 48 includes or has the sequence of nucleotides 280-560 of SEQ ID NO: 48. In some embodiments, the functional portion of SEQ ID NO: 48 includes or has the sequence of nucleotides 280-564 of SEQ ID NO: 48. In some embodiments, the functional portion of SEQ ID NO: 48 includes or has the sequence of nucleotides 124-560 of SEQ ID NO: 48. In some embodiments, the functional portion of SEQ ID NO: 48 includes or has the sequence of nucleotides 124-564 of SEQ ID NO: 48. In some embodiments, the functional portion of SEQ ID NO: 48 includes or has the sequence of nucleotides 1-560 of SEQ ID NO: 48.

These nucleic acid vectors can also contain full-length 5′ and/or 3′ STRC UTRs that are not part of the promoters described herein in the first and second nucleic acid vectors, respectively (e.g., the first nucleic acid vector can contain the 5′ human STRC UTR in dual vector systems encoding human stereocilin, or the 5′ mouse UTR in dual vector systems encoding mouse stereocilin; and the second nucleic acid vector can contain the 3′ human STRC UTR in dual vector systems encoding human stereocilin, or the 3′ mouse STRC UTR in dual vector systems encoding mouse stereocilin). To accommodate a STRC UTR, the stereocilin coding sequence can be divided at a different position than the position used to divide the stereocilin coding sequence in a trans-splicing dual vector system that does not include an STRC UTR (e.g., the stereocilin coding sequence can be divided at a position such that the first vector can accommodate the length of the 5′ UTR, the promoter sequence, and the sequence encoding the N-terminal portion of stereocilin and/or such that the second vector can accommodate the length of the C-terminal portion of stereocilin and the length of the 3′ UTR).

In some embodiments, the stereocilin protein has the sequence of SEQ ID NO: 3 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the stereocilin protein has the sequence of SEQ ID NO: 4 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the polynucleotide that encodes a full-length human stereocilin protein has the sequence of SEQ ID NO: 5 or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 5. In some embodiments, the polynucleotide having at least 85% sequence identity to SEQ ID NO: 5 encodes the stereocilin protein of SEQ ID NO: 3. In some embodiments, the polynucleotide that encodes a full-length murine stereocilin protein has the sequence of SEQ ID NO: 6 or is a variant thereof having at least 85% (e.g., at least 86%, 87%, 88%, 89%, 90%, 91%, 92%, 93%, 94%, 95%, 96%, 97%, 98%, 99%, or more) sequence identity SEQ ID NO: 6. In some embodiments, the polynucleotide having at least 85% sequence identity to SEQ ID NO: 6 encodes the stereocilin protein of SEQ ID NO: 4.

### Dual Hybrid Vectors

A third approach for expressing large proteins in mammalian cells (e.g., hair cells) involves the use of dual hybrid vectors. This approach combines elements of the overlapping dual vector strategy and the trans-splicing strategy in that it features both an overlapping region at which homologous recombination can occur and splice donor and splice acceptor sequences. In dual hybrid vector systems, the overlapping region is a recombinogenic region that is contained in both the first and second nucleic acid vectors, rather than a portion of the polynucleotide sequence encoding the protein of interest—the polynucleotide encoding the N-terminal portion of the protein of interest and the polynucleotide encoding the C-terminal portion of the protein of interest do not overlap in this approach. The recombinogenic region is 3′ of the splice donor sequence in the first nucleic acid vector and 5′ of the splice acceptor sequence in the second nucleic acid sequence. The first and second nucleic acid sequences can then join to form a single sequence based on one of two mechanisms: (1) recombination at the overlapping region; or (2) concatemerization of the ITRs. The remaining recombinogenic region(s) and/or the concatemerized ITRs can be removed by splicing, leading to the formation of a contiguous polynucleotide sequence that encodes the full-length protein of interest. Recombinogenic regions, splice donor sequences, and splice acceptor sequences that can be used in the compositions and methods described herein include those well-known to one of skill in the art. Exemplary recombinogenic regions include the F1 phage AK gene and alkaline phosphatase (AP) gene fragments as described in U.S. Pat. Nos. 10,494,645 and 8,236,557, which are incorporated herein by reference. In some embodiments, the AP gene fragment has the sequence of:

(SEQ ID NO: 42)
CCCCGGGTGCGCGGCGTCGGTGGTGCCGGCGGGGGGCGCCAGGTC
GCAGGCGGTGTAGGGCTCCAGGCAGGCGGCGAAGGCCATGACGTG
CGCTATGAAGGTCTGCTCCTGCACGCCGTGAACCAGGTGCGCCTG
CGGGCCGCGCGCGAACACCGCCACGTCCTCGCCTGCGTGGGTCTC
TTCGTCCAGGGGCACTGCTGACTGCTGCCGATACTCGGGGCTCCC
GCTCTCGCTCTCGGTAACATCCGGCCGGGCGCCGTCCTTGAGCAC
ATAGCCTGGACCGTTTCCGTATAGGAGGACCGTGTAGGCCTTCCT
GTCCCGGGCCTTGCCAGCGGCCAGCCCGATGAAGGAGCTCCCTCG
CAGGGGGTAGCCTCCGAAGGAGAAGACGTGGGAGTGGTCGGCAGT
GACGAGGCTCAGCGTGTCCTCCTCGCTGGTGAGCTGGCCCGCCCT
CTCAATGGCGTCGTCGAACATGATCGTCTCAGTCAGTGCCCGGTA
AGCCCTGCTTTCATGATGACCATGGTCGATGCGACCACCCTCCAC
GAAGAGGAAGAAGCCGCGGGGGTGTCTGCTCAGCAGGCGCAGGGC
AGCCTCTGTCATCTCCATCAGGGAGGGGTCCAGTGTGGAGTCTCG
GTGGATCTCGTATTTCATGTCTCCAGGCTCAAAGAGACCCATGAG
ATGGGTCACAGACGGGTCCAGGGAAGCCTGCATGAGCTCAGTGCG
GTTCCACACGTACCGGGCACCCTGGCGTTCGCCGAGCCATTCCTG
CACCAGATTCTTCCCGTCCAGCCTGGTCCCACCTTGGCTGTAGTC
ATCTGGGTACTCAGGGTCTGGGGTTCCCATGCGAAACATGTACTT
TCGGCCTCCA.

In some embodiments, the AP gene fragment has the sequence of:

(SEQ ID NO: 43)
CCCCGGGTGCGCGGCGTCGGTGGTGCCGGGGGGGGCGCCAGGTCG
CAGGCGGTGTAGGGCTCCAGGCAGGCGGCGAAGGCCATGACGTGC
GCTATGAAGGTCTGCTCCTGCACGCCGTGAACCAGGTGCGCCTGC
GGGCCGCGCGCGAACACCGCCACGTCCTCGCCTGCGTGGGTCTCT
TCGTCCAGGGGCACTGCTGACTGCTGCCGATACTCGGGGCTCCCG
CTCTCGCTCTCGGTAACATCCGGCCGGGCGCCGTCCTTGAGCACA
TAGCCTGGACCGTTTCCGTATAGGAGGACCGTGTAGGCCTTCCTG
TCCCGGGCCTTGCCAGCGGCCAGCCCGATGAAGGAGCTCCCTCGC
AGGGGGTAGCCTCCGAAGGAGAAGACGTGGGAGTGGTCGGCAGTG
ACGAGGCTCAGCGTGTCCTCCTCGCTGGTGA.

In some embodiments, the AP gene fragment has the sequence of:

(SEQ ID NO: 44)
GCTGGCCCGCCCTCTCAATGGCGTCGTCGAACATGATCGTCTCAG
TCAGTGCCCGGTAAGCCCTGCTTTCATGATGACCATGGTCGATGC
GACCACCCTCCACGAAGAGGAAGAAGCCGCGGGGGTGTCTGCTCA
GCAGGCGCAGGGCAGCCTCTGTCATCTCCATCAGGGAGGGGTCCA
GTGTGGAGTCTCGGTGGATCTCGTATTTCATGTCTCCAGGCTCAA
AGAGACCCATGAGATGGGTCACAGACGGGTCCAGGGAAGCCTGCA
TGAGCTCAGTGCGGTTCCACACGTACCGGGCACCCTGGCGTTCGC
CGAGCCATTCCTGCACCAGATTCTTCCCGTCCAGCCTGGTCCCAC
CTTGGCTGTAGTCATCTGGGTACTCAGGGTCTGGGGTTCCCATGC
GAAACATGTACTTTCGGCCTCCA.

In some embodiments, the AP gene fragment has the sequence of:

(SEQ ID NO: 45)
CCCCGGGTGCGCGGCGTCGGTGGTGCCGGCGGGGGGCGCCAGGTC
GCAGGCGGTGTAGGGCTCCAGGCAGGCGGCGAAGGCCATGACGTG
CGCTATGAAGGTCTGCTCCTGCACGCCGTGAACCAGGTGCGCCTG
CGGGCCGCGCGCGAACACCGCCACGTCCTCGCCTGCGTGGGTCTC
TTCGTCCAGGGGCACTGCTGACTGCTGCCGATACTCGGGGCTCCC
GCTCTCGCTCTCGGTAACATCCGGCCGGGCGCCGTCCTTGAGCAC
ATAGCCTGGACCGTTTC

In some embodiments, the AP gene fragment has the sequence of:

(SEQ ID NO: 46)
CGTATAGGAGGACCGTGTAGGCCTTCCTGTCCCGGGCCTTGCCAG
CGGCCAGCCCGATGAAGGAGCTCCCTCGCAGGGGGTAGCCTCCGA
AGGAGAAGACGTGGGAGTGGTCGGCAGTGACGAGGCTCAGCGTGT
CCTCCTCGCTGGTGAGCTGGCCCGCCCTCTCAATGGCGTCGTCGA
ACATGATCGTCTCAGTCAGTGCCCGGTAAGCCCTGCTTTCATGAT
GACCATGGTCGATGCGACCACCCTCCACGAAGAGGAAGAAGCCGC
GGGGGTGTCTGCTCAGCAGG.

In some embodiments, the AP gene fragment has the sequence of:

(SEQ ID NO: 47)
CGCAGGGCAGCCTCTGTCATCTCCATCAGGGAGGGGTCCAGTGTG
GAGTCTCGGTGGATCTCGTATTTCATGTCTCCAGGCTCAAAGAGA
CCCATGAGATGGGTCACAGACGGGTCCAGGGAAGCCTGCATGAGC
TCAGTGCGGTTCCACACGTACCGGGCACCCTGGCGTTCGCCGAGC
CATTCCTGCACCAGATTCTTCCCGTCCAGCCTGGTCCCACCTTGG
CTGTAGTCATCTGGGTACTCAGGGTCTGGGGTTCCCATGCGAAAC
ATGTACTTTCGGCCTCCA.

Dual hybrid vectors for use in the methods and compositions described herein are designed such that approximately half of the stereocilin coding sequence is contained within each vector (e.g., each vector contains a polynucleotide that encodes approximately half of the stereocilin protein). The determination of how to split the polynucleotide sequence between the two nucleic acid vectors is made based on the size of the promoter and the locations of sequence elements of interest in the polynucleotide that encodes the stereocilin protein (e.g., exons of the STRC gene). The first vector in the dual hybrid vector system can contain a promoter sequence 5′ of a polynucleotide encoding an N-terminal portion of a stereocilin protein. The nucleic acid vectors can optionally contain STRC UTRs (e.g., full-length 5′ and/or 3′ UTRs) that are not part of the promoters described herein. One exemplary dual hybrid vector system includes a first nucleic acid vector containing a STRC promoter described hereinabove (e.g., a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide encoding an N-terminal portion of a stereocilin protein (e.g., human stereocilin, e.g., an N-terminal portion of SEQ ID NO: 3), a splice donor sequence 3′ of the polynucleotide sequence, and a recombinogenic region 3′ of the splice donor sequence; and a second nucleic acid vector containing a recombinogenic region (e.g., the same recombinogenic region that is included in the first vector), a splice acceptor sequence 3′ of the recombinogenic region, a polynucleotide encoding a C-terminal portion of the stereocilin protein (e.g., human stereocilin, e.g., a C-terminal portion of SEQ ID NO: 3) 3′ of the splice acceptor sequence, and a poly(A) sequence. In some embodiments, the STRC promoter is a polynucleotide having the sequence of SEQ ID NO: 1 or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the STRC promoter is a polynucleotide having the sequence of SEQ ID NO: 2 or a functional portion thereof, or a variant having at least 85% (e.g., at least 86%, 87%, 88%, 89%, 90%, 91%, 92%, 93%, 94%, 95%, 96%, 97%, 98%, 99%, or more) sequence identity to SEQ ID NO: 2 or a functional portion thereof. In some embodiments, the STRC promoter is a functional portion of SEQ ID NO: 2 that is capable of controlling expression of the STRC gene. In some embodiments, the functional portion of SEQ ID NO: 2 includes or has the sequence of nucleotides 252-537 of SEQ ID NO: 2. In some embodiments, the functional portion of SEQ ID NO: 2 includes or has the sequence of nucleotides 120-537 of SEQ ID NO: 2. In some embodiments, the functional portion of SEQ ID NO: 2 includes or has the sequence of nucleotides 35-530 of SEQ ID NO: 2. In some embodiments, the STRC promoter is a polynucleotide having the sequence of SEQ ID NO: 48 or a functional portion thereof, or a variant having at least 85% (e.g., at least 86%, 87%, 88%, 89%, 90%, 91%, 92%, 93%, 94%, 95%, 96%, 97%, 98%, 99%, or more) sequence identity SEQ ID NO: 48 or a functional portion thereof. In some embodiments, the STRC promoter is a functional portion of SEQ ID NO: 48 that is capable of controlling expression of the STRC gene. In some embodiments, the functional portion of SEQ ID NO: 48 includes or has the sequence of nucleotides 280-560 of SEQ ID NO: 48. In some embodiments, the functional portion of SEQ ID NO: 48 includes or has the sequence of nucleotides 280-564 of SEQ ID NO: 48. In some embodiments, the functional portion of SEQ ID NO: 48 includes or has the sequence of nucleotides 124-560 of SEQ ID NO: 48. In some embodiments, the functional portion of SEQ ID NO: 48 includes or has the sequence of nucleotides 124-564 of SEQ ID NO: 48. In some embodiments, the functional portion of SEQ ID NO: 48 includes or has the sequence of nucleotides 1-560 of SEQ ID NO: 48. The first and second nucleic acid vectors can also contain the full length 5′ and/or 3′ STRC UTRs, respectively (e.g., the human STRC 5′ UTR can be included in the first nucleic acid vector, and the human STRC 3′ UTR can be included in the second nucleic acid vector).

Another exemplary dual hybrid vector system that includes a STRC promoter includes a first nucleic acid vector containing a STRC promoter described hereinabove (e.g., a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to polynucleotide encoding an N-terminal portion of a stereocilin protein (e.g., murine stereocilin, e.g., an N-terminal portion of SEQ ID NO: 4), a splice donor sequence 3′ of the polynucleotide sequence, and a recombinogenic region 3′ of the splice donor sequence; and a second nucleic acid vector containing a recombinogenic region (e.g., the same recombinogenic region that is included in the first vector), a splice acceptor sequence 3′ of the recombinogenic region, a polynucleotide encoding a C-terminal portion of the stereocilin protein (e.g., murine stereocilin, e.g., a C-terminal portion of SEQ ID NO: 4) 3′ of the splice acceptor sequence, and a poly(A) sequence. The first and second nucleic acid vectors can also contain the full length 5′ and/or 3′ STRC UTRs, respectively (e.g., the mouse STRC 5′ UTR can be included in the first nucleic acid vector, and the mouse STRC 3′ UTR can be included in the second nucleic acid vector). To accommodate a STRC UTR, the stereocilin coding sequence can be divided at a different position than it would be in a dual hybrid vector system that does not include a STRC UTR.

In some embodiments, the stereocilin protein has the sequence of SEQ ID NO: 3 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the stereocilin protein has the sequence of SEQ ID NO: 4 or at least 85% (e.g., at least 86%, 87%, 88%, 89%, 90%, 91%, 92%, 93%, 94%, 95%, 96%, 97%, 98%, 99%, or more) sequence identity thereto. In some embodiments, the polynucleotide that encodes a full-length human stereocilin protein has the sequence of SEQ ID NO: 5 or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 5. In some embodiments, the polynucleotide having at least 85% sequence identity to SEQ ID NO: 5 encodes the stereocilin protein of SEQ ID NO: 3. In some embodiments, the polynucleotide that encodes a full-length murine stereocilin protein has the sequence of SEQ ID NO: 6 or is a variant thereof having at least 85% (e.g., at least 86%, 87%, 88%, 89%, 90%, 91%, 92%, 93%, 94%, 95%, 96%, 97%, 98%, 99%, or more) sequence identity SEQ ID NO: 6. In some embodiments, the polynucleotide having at least 85% sequence identity to SEQ ID NO: 6 encodes the stereocilin protein of SEQ ID NO: 4.

In some embodiments, the first vector in a dual hybrid vector system for use in the compositions and methods described herein contains nucleotides 1-1800 of a polynucleotide encoding murine stereocilin (e.g., nucleotides 1-1800 of SEQ ID NO: 6), and the second vector contains the remaining nucleotides of the polynucleotide encoding murine stereocilin (e.g., nucleotides 1801-5430 of SEQ ID NO: 6). In some embodiments, the polynucleotide encoding murine stereocilin is divided between the first and second vectors at a STRC exon boundary. In some embodiments, the exon boundary is the exon 4/exon 5 boundary, the exon 5/exon6 boundary, the exon 6/exon 7 boundary, or the exon 7/exon 8 boundary. In a dual hybrid vector system in which the murine STRC coding sequence is divided at the exon 4/exon 5 boundary, the first vector contains nucleotides 1-2247 of a polynucleotide encoding stereocilin (e.g., nucleotides 1-2247 of SEQ ID NO: 6), and the second vector contains the remaining nucleotides of the polynucleotide encoding stereocilin (e.g., nucleotides 2248-5430 of SEQ ID NO: 6). In a dual hybrid vector system in which the murine STRC coding sequence is divided at the exon 5/exon 6 boundary, the first vector contains nucleotides 1-2310 of a polynucleotide encoding stereocilin (e.g., nucleotides 1-2310 of SEQ ID NO: 6), and the second vector contains the remaining nucleotides of the polynucleotide encoding stereocilin (e.g., nucleotides 2311-5430 of SEQ ID NO: 6). In a dual hybrid vector system in which the murine STRC coding sequence is divided at the exon 6/exon 7 boundary, the first vector contains nucleotides 1-2421 of a polynucleotide encoding stereocilin (e.g., nucleotides 1-2421 of SEQ ID NO: 6), and the second vector contains the remaining nucleotides of the polynucleotide encoding stereocilin (e.g., nucleotides 2422-5430 of SEQ ID NO: 6). In a dual hybrid vector system in which the murine STRC coding sequence is divided at the exon 7/exon 8 boundary, the first vector contains nucleotides 1-2588 of a polynucleotide encoding stereocilin (e.g., nucleotides 1-2588 of SEQ ID NO: 6), and the second vector contains the remaining nucleotides of the polynucleotide encoding stereocilin (e.g., nucleotides 2589-5430 of SEQ ID NO: 6).

The dual hybrid vectors used in the methods and compositions described herein can optionally include a degradation signal sequence in both the first and second nucleic acid vectors. The degradation signal sequence can be included to prevent or reduce the expression of portions of the stereocilin protein from polynucleotides that failed to recombine and/or undergo splicing. The degradation signal sequence is positioned 3′ of the recombinogenic region in the first nucleic acid vector and is positioned between the recombinogenic region and the splice acceptor in the second nucleic acid vector. Suitable degradation signal sequences that can be used in the compositions and methods described herein are known in the art and are described, for example, in International Application Publication No. WO 2016/139321, which is incorporated herein by reference.

In some embodiments, the first member of the dual vector system includes the STRC promoter of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 280-560 of SEQ ID NO: 48 or a variant thereof having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 280-560 of SEQ ID NO: 48 operably linked to a polynucleotide that encodes an N-terminal portion of a stereocilin protein. The polynucleotide sequences that encode an N-terminal portion of a stereocilin protein can be partially or fully codon-optimized for expression. In some embodiments, the first member of the dual vector system includes a splice donor sequence. In some embodiments, the first member of the dual vector system includes an AP gene fragment described herein (e.g., any one of SEQ ID NOs: 42-47, such as SEQ ID NO: 45). In some embodiments, the first member of the dual vector system is flanked on each of the 5′ and 3′ sides by an ITR. In some embodiments, the flanking ITRs are any variant of AAV2 inverted terminal repeats that can be encapsidated by a plasmid that carries the AAV2 Rep gene. It will be understood by those of skill in the art that, for any given pair of ITR sequences in a transfer plasmid that is used to create the viral vector (typically by transfecting cells with that plasmid together with other plasmids carrying the necessary AAV genes for viral vector formation), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom.

In some embodiments, the second member of the dual vector system includes a polynucleotide that encodes the C-terminal portion of the stereocilin protein immediately followed by a stop codon. The polynucleotide sequences that encode the C-terminal portion of the stereocilin protein can be partially or fully codon-optimized for expression. In some embodiments, the second member of the dual vector system includes a splice acceptor sequence. In some embodiments, the second member of the dual vector system includes an AP gene fragment described herein (e.g., any one of SEQ ID NOs: 42-47, such as SEQ ID NO: 45). In some embodiments, the second member of the dual vector system includes a poly(A) sequence. In particular embodiments, the second member of the dual vector system is flanked on each of the 5′ and 3′ sides by an ITR. In some embodiments, the flanking ITRs are any variant of AAV2 ITRs that can be encapsidated by a plasmid that carries the AAV2 Rep gene. It will be understood by those of skill in the art that, for any given pair of inverted terminal repeat sequences in a transfer plasmid that is used to create the viral vector (typically by transfecting cells with that plasmid together with other plasmids carrying the necessary AAV genes for viral vector formation), that the corresponding sequence in the viral vector can be altered due to the ITRs adopting a “flip” or “flop” orientation during recombination. Thus, the sequence of the ITR in the transfer plasmid is not necessarily the same sequence that is found in the viral vector prepared therefrom.

A transfer plasmid (e.g., a plasmid containing a DNA sequence to be delivered by a nucleic acid vector, e.g., to be delivered by an AAV) may be co-delivered into producer cells with a helper plasmid (e.g., a plasmid providing proteins necessary for AAV manufacture) and a rep/cap plasmid (e.g., a plasmid that provides AAV capsid proteins and proteins that insert the transfer plasmid DNA sequence into the capsid shell) to produce a nucleic acid vector (e.g., an AAV vector) for administration. Nucleic acid vectors (e.g., a nucleic acid vector (e.g., an AAV vector) containing a polynucleotide encoding an N-terminal portion of a stereocilin protein and a nucleic acid vector (e.g., an AAV vector) containing a polynucleotide encoding a C-terminal portion a stereocilin protein) can be combined (e.g., in a single formulation) prior to administration.

Other exemplary pairs of overlapping, trans-splicing, and dual hybrid vectors are described in Table 4 below.

TABLE 4
Representative pairs of overlapping, trans-splicing, and hybrid dual
vectors for use in the methods and compositions described herein

| Vector | | |
|---|---|---|
| Pair | | |
| Number | Vector Type | Vector Pair |
| 1 | Overlapping | First nucleic acid vector contains: a STRC promoter (e.g., SEQ ID NO: 1, |
| | | SEQ ID NO: 2, SEQ ID NO: 48, a functional portion of SEQ ID NO: 2 that |
| | | includes nucleotides 252-537 or 35-530 of SEQ ID NO: 2, or a functional |
| | | portion of SEQ ID NO: 48 containing nucleotides 280-560 of SEQ ID NO: |
| | | 48 or a variant thereof having at least 85% (e.g., at least 86%, 87%, 88%, |
| | | 89%, 90%, 91%, 92%, 93%, 94%, 95%, 96%, 97%, 98%, 99%, or more) |
| | | sequence identity to the nucleic acid sequence of SEQ ID NO: 1, SEQ ID |
| | | NO: 2, SEQ ID NO: 48, a functional portion of SEQ ID NO: 2 that includes |
| | | nucleotides 252-537 or 35-530 of SEQ ID NO: 2, or a functional portion of |
| | | SEQ ID NO: 48 containing nucleotides 280-560 of SEQ ID NO: 48) |
| | | operably linked to a polynucleotide encoding an N-terminal portion of a |
| | | human stereocilin protein (e.g., an N-terminal portion of SEQ ID NO: 3), in |
| | | which the polynucleotide encoding the N-terminal portion of the human |
| | | stereocilin protein includes the 500 bp 3′ of the position selected as the |
| | | central point of the overlapping region of STRC |
| | | Second nucleic acid vector contains: a polynucleotide encoding a C- |
| | | terminal portion of the human stereocilin protein (e.g., a C-terminal portion |
| | | of SEQ ID NO: 3) and a poly(A) sequence, in which the polynucleotide |
| | | encoding the C-terminal portion of the human stereocilin protein includes |
| | | the 500 bp 5′ of the position selected as the central point of the |
| | | overlapping region of STRC |
| 2 | Trans- | First nucleic acid vector contains: a STRC promoter (e.g., SEQ ID NO: 1, |
| | splicing | SEQ ID NO: 2, SEQ ID NO: 48, a functional portion of SEQ ID NO: 2 that |
| | | includes nucleotides 252-537 or 35-530 of SEQ ID NO: 2, or a functional |
| | | portion of SEQ ID NO: 48 containing nucleotides 280-560 of SEQ ID NO: |
| | | 48 or a variant thereof having at least 85% (e.g., at least 86%, 87%, 88%, |
| | | 89%, 90%, 91%, 92%, 93%, 94%, 95%, 96%, 97%, 98%, 99%, or more) |
| | | sequence identity to the nucleic acid sequence of SEQ ID NO: 1, SEQ ID |
| | | NO: 2, SEQ ID NO: 48, a functional portion of SEQ ID NO: 2 that includes |
| | | nucleotides 252-537 or 35-530 of SEQ ID NO: 2, or a functional portion of |
| | | SEQ ID NO: 48 containing nucleotides 280-560 of SEQ ID NO: 48) |
| | | operably linked to a polynucleotide encoding an N-terminal portion of a |
| | | human stereocilin protein (e.g., an N-terminal portion of SEQ ID NO: 3) |
| | | and a splice donor sequence 3′ of the polynucleotide |
| | | Second nucleic acid vector contains: a splice acceptor sequence 5′ of a |
| | | polynucleotide encoding a C-terminal portion of the human stereocilin |
| | | protein (e.g., a C-terminal portion of SEQ ID NO: 3) and a poly(A) |
| | | sequence |
| 3 | Hybrid | First nucleic acid vector contains: a STRC promoter (e.g., SEQ ID NO: |
| | | 1, SEQ ID NO: 2, SEQ ID NO: 48, a functional portion of SEQ ID NO: 2 |
| | | that includes nucleotides 252-537 or 35-530 of SEQ ID NO: 2, or a |
| | | functional portion of SEQ ID NO: 48 containing nucleotides 280-560 of |
| | | SEQ ID NO: 48 or a variant thereof having at least 85% (e.g., at least |
| | | 86%, 87%, 88%, 89%, 90%, 91%, 92%, 93%, 94%, 95%, 96%, 97%, 98%, |
| | | 99%, or more) sequence identity to the nucleic acid sequence of SEQ ID |
| | | NO: 1, SEQ ID NO: 2, SEQ ID NO: 48, a functional portion of SEQ ID NO: |
| | | 2 that includes nucleotides 252-537 or 35-530 of SEQ ID NO: 2, or a |
| | | functional portion of SEQ ID NO: 48 containing nucleotides 280-560 of |
| | | SEQ ID NO: 48) operably linked to a polynucleotide encoding an |
| | | N-terminal portion of a human stereocilin protein (e.g., an N-terminal portion |
| | | of SEQ ID NO: 3), a splice donor sequence 3′ of the polynucleotide, and a |
| | | recombinogenic region 3′ of the splice donor sequence |
| | | Second nucleic acid vector contains: a recombinogenic region, a splice |
| | | acceptor sequence 3′ of the recombinogenic region, a polynucleotide |
| | | encoding a C-terminal portion of the human stereocilin protein (e.g., a C- |
| | | terminal portion of SEQ ID NO: 3) 3′ of the splice acceptor sequence, and |
| | | a poly(A) sequence |

### Intein Expression Systems

Another gene therapy approach for expressing large proteins in mammalian cells involves the use of inteins. An intein, also known as a “protein intron,” is a portion of a protein that is typically 100-900 amino acid residues long and is capable of self-excision and ligation of the N- and C-terminal residues of the flanking protein fragments (“exteins”). Inteins can be divided into three different classes, including maxi-intein, mini-intein, and split intein. Maxi-inteins refer to N- and C-terminal splicing regions of a protein interrupted by a homing endonuclease domain (HEG). HEGs refer to a class of endonucleases encoded as stand-alone genes within introns, as protein fusions with other proteins, or as self-splicing inteins. HEGs generally hydrolyze very few and select DNA regions. Once a HEG hydrolyzes a piece of DNA, the gene encoding the HEG typically incorporates itself into the cleavage site, thereby increasing its allele frequency. “Mini-inteins” refer to N- and C-terminal splicing domains lacking the HEG domain. “Split inteins” refer to inteins that are transcribed and translated as two separate polypeptides that are joined with an extein. Alanine inteins are another class of inteins that have a splicing junction of an alanine instead of a cysteine or serine.

The splicing domain of inteins contains two subdomains, namely the N- and C-terminal splicing domains, which contain conserved motifs with conserved residues that mediate the splicing activity. The N-terminal splicing domain contains A, N2, B, and N4 structural motifs, whereas the C-terminal splicing domain contains F and G motifs. The A-motif contains Cys/Ser or Thr as conserved residues; the B motif includes His and Thr residues; F motif contains Asp and His residues; G motifs carry two conserved residues, which include a penultimate His and a terminal Asn. C, D, E, and H motifs are generally related to the HEG domain in maxi-inteins.

Intein splicing falls within three distinct strategies: 1) class 1 (or classical/canonical) intein splicing which involves (a) a (N—S/N—O) acyl shift that transforms the peptide bond of an N-terminal splice junction to a thio(ester) linkage, (b) transesterification reaction that forms a branched intermediate, (c) Asn cyclization, which removes the branched intermediate by cleaving the C-terminal splice junction, and (d) a second (S—N/O—N) acyl shift that ligates the flanking extein segments through amide bond formation; 2) class 2 inteins (also known as Alanine-inteins) bypass step (a) of the classical splicing reaction; and 3) class 3 mechanism which involves the formation of two branched intermediates.

Among the various intein systems described above, the split intein trans-splicing approach has been demonstrated to successfully overcome the size limitations of traditional gene therapy vectors (e.g., AAV: approximately 4.7 kb maximal size limit). For example, Subramanyam et al. (PNAS 110:15461-6 (2013)) have employed the split intein system to reconstitute the α1C-subunit of L-type calcium channel in cardiomyocytes from two separate halves. Similarly, Truong et al. (Nucleic Acids Res. 43:6450-8 (2015)) have shown successful reconstitution of two halves of the Cas9 protein using a split intein system. Accordingly, the present disclosure provides split intein trans-splicing systems for the packaging and delivery of a stereocilin coding sequence that is operably linked to a STRC promoter. This method allows for two separate polynucleotides, each containing approximately one half of the STRC gene and including a polynucleotide sequence encoding an N-intein fragment or a C-intein fragment, to be expressed from two separate expression vectors (e.g., any one of the nucleic acid vectors disclosed herein) and post-translationally reconstituted to produce a full-length stereocilin protein. Such systems may be incorporated into nucleic acid expression vectors disclosed herein, such as, e.g., rAAV vectors.

In one example, the present disclosure provides a two-vector split intein system containing: a) a first nucleic acid vector containing a polynucleotide that includes a sequence encoding an N-terminal portion of a human stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3), in which the sequence encoding an N-terminal portion of a human stereocilin protein includes at its 3′ end an in-frame polynucleotide sequence encoding an N-intein; and b) a second vector containing a polynucleotide that includes a sequence encoding a C-terminal portion of a human stereocilin protein (e.g., a C-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3), in which the sequence encoding a C-terminal portion of a human stereocilin protein includes at its 5′ end an in-frame polynucleotide sequence encoding a C-intein.

In another example, the present disclosure provides a two-vector split intein system containing: a) a first vector containing a polynucleotide that includes a sequence encoding an N-terminal portion of a murine stereocilin protein (e.g., an N-terminal portion of SEQ ID NO: 4 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 4), in which the sequence encoding an N-terminal portion of a murine stereocilin protein includes at its 3′ end an in-frame polynucleotide sequence encoding an N-intein; and b) a second vector containing a polynucleotide that includes a sequence encoding a C-terminal portion of a murine stereocilin protein (e.g., a C-terminal portion of SEQ ID NO: 4 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 4), in which the sequence encoding a C-terminal portion of a murine stereocilin protein includes at its 5′ end an in-frame nucleic acid sequence encoding a C-intein.

In some embodiments, the sequence encoding an N-terminal portion of a murine stereocilin protein is a sequence encoding amino acids 1-730 of the stereocilin protein (e.g., amino acids 1-730 of SEQ ID NO: 4) and the sequence encoding the C-terminal portion of the murine stereocilin protein encodes the remaining amino acids of the stereocilin protein (e.g., amino acids 731-1809 of SEQ ID NO: 4). In this embodiment, the sequence of the N-terminal portion is nucleotides 1-2190 of the polynucleotide encoding murine stereocilin (e.g., nucleotides 1-2190 SEQ ID NO: 6) and the sequence of the C-terminal portion is from nucleotide 2191 to the 3′ end of the coding sequence of the polynucleotide encoding murine stereocilin (e.g., the remaining nucleotides of SEQ ID NO: 6, e.g., nucleotides 2191-5430). In some embodiments, the sequence encoding an N-terminal portion of a murine stereocilin protein is a sequence encoding amino acids 1-746 of the stereocilin protein (e.g., amino acids 1-746 of SEQ ID NO: 4) and the sequence encoding the C-terminal portion of the murine stereocilin protein encodes the remaining amino acids of the stereocilin protein (e.g., amino acids 747-1809 of SEQ ID NO: 4). In this embodiment, the sequence of the N-terminal portion is nucleotides 1-2238 of the polynucleotide encoding murine stereocilin (e.g., nucleotides 1-2238 SEQ ID NO: 6) and the sequence of the C-terminal portion is from nucleotide 2239 to the 3′ end of the coding sequence of the polynucleotide encoding murine stereocilin (e.g., the remaining nucleotides of SEQ ID NO: 6, e.g., nucleotides 2239-5430). In some embodiments, the sequence encoding an N-terminal portion of a murine stereocilin protein is a sequence encoding amino acids 1-969 of the stereocilin protein (e.g., amino acids 1-969 of SEQ ID NO: 4) and the sequence encoding the C-terminal portion of the murine stereocilin protein encodes the remaining amino acids of the stereocilin protein (e.g., amino acids 970-1809 of SEQ ID NO: 4). In this embodiment, the sequence of the N-terminal portion is nucleotides 1-2907 of the polynucleotide encoding murine stereocilin (e.g., nucleotides 1-2907 SEQ ID NO: 6) and the sequence of the C-terminal portion is from nucleotide 2908 to the 3′ end of the coding sequence of the polynucleotide encoding murine stereocilin (e.g., the remaining nucleotides of SEQ ID NO: 6, e.g., nucleotides 2908-5430). In some embodiments, the sequence encoding an N-terminal portion of a murine stereocilin protein is a sequence encoding amino acids 1-1002 of the stereocilin protein (e.g., amino acids 1-1002 of SEQ ID NO: 4) and the sequence encoding the C-terminal portion of the murine stereocilin protein encodes the remaining amino acids of the stereocilin protein (e.g., amino acids 1003-1809 of SEQ ID NO: 4). In this embodiment, the sequence of the N-terminal portion is nucleotides 1-3006 of the polynucleotide encoding murine stereocilin (e.g., nucleotides 1-3006 SEQ ID NO: 6) and the sequence of the C-terminal portion is from nucleotide 3007 to the 3′ end of the coding sequence of the polynucleotide encoding murine stereocilin (e.g., the remaining nucleotides of SEQ ID NO: 6, e.g., nucleotides 3007-5430). Since split inteins require the presence of extein amino acid residues that are not excised from the target protein during full-length protein recombination, the above split sites were selected based on the identification of a partial extein sequence in the peptide sequence of the stereocilin protein. For certain split sites (e.g., the split sites at amino acids 730 and 1002), the full extein sequence can be produced by incorporating a sequence encoding a short peptide at the split site so the short peptide will be fused to the STRC protein at the split site, thereby providing the catalytic amino acid residue.

In some embodiments, both the first vector and the second vector further include a promoter sequence, such as a STRC promoter sequence (e.g., a STRC promoter sequence of any one of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 280-560 of SEQ ID NO: 48 or a variant thereof having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 280-560 of SEQ ID NO: 48) operably linked to the 5′ end of the polynucleotide encoding the first fusion protein (an N-terminal portion of a stereocilin protein fused to an N-intein) and/or to the 5′ end of the polynucleotide encoding the second fusion protein (a C-terminal portion of a stereocilin protein fused to a C-intein). In some embodiments, the STRC promoter has the sequence of SEQ ID NO: 1 or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 1. In some embodiments, the STRC promoter has the sequence of SEQ ID NO: 2 or a portion thereof or is a variant thereof having at least 85% (e.g., at least 86%, 87%, 88%, 89%, 90%, 91%, 92%, 93%, 94%, 95%, 96%, 97%, 98%, 99%, or more) sequence identity to SEQ ID NO: 2 or a portion thereof. In some embodiments, the STRC promoter is a functional portion of SEQ ID NO: 2 that is capable of controlling expression of the STRC gene. In some embodiments, the functional portion of SEQ ID NO: 2 includes or has the sequence of nucleotides 252-537 of SEQ ID NO: 2. In some embodiments, the functional portion of SEQ ID NO: 2 includes or has the sequence of nucleotides 120-537 of SEQ ID NO: 2. In some embodiments, the functional portion of SEQ ID NO: 2 includes or has the sequence of nucleotides 35-530 of SEQ ID NO: 2. In some embodiments, the STRC promoter is a polynucleotide having the sequence of SEQ ID NO: 48 or a functional portion thereof, or a variant having at least 85% (e.g., at least 86%, 87%, 88%, 89%, 90%, 91%, 92%, 93%, 94%, 95%, 96%, 97%, 98%, 99%, or more) sequence identity SEQ ID NO: 48 or a functional portion thereof. In some embodiments, the STRC promoter is a functional portion of SEQ ID NO: 48 that is capable of controlling expression of the STRC gene. In some embodiments, the functional portion of SEQ ID NO: 48 includes or has the sequence of nucleotides 280-560 of SEQ ID NO: 48. In some embodiments, the functional portion of SEQ ID NO: 48 includes or has the sequence of nucleotides 280-564 of SEQ ID NO: 48. In some embodiments, the functional portion of SEQ ID NO: 48 includes or has the sequence of nucleotides 124-560 of SEQ ID NO: 48. In some embodiments, the functional portion of SEQ ID NO: 48 includes or has the sequence of nucleotides 124-564 of SEQ ID NO: 48. In some embodiments, the functional portion of SEQ ID NO: 48 includes or has the sequence of nucleotides 1-560 of SEQ ID NO: 48. In some embodiments, the STRC promoter in the second nucleic acid vector is the same (i.e., has the same nucleotide sequence) as the STRC promoter in the first nucleic acid vector. In some embodiments, the STRC promoter in the second nucleic acid vector has a different nucleotide sequence than the STRC promoter in the first nucleic acid vector.

In some embodiments, the N-intein and the C-intein are derived from the same intein or split intein gene. Alternatively, the N-intein and the C-intein sequences derive from two different intein genes that can perform protein trans-splicing to reconstitute a full-length stereocilin protein. In some embodiments, the same gene is from the same organism or from different organisms. Commonly used split inteins derive from the DnaEgene from various organisms. In some embodiments, the polynucleotide encoding a stereocilin protein is split into two portions, each corresponding to approximately half of the total coding sequence of the full-length gene, namely a N-terminal portion and a C-terminal portion. The polynucleotide encoding the N-terminal portion of stereocilin is fused in frame at its 3′ end with the polynucleotide encoding the N-intein, whereas the polynucleotide encoding the C-terminal portion of stereocilin is fused in frame at its 5′ end with the polynucleotide encoding the C-intein.

In some embodiments, the first vector and the second vector, when introduced into a cell (e.g., a cell of a subject, such as a subject with sensorineural hearing loss, e.g., DFNB16) produce a first fusion protein and a second fusion protein. In some embodiments, the first fusion protein contains the N-terminal portion of the stereocilin protein fused at its C-terminus with the N-intein. In some embodiments, the second fusion protein contains the C-terminal portion of the stereocilin protein fused at its N-terminus with the C-intein. In some embodiments, the N-intein of the first fusion protein and the C-intein of the second fusion protein selectively bind to produce a third fusion protein containing from N-terminus to C-terminus: an N-terminal portion of the stereocilin protein, an N-intein bound at its C-terminus to the C-intein, and the C-terminal portion of the stereocilin protein. In some embodiments, the N-intein bound to the C-intein is capable of performing a trans-splicing reaction that excises the N-intein and the C-intein and ligates the C-terminus of the N-terminal portion and the N-terminus of the C-terminal portion of the stereocilin protein.

The split intein system described herein may include split inteins that are encoded by one gene that is subsequently engineered using routine methods to encode two separate intein fragments (e.g., a split intein). In some embodiments, the split inteins are encoded by two separate genes.

Split inteins of the disclosed compositions and methods may be derived from the DnaEgene (e.g., DNA polymerase III subunit alpha) from cyanobacteria, such as, e.g., Nostoc punctiforme (Npu), Synechocystis sp. PCC6803 (Ssp), Fischerella sp. PCC9605 (Fsp), Scytonema tolypothrichoides (Sto), Cyanobacteria bacterium SW_9_47_5, Nodularia spumigena (Nsp), Nostoc flagelliforme (Nfl), Crocosphaera watsonii (Cwa) WH8502, Chroococcidiopsis cubana (Ccu) CCALA043, Trichodesmium erythraeum (Ter), Rhodothermus marinus (Rma), Saccharomyces cerevisiae (Sce), Saccharomyces castellii (Sca), Saccharomyces unisporus (Sun), Zygosaccharomyces bisporus (Zbi), Torulaspora pretoriensis (Tpr), Mycobacteria tuberculosis (Mtu), Mycobacterium leprae (Mle), Mycobacterium smegmatis (Msm), Pyrococcus abyssi (Pab), Pyrococcus horikoshii (Pho), Coxiella burnetti (Cbu), Coxiella neoformans (Cne), Coxiella gattii (Cga), Histoplasma capsulatum (Hca), and Porphyra purpurea chloroplast (Ppu), among others. In some embodiments, the split intein is derived from multiple sequence alignment studies of DnaE for identifying a consensus design (e.g., Cfa) to engineer a split intein with desirable stability and activity (e.g., the split inteins are Cfa inteins). Other split intein systems suitable for use with the presently disclosed compositions and methods include those described in International Patent Application Publication Nos. WO 2020/249723, WO 2021/099607, and WO 2021/047558, US Patent Application Publication Nos. US20210371878A1, US20220275027A1, and US20200277333A1, and U.S. Pat. Nos. 10,066,027, 10,526,401, 11,142,550, 11,306,324, 10,100,080, and 8,394,604, each of which is incorporated herein by reference herein as it relates to split intein systems.

In some embodiments, each of the first vector and the second vector further include a 5′ ITR at its 5′ end and a 3′ ITR and its 3′ end. In some embodiments, the 5′ ITR and the 3′ ITR are AAV ITRs. In some embodiments, the AAV ITRs are AAV2 ITRs.

In some embodiments, the two-vector split intein system of the disclosure includes: a) a first vector containing from 5′ to 3′: i) optionally, a 5′ ITR (e.g., AAV2 5′ ITR); ii) a polynucleotide containing a STRC promoter (e.g., a STRC promoter of any one of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 280-560 of SEQ ID NO: 48 or a variant thereof having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 289-560 of SEQ ID NO: 48); iii) a polynucleotide encoding an N-terminal portion of a stereocilin protein (e.g., an N-terminal portion of the stereocilin protein of SEQ ID NO: 3 or SEQ ID NO: 4); iv) a polynucleotide encoding an N-intein; (v) optionally, a poly(A) sequence; and (vi) optionally, a 3′ ITR (e.g., AAV2 3′ ITR); and b) a second vector containing from 5′ to 3′: i) optionally, a 5′ ITR (e.g., AAV2 5′ ITR); ii) a polynucleotide containing a STRC promoter (e.g., a STRC promoter of any one of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 280-560 of SEQ ID NO: 48, or a variant thereof having at least 85% (e.g., at least 86%, 87%, 88%, 89%, 90%, 91%, 92%, 93%, 94%, 95%, 96%, 97%, 98%, 99%, or more) sequence identity to the nucleic acid sequence of SEQ ID NO: 1, SEQ ID NO: 2, SEQ ID NO: 48, a functional portion of SEQ ID NO: 2 that includes nucleotides 252-537 or 35-530 of SEQ ID NO: 2, or a functional portion of SEQ ID NO: 48 containing nucleotides 280-560 of SEQ ID NO: 48); iii) a polynucleotide encoding a C-intein; iv) a polynucleotide encoding a C-terminal portion of the stereocilin protein (e.g., a C-terminal portion of the stereocilin protein of SEQ ID NO: 3 or SEQ ID NO: 4); (v) optionally, a poly(A) sequence; and (vi) optionally, a 3′ ITR (e.g., AAV2 3′ ITR).

In some embodiments, the two-vector split intein system of the disclosure includes a polynucleotide encoding an N-intein peptide having an amino acid sequence of SEQ ID NO: 7 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 7, as is shown below.

(SEQ ID NO: 7)
CLSYDTEILTVEYGFLPIGKIVEERIECTVYTVDKNGFVYTQPIA
QWHNRGEQEVFEYCLEDGSIIRATKDHKFMTTDGQMLPIDEIFER
GL

In some embodiments, the two-vector split intein system of the disclosure includes a polynucleotide encoding a C-intein peptide having an amino acid sequence of SEQ ID NO: 8 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 8, as is shown below.

(SEQ ID NO: 8)
VKIISRKSLGTQNVYDIGVEKDHNFLLKNGLVASN

In some embodiments, the two-vector split intein system of the disclosure includes a polynucleotide encoding an N-intein peptide having an amino acid sequence of SEQ ID NO: 9 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 9, as is shown below.

(SEQ ID NO: 9)
CLSYDTEILTVEYGFLPIGKIVEERIECTVYTVDKNGFVYTQPIA
QWHNRGEQEVFEYCLEDGSIIRATKDHKFMTTDGQMLPIDEIFER
GLDLKQVDGLP

In some embodiments, the two-vector split intein system of the disclosure includes a polynucleotide encoding a C-intein peptide having an amino acid sequence of SEQ ID NO: 10 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 10, as is shown below.

(SEQ ID NO: 10)
MVKIISRKSLGTQNVYDIGVEKDHNFLLKNGLVASN

In some embodiments, the two-vector split intein system of the disclosure includes a polynucleotide encoding a C-intein peptide having an amino acid sequence of SEQ ID NO: 11 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 11, as is shown below.

(SEQ ID NO: 11)
VKIISRKSLGTQNVYDIGVGEPHNFLLKNGLVASN

In some embodiments, the two-vector split intein system includes a first vector including a polynucleotide encoding an N-intein peptide having an amino acid sequence of SEQ ID NO: 7 or SEQ ID NO: 9 (e.g., positioned 3′ of a polynucleotide encoding an N-terminal portion of a stereocilin protein) and a second vector including a polynucleotide encoding a C-intein polypeptide having an amino acid sequence of SEQ ID NO: 8, SEQ ID NO: 10, or SEQ ID NO: 11 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 7 and a second vector including a polynucleotide encoding a C-intein polypeptide having an amino acid sequence of SEQ ID NO: 8. In some embodiments, the two-vector split intein system includes a first vector including a polynucleotide encoding an N-intein peptide having an amino acid sequence of SEQ ID NO: 7 and a second vector including a polynucleotide encoding a C-intein polypeptide having an amino acid sequence of SEQ ID NO: 10. In some embodiments, the two-vector split intein system includes a first vector including a polynucleotide encoding an N-intein peptide having an amino acid sequence of SEQ ID NO: 7 and a second vector including a polynucleotide encoding a C-intein polypeptide having an amino acid sequence of SEQ ID NO: 11. In some embodiments, the two-vector split intein system includes a first vector including a polynucleotide encoding an N-intein peptide having an amino acid sequence of SEQ ID NO: 9 and a second vector including a polynucleotide encoding a C-intein polypeptide having an amino acid sequence of SEQ ID NO: 8. In some embodiments, the two-vector split intein system includes a first vector including a polynucleotide encoding an N-intein peptide having an amino acid sequence of SEQ ID NO: 9 and a second vector including a polynucleotide encoding a C-intein polypeptide having an amino acid sequence of SEQ ID NO: 10. In some embodiments, the two-vector split intein system includes a first vector including a polynucleotide encoding an N-intein peptide having an amino acid sequence of SEQ ID NO: 9 and a second vector including a polynucleotide encoding a C-intein polypeptide having an amino acid sequence of SEQ ID NO: 11.

In some embodiments, the two-vector split intein system of the disclosure includes a polynucleotide encoding an N-intein peptide having an amino acid sequence of SEQ ID NO: 12 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 12, as is shown below.

(SEQ ID NO: 12)
CLSYETEILTVEYGLLPIGKIVEKRIECTVYSVDNNGNIYTQPVA
QWHDRGEQEVFEYCLEDGSLIRATKDHKFMTVDGQMLPIDEIFER
ELDLMRVDNLPN

In some embodiments, the two-vector split intein system of the disclosure includes a polynucleotide encoding a C-intein peptide having an amino acid sequence of SEQ ID NO: 13 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 13, as is shown below.

(SEQ ID NO: 13)
MIKIATRKYLGKQNVYDIGVERDHNFALKNGFIASN

In some embodiments, the two-vector split intein system includes a first vector including a polynucleotide encoding an N-intein peptide having an amino acid sequence of SEQ ID NO: 12 (e.g., positioned 3′ of a polynucleotide encoding an N-terminal portion of a stereocilin protein) and a second vector including a polynucleotide encoding a C-intein polypeptide having an amino acid sequence of SEQ ID NO: 13 (e.g., positioned 5′ of a polynucleotide encoding a C-terminal portion of a stereocilin protein).

In some embodiments, the two-vector split intein system of the disclosure includes a polynucleotide encoding an N-intein peptide having an amino acid sequence of SEQ ID NO: 14 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 14, as is shown below.

(SEQ ID NO: 14)
CLSYDTEILTVEYGFLPIGKIVEERIECTVYTVDKNGFVYTQPIA
QWHNRGEQEVFEYCLEDGSIIRATKDHKFMTTDGQMLPIDEIFER
GLDLKQVDGLP

In some embodiments, the two-vector split intein system of the disclosure includes a polynucleotide encoding a C-intein peptide having an amino acid sequence of SEQ ID NO: 15 or having at least 85% (e.g., at least 86%, 87%, 88%, 89%, 90%, 91%, 92%, 93%, 94%, 95%, 96%, 97%, 98%, 99%, or more) sequence identity to SEQ ID NO: 15, as is shown below.

(SEQ ID NO: 15)
MKRTADGSEFESPKKKRKVKIISRKSLGTQNVYDIGVEKDHNFLL
KNGLVASN

In some embodiments, the two-vector split intein system includes a first vector including a polynucleotide encoding an N-intein peptide having an amino acid sequence of SEQ ID NO: 14 (e.g., positioned 3′ of a polynucleotide encoding an N-terminal portion of a stereocilin protein) and a second vector including a polynucleotide encoding a C-intein polypeptide having an amino acid sequence of SEQ ID NO: 15 (e.g., positioned 5′ of a polynucleotide encoding a C-terminal portion of a stereocilin protein).

In some embodiments, the two-vector split intein system of the disclosure includes a polynucleotide encoding an N-intein peptide having an amino acid sequence of CFSGDTLVALTD (SEQ ID NO: 16). In some embodiments, the two-vector split intein system of the disclosure includes a polynucleotide encoding an N-intein peptide having an amino acid sequence of CLAGDTLITLA (SEQ ID NO: 17). In some embodiments, the two-vector split intein system of the disclosure includes a polynucleotide encoding an N-intein peptide having an amino acid sequence of CLQNGTRLLR (SEQ ID NO: 18). In some embodiments, the two-vector split intein system of the disclosure includes a polynucleotide encoding an N-intein peptide having an amino acid sequence of CLTGDSQVLTR (SEQ ID NO: 19). In some embodiments, the two-vector split intein system of the disclosure includes a polynucleotide encoding an N-intein peptide having an amino acid sequence of CLTYETEIMTV (SEQ ID NO: 20). In some embodiments, the two-vector split intein system of the disclosure includes a polynucleotide encoding an N-intein peptide having an amino acid sequence of CLSGNTKVRFRY (SEQ ID NO: 21). In some embodiments, the two-vector split intein system of the disclosure includes a polynucleotide encoding an N-intein peptide having an amino acid sequence that has least 85% (e.g., at least 86%, 87%, 88%, 89%, 90%, 91%, 92%, 93%, 94%, 95%, 96%, 97%, 98%, 99%, or more) sequence identity to any one of SEQ ID NOs: 16-21.

In some embodiments, the two-vector split intein system of the disclosure includes a polynucleotide encoding a C-intein peptide having an amino acid sequence of GVFVHN (SEQ ID NO: 22). In some embodiments, the two-vector split intein system of the disclosure includes a polynucleotide encoding a C-intein peptide having an amino acid sequence of GLLVHN (SEQ ID NO: 23). In some embodiments, the two-vector split intein system of the disclosure includes a polynucleotide encoding a C-intein peptide having an amino acid sequence of GLIASN (SEQ ID NO: 24). In some embodiments, the two-vector split intein system of the disclosure includes a polynucleotide encoding a C-intein peptide having an amino acid sequence of GLVVHN (SEQ ID NO: 25). In some embodiments, the two-vector split intein system of the disclosure includes a polynucleotide encoding a C-intein peptide having an amino acid sequence that has least 85% (e.g., at least 86%, 87%, 88%, 89%, 90%, 91%, 92%, 93%, 94%, 95%, 96%, 97%, 98%, 99%, or more) sequence identity to any one of SEQ ID NOs: 22-25.

In some embodiments, the two-vector split intein system includes a first vector including a polynucleotide encoding an N-intein peptide having an amino acid sequence of SEQ ID NO: 16 (e.g., positioned 3′ of a polynucleotide encoding an N-terminal portion of a stereocilin protein) and a second vector including a polynucleotide encoding a C-intein polypeptide having an amino acid sequence of SEQ ID NO: 22 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 19 (e.g., positioned 3′ of a polynucleotide encoding an N-terminal portion of a stereocilin protein) and a second vector including a polynucleotide encoding a C-intein polypeptide having an amino acid sequence of SEQ ID NO: 23 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 20 (e.g., positioned 3′ of a polynucleotide encoding an N-terminal portion of a stereocilin protein) and a second vector including a polynucleotide encoding a C-intein polypeptide having an amino acid sequence of SEQ ID NO: 24 (e.g., positioned 5′ of a polynucleotide encoding a C-terminal portion of a stereocilin protein). In some embodiments, the two-vector split intein system includes a first vector including a polynucleotide encoding an N-intein peptide having an amino acid sequence of SEQ ID NO: 21 (e.g., positioned 3′ of a polynucleotide encoding an N-terminal portion of a stereocilin protein) and a second vector including a polynucleotide encoding a C-intein polypeptide having an amino acid sequence of SEQ ID NO: 25 (e.g., positioned 5′ of a polynucleotide encoding a C-terminal portion of a stereocilin protein).

In some embodiments, the two-vector split intein system of the disclosure collectively includes one or more polynucleotides encoding an N-intein and C-intein pair described in Table 5 or one or more polynucleotides encoding an N-intein and C-intein pair having at least 85% sequence identity (e.g., at least 86%, 87%, 88%, 89%, 90%, 91%, 92%, 93%, 94%, 95%, 96%, 97%, 98%, 99%, or more) sequence identity to an N-intein and C-intein pair described in Table 5, below. In some embodiments, the two-vector split intein system includes a first vector including a polynucleotide encoding an N-intein peptide having an amino acid sequence listed in Table 5 (e.g., positioned 3′ of a polynucleotide encoding an N-terminal portion of a stereocilin protein) and a second vector including a polynucleotide encoding a C-intein polypeptide having an amino acid sequence listed in the same row of Table 5 as the N-intein amino acid sequence (e.g., positioned 5′ of a polynucleotide encoding a C-terminal portion of a stereocilin protein).

TABLE 5
Representative split intein sequence pairs

| | | SEQ | | SEQ |
|---|---|---|---|---|
| Intein | N-intein amino acid sequence | ID NO | C-intein amino acid sequence | ID NO |
| Npu- | CLSYETEILTVEYGLLPIGKIVEKRIE | 26 | IKIATRKYLGKQNVYDIGVERDH | 27 |
| DnaE | CTVYSVDNNGNIYTQPVAQWHDRG | | NFALKNGFIASN | |
| | EQEVFEYCLEDGSLIRATKDHKFMT | | | |
| | VDGQMLPIDEIFERELDLMRVDNLP | | | |
| | N | | | |
| Rma- | CLAGDTLITLADGRRVPIRELVSQQN | 28 | AAACPELRQLAQSDVYWDPIV | 29 |
| DnaB | FSVWALNPQTYRLERARVSRAFCT | | SIEPDGVEEVFDLTVPGPHNFV | |
| | GIKPVYRLTTRLGRSIRATANHRFLT | | ANDIIAHN | |
| | PQGWKRVDELQPGDYLALPRRIPTA | | | |
| | STPTL | | | |
| mNpu- | CLSYDTEILTVEYGILPIGKIVEKRIEC | 30 | VKVIGRRSLGVQRIFDIGLPQY | 31 |
| DnaE | TVYSVDNNGNIYTQPVAQWHDRGE | | HNFLLANGAIAAN | |
| | QEVFEYCLEDGSLIRATKDHKFMTV | | | |
| | DGQMMPIDEIFERELDLMRVDNLPN | | | |
| Cfa | CLSYDTEILTVEYGFLPIGKIVEERIE | 32 | VKIISRKSLGTQNVYDIGVEKDH | 33 |
| | CTVYTVDKNGFVYTQPIAQWHNRG | | NFLLKNGLVASN | |
| | EQEVFEYCLEDGSIIRATKDHKFMTT | | | |
| | DGQMLPIDEIFERGLDLKQVDGLP | | | |
| DnaE | CLSYDTEILTVEYGFLPIGKIVEERIE | 34 | KRTADGSEFESPKKKRKVKIIS | 35 |
| | CTVYTVDKNGFVYTQPIAQWHNRG | | RKSLGTQNVYDIGVEKDHNFLL | |
| | EQEVFEYCLEDGSIIRATKDHKFMTT | | KNGLVASN | |
| | DGQMLPIDEIFERGLDLKQVDGLP | | | |
| Ssp | CLSFGTEILTVEYGPLPIGKIVSEEIN | 36 | VKVIGRRSLGVQRIFDIGLPQD | 37 |
| DnaE | CSVYSVDPEGRVYTQAIAQWHDRG | | HNFLLANGAIAANC | |
| | EQEVLEYELEDGSVIRATSDHRFLTT | | | |
| | DYQLLAIEEIFARQLDLLTLENIKQTE | | | |
| | EALDNHRLPFPLLDAGTIK | | | |
| DnaE | CLSYDTEILTVEYGFLPIGKIVEERIE | 38 | GLPVKIISRKSLGTQNVYDIGVE | 39 |
| | CTVYTVDKNGFVYTQPIAQWHNRG | | KDHNFLLKNGLVASN | |
| | EQEVFEYCLEDGSIIRATKDHKFMTT | | | |
| | DGQMLPIDEIFERGLDLQVD | | | |
| Gp41.1 | CLDLKTQVQTPQGMKEISNIQVGDL | 49 | MMLKKILKIEELDERELIDIEVSG | 50 |
| | VLSNTGYNEVLNVFPKSKKKSYKITL | | NHLFYANDILTHN | |
| | EDGKEIICSEEHLFPTQTGEMNISGG | | | |
| | LKEGMCLYVKE | | | |
| Gp41.8 | CLSLDTMVVTNGKAIEIRDVKVGDW | 51 | MCEIFENEIDWDEIASIEYVGVE | 52 |
| | LESECGPVQVTEVLPIIKQPVFEIVLK | | ETIDINVTNDRLFFANGILTHN | |
| | SGKKIRVSANHKFPTKDGLKTINSGL | | | |
| | KVGDFLRSRAK | | | |
| NrdJ1 | CLVGSSEIITRNYGKTTIKEVVEIFDN | 53 | MEAKTYIGKLKSRKIVSNEDTY | 54 |
| | DKNIQVLAFNTHTDNIEWAPIKAAQL | | DIQTSTHNFFANDILVHN | |
| | TRPNAELVELEINTLHGVKTIRCTPD | | | |
| | HPVYTKNRDYVRADELTDDDELVVA | | | |
| IMPDH1 | CFVPGTLVNTENGLKKIEEIKVGDKV | 55 | MKFKLKEITSIETKHYKGKVHDL | 56 |
| | FSHTGKLQEVVDTLIFDRDEEIISING | | TVNQDHSYNVRGTVVHN | |
| | IDCTKNHEFYVIDKENANRVNEDNIH | | | |
| | LFARWVHAEELDMKKHLLIELE | | | |
| NrdA2 | CLTGDAKIDVLIDNIPISQISLEEVVNL | 57 | MGLKIIKRESKEPVFDITVKDNS | 58 |
| | FNEGKEIYVLSYNIDTKEVEYKEISD | | NFFANNILVHN | |
| | AGLISESAEVLEIIDEETGQKIVCTPD | | | |
| | HKVYTLNRGYVSAKDLKEDDELVFS | | | |

The Npu N-intein of SEQ ID NO: 26 may be encoded by a polynucleotide having the DNA sequence of SEQ ID NO: 40, as is shown below.

(SEQ ID NO: 40)
TGCCTGAGCTACGAGACCGAGATCCTGACCGTGGAGTACGGCCTG
CTGCCCATCGGCAAGATCGTGGAGAAGAGAATCGAGTGCACCGTG
TACAGCGTGGACAACAACGGCAACATCTACACCCAGCCCGTGGCC
CAGTGGCACGACAGAGGCGAGCAGGAGGTGTTCGAGTACTGCCTG
GAGGACGGCAGCCTGATCAGAGCCACCAAGGACCACAAGTTCATG
ACCGTGGACGGCCAGATGCTGCCCATCGACGAGATCTTCGAGAGA
GAGCTGGACCTGATGAGAGTGGACAACCTGCCCAAC

The Npu C-intein of SEQ ID NO: 27 may be encoded by a polynucleotide having the DNA sequence of SEQ ID NO: 41, as is shown below.

(SEQ ID NO: 41)
ATCAAGATCGCCACAAGAAAGTACCTGGGCAAGCAGAACGTGTAC
GACATCGGCGTGGAGAGAGACCACAACTTCGCCCTGAAGAACGGC
TTCATCGCCAGCAAT

A split intein of the disclosure (i.e., the N-intein and C-intein) can include nucleophile amino acid at or near its N- or C-terminus that is capable of performing the trans-splicing reaction. In some embodiments, the nucleophile amino acid is selected from serine, threonine, cysteine, or alanine.

In some embodiments, the first vector and/or the second vector further include one or more additional regulatory sequences, such as, e.g., a WPRE sequence, an enhancer sequence, a poly(A) sequence, a terminator sequence, or a degradation signal, among others.

In some embodiments, the split intein system described herein includes a ligand-dependent intein, which performs protein splicing upon contact with a ligand (e.g., small molecules such as 4-hydroxytamoxifen, peptides, proteins, polynucleotides, amino acids, nucleotides, etc.). Various ligand-dependent inteins are described in US 2014/0065711, the disclosure of which is incorporated by reference herein as it relates to ligand-dependent inteins.

The present disclosure provides vectors containing one or more degradation signals within the intein (e.g., N-intein or C-intein) polypeptide(s) that mediate protein degradation by the ubiquitin-proteasome system and/or autophagy-lysosome pathways. Such sequences may be incorporated into the vector systems of the disclosure to avoid or reduce accumulation of excised intein proteins within target cells. Exemplary degradation signals include N-degrons and C-degrons, which are peptide sequences containing motifs containing lysine residues capable of polyubiquitylation and subsequent targeting for degradation. In some embodiments, degrons are degradation signals located within an intein that are not at the N-terminus nor the C-terminus of the intein. In some embodiments, the N-intein protein includes one or more (e.g., 2, 3, 4, 5, or more) degrons. In some embodiments, the C-intein protein includes one or more (e.g., 2, 3, 4, 5, or more) degrons. In some embodiments, the degron is a CL1 degron, which is a C-terminal destabilizing peptide that shares structural similarity with misfolded proteins and is recognized by the ubiquitination system. In some embodiments, the degron is a PB29, SMN, CIITA, or ODC degron. Such degradation signals are described in WO 2016/13932, which is incorporated by reference herein as it relates to degradation signals. Another example of a degradation signal includes the E. coli dihydrofolate reductase (ecDHFR)-derived degron, as is described in WO 2020/079034 (incorporated by reference herein). Additional degradation signals include FKBP12 degradation domains (Banaszynski et al., Cell 126:995-1004, 2006), PEST degradation domains (Rechsteiner and Rogers, Trends Biochem Sci. 21:267-271, 1996), UbR tag ubiquitination signals (Chassin et al., Nat Commun. 10:2013, 2019), and destabilized mutations of human ELRBD (Miyazaki et al., J. Am. Chem. Soc., 134:3942-3945, 2012).

### Expression of Exogenous Polynucleotides in Mammalian Cells

Mutations in a variety of genes, such as MYO7A, POU4F3, SLC17A8, and TMC1, have been linked to sensorineural hearing loss, and some of these mutations, such as mutations in MYO7A, are also associated with vestibular dysfunction. The compositions and methods described herein can be used to induce or increase the expression of proteins encoded by genes of interest (e.g., the wild-type form of genes implicated in hearing loss and/or vestibular dysfunction, or genes involved in hair cell development, function, cell fate specification, regeneration, survival, or maintenance) specifically in hair cells (e.g., hair cells that express endogenous STRC, such as cochlear hair cells (e.g., outer hair cells and inner hair cells) and vestibular hair cells (e.g., type I and type II vestibular hair cells)) by administering a nucleic acid vector that contains an STRC promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide sequence that encodes a protein of interest. A wide array of methods has been established for the delivery of proteins to mammalian cells and for the stable expression of genes encoding proteins in mammalian cells.

Proteins that can be expressed in connection with the compositions described herein (e.g., when a transgene encoding the protein is operably linked to an STRC promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) are proteins that are expressed in healthy hair cells (e.g., cochlear and/or vestibular hair cells, e.g., proteins that play a role in hair cell development, function, regeneration, cell fate specification, survival, or maintenance, or proteins that are deficient in subjects with sensorineural hearing loss or vestibular dysfunction) or other therapeutic proteins of interest. Proteins that can be expressed in hair cells using the compositions and methods described herein include ACTG1, FSCN2, RDX, POU4F3, TRIOBP, TPRN, XIRP2, ATOH1, GFI1, CHRNA9, CHRNA10, CIB3, CDH23, PCDH15, KNCN, DFNB59, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, EPS8, EPS8L2, ESPN, ESPNL, PRPH2, SLC8A2, ZCCHC12, LRTOMT2, LRTOMT1, USH1C, SLC26A5, PIEZO2, ELFN1, TTC24, DYTN, CCER2, LRTM2, KCNA10, CLRN1, CLRN2, SKOR1, TCTEX1D1, FCRLB, GRXCR2, SERPINE3, NHLH1, HSP70, HSP90, ATF6, PERK, IRE1, WHRN, OCM, ISL1, TMTC4, BIP, and KCNQ4. The STRC promoters described herein can also be used to express a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA in hair cells (e.g., cochlear or vestibular hair cells). In addition, the STRC promoters described herein can be operably linked to a polynucleotide encoding an N-terminal portion of a stereocilin protein (e.g., a wild-type stereocilin protein, such as an N-terminal portion of the protein of SEQ ID NO: 3 or SEQ ID NO: 4) for use in a two-vector system described herein,

### Polynucleotides Encoding Proteins of Interest

One platform that can be used to achieve therapeutically effective intracellular concentrations of proteins of interest in mammalian cells is via the stable expression of the gene encoding the protein of interest (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Proteins of interest can also be introduced into a mammalian cell by targeting a vector containing a gene encoding a protein of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a protein of interest by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith, et al., Mol. Sys. Biol., 3:73, online publication, the disclosure of which is incorporated herein by reference. The promoter used in the methods and compositions described herein is an STRC promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48).

Once a polynucleotide encoding a protein of interest has been incorporated into a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a protein of interest and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. An enhancer may be spliced into a vector containing a polynucleotide encoding a protein of interest, for example, at a position 5′ or 3′ to this gene. In a preferred orientation, the enhancer is positioned at the 5′ side of the promoter, which in turn is located 5′ relative to the polynucleotide encoding a protein of interest.

Without being bound by theory, the inventors believe that the nucleotide sequence linking the 3′ end of the STRC promoter and the 5′ start site (ATG) of the polynucleotide encoding the protein of interest may play a role in the expression of the protein of interest. In some embodiments, that linking sequence includes a Kozak sequence or a portion thereof. In some embodiments, that linking sequence includes a multiple cloning site or a portion thereof that was utilized to insert the STRC promoter and/or the coding sequence of the protein of interest into the vector.

The nucleic acid vectors containing an STRC promoter described herein may include a Woodchuck Posttranscriptional Regulatory Element (WPRE). The WPRE acts at the mRNA level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo.

In some embodiments, the nucleic acid vectors containing an STRC promoter described herein include a reporter sequence, which can be useful in verifying the expression of a gene operably linked to an STRC promoter, for example, in cells and tissues (e.g., in cochlear hair cells, such as outer hair cells and inner hair cells, and vestibular hair cells, such as type I and type II vestibular hair cells). Reporter sequences that may be provided in a transgene include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as an STRC promoter, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer.

### Methods for the Delivery of Exogenous Polynucleotides to Target Cells

Techniques that can be used to introduce a transgene, such as a transgene operably linked to an STRC promoter described herein, into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous polynucleotides. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of polynucleotides into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of polynucleotides into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous polynucleotides, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign polynucleotides include contacting a cell with a cationic polymer-polynucleotide complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of polynucleotides. This technology is described in detail, for instance, in US 2010/0227406, the disclosure of which is incorporated herein by reference.

Another useful tool for inducing the uptake of exogenous polynucleotides by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver polynucleotides to target cells. The magnetofection principle is to associate polynucleotides with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous polynucleotides by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for Delivery of Exogenous Polynucleotides to Target Cells

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, MA, 2006). Expression vectors for use in the compositions and methods described herein contain an STRC promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide encoding a desired expression product (e.g., a polynucleotide that encodes a protein of interest or that can be transcribed to produce an inhibitory RNA), as well as, e.g., additional sequence elements used for the expression of these agents and/or the integration of these polynucleotide sequences into the genome of a mammalian cell. Vectors that can contain an STRC promoter operably linked to polynucleotide encoding a desired expression product (e.g., a transgene encoding a protein of interest) include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of a desired expression product (e.g., a protein of interest) include plasmids that contain regulatory sequences, such as enhancer regions, which direct gene transcription. Other useful vectors for expression of a desired expression product (e.g., a protein of interest) contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Polynucleotide Delivery

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Polynucleotide Delivery

In some embodiments, polynucleotides of the compositions and methods described herein are incorporated into rAAV vectors and/or virions in order to facilitate their introduction into a cell. rAAV vectors useful in the compositions and methods described herein are recombinant polynucleotide constructs that include (1) an STRC promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48), (2) a sequence to be expressed (e.g., a polynucleotide encoding a heterologous expression product or a portion of a stereocilin protein), and (3) viral sequences that facilitate integration and expression of the sequence to be expressed. The viral sequences may include those sequences of AAV that are required in cis for replication and packaging (e.g., functional ITRs) of the DNA into a virion. In typical applications, the sequence to be expressed encodes a protein that can promote hair cell development, hair cell function, hair cell regeneration, hair cell fate specification, hair cell survival, or hair cell maintenance, or a wild-type form of a hair cell protein that is mutated in subjects with forms of hereditary hearing loss or vestibular dysfunction that may be useful for improving hearing or vestibular function in subjects carrying mutations that have been associated with hearing loss, deafness, or vestibular dysfunction (e.g., dizziness, vertigo, imbalance, bilateral vestibulopathy, oscillopsia, or a balance disorder). Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., an STRC promoter operably linked to a transgene encoding a protein of interest or to a polynucleotide encoding a portion of a WT stereocilin protein) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, Anc80, Anc80L65, DJ/8, DJ/9, 7m8, PHP.B, PHP.eb, and PHP.S. For targeting hair cells, AAV1, AAV2, AAV2quad(Y-F), AAV6, AAV8, AAV9, Anc80, Anc80L65, DJ/9, 7m8, and PHP.B may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV2quad(Y-F), AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The STRC promoters described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) may be operably linked to a polynucleotide encoding a desired expression product (e.g., a transgene encoding a protein of interest) or to a polynucleotide that encodes a portion of a stereocilin protein and incorporated into a vehicle for administration into a patient, such as a human patient suffering from sensorineural hearing loss and/or vestibular dysfunction. Pharmaceutical compositions containing vectors, such as viral vectors, that contain an STRC promoter described herein operably linked to a polynucleotide encoding a desired expression product can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of nucleic acid vectors (e.g., viral vectors) containing an STRC promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a polynucleotide encoding a desired expression product or to a polynucleotide that encodes a portion of a stereocilin protein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing sensorineural hearing loss and/or vestibular dysfunction by a variety of routes, such as local administration to the middle or inner ear (e.g., administration into the perilymph or endolymph, such as to or through the oval window, round window, or semicircular canal (e.g., the horizontal canal), or by transtympanic or intratympanic injection, e.g., administration to a hair cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly). In embodiments related to two-vector systems, the first and second nucleic acid vectors can be administered simultaneously (e.g., in one composition) or sequentially (e.g., one nucleic acid vector is administered immediately after the other nucleic acid vector, or 5 minutes, 10 minutes, 15 minutes, 20 minutes, 25 minutes, 30 minutes, 45 minutes, 1 hour, 2 hours, or more after the first nucleic acid vector). The first and second nucleic acid vector can have the same capsid or different capsids (e.g., AAV capsids).

Subjects that may be treated as described herein are subjects having or at risk of developing sensorineural hearing loss and/or vestibular dysfunction (e.g., subjects having or at risk of developing hearing loss, vestibular dysfunction, or both). The compositions and methods described herein can be used to treat subjects having or at risk of developing damage to cochlear hair cells (e.g., damage related to acoustic trauma, disease or infection, head trauma, ototoxic drugs, or aging), subjects having or at risk of developing damage to vestibular hair cells (e.g., damage related to disease or infection, head trauma, ototoxic drugs, or aging), subjects having or at risk of developing sensorineural hearing loss, deafness, or auditory neuropathy, subjects having or at risk of developing vestibular dysfunction (e.g., dizziness, vertigo, imbalance, bilateral vestibulopathy, oscillopsia, or a balance disorder), subjects having tinnitus (e.g., tinnitus alone, or tinnitus that is associated with sensorineural hearing loss or vestibular dysfunction), subjects having a genetic mutation associated with hearing loss and/or vestibular dysfunction, or subjects with a family history of hereditary hearing loss, deafness, auditory neuropathy, tinnitus, or vestibular dysfunction. In some embodiments, the disease associated with damage to or loss of hair cells (e.g., cochlear and/or vestibular hair cells) is an autoimmune disease or condition in which an autoimmune response contributes to hair cell damage or death. Autoimmune diseases linked to sensorineural hearing loss and vestibular dysfunction include autoimmune inner ear disease (AIED), polyarteritis nodosa (PAN), Cogan's syndrome, relapsing polychondritis, systemic lupus erythematosus (SLE), Wegener's granulomatosis, Sjögren's syndrome, and Behçet's disease. Some infectious conditions, such as Lyme disease and syphilis can also cause hearing loss and vestibular dysfunction (e.g., by triggering autoantibody production). Viral infections, such as rubella, cytomegalovirus (CMV), lymphocytic choriomeningitis virus (LCMV), HSV types 1 &2, West Nile virus (WNV), human immunodeficiency virus (HIV) varicella zoster virus (VZV), measles, and mumps, can also cause hearing loss and vestibular dysfunction. In some embodiments, the subject has or is at risk of developing hearing loss and/or vestibular dysfunction that is associated with or results from loss of hair cells (e.g., cochlear or vestibular hair cells). The STRC two-vector systems described herein can be used to treat subjects having a mutation in STRC (e.g., a mutation that reduces stereocilin function or expression, or a STRC mutation associated with sensorineural hearing loss or vestibular dysfunction, such as a mutation that causes nonsyndromic hearing loss, e.g., DFNB16), including subjects having a mutation in STRC who exhibit symptoms of hearing loss or vestibular dysfunction and subjects who do not yet present with symptoms (e.g., preventative treatment of subjects having a mutation in STRC), or subjects whose STRC mutational status and/or STRC activity level is unknown. The methods described herein may include a step of screening a subject for one or more mutations in genes known to be associated with hearing loss and/or vestibular dysfunction prior to treatment with or administration of the compositions described herein. A subject can be screened for a genetic mutation using standard methods known to those of skill in the art (e.g., genetic testing). The methods described herein may also include a step of assessing hearing and/or vestibular function in a subject prior to treatment with or administration of the compositions described herein. Hearing can be assessed using standard tests, such as audiometry, auditory brainstem response (ABR), electrocochleography (ECOG), and otoacoustic emissions. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess hearing and/or vestibular function in a subject after treatment with or administration of the compositions described herein. The compositions and methods described herein may also be administered as a preventative treatment to patients at risk of developing hearing loss and/or vestibular dysfunction, e.g., patients who have a family history of hearing loss or vestibular dysfunction (e.g., inherited hearing loss or vestibular dysfunction), patients carrying a genetic mutation associated with hearing loss or vestibular dysfunction who do not yet exhibit hearing impairment or vestibular dysfunction, or patients exposed to risk factors for acquired hearing loss (e.g., acoustic trauma, disease or infection, head trauma, ototoxic drugs, or aging) or vestibular dysfunction (e.g., disease or infection, head trauma, ototoxic drugs, or aging). The compositions and methods described herein can also be used to treat a subject with idiopathic vestibular dysfunction.

The compositions and methods described herein can be used to induce or increase hair cell regeneration in a subject (e.g., cochlear and/or vestibular hair cell regeneration). Subjects that may benefit from compositions that induce or increase hair cell regeneration include subjects suffering from hearing loss or vestibular dysfunction as a result of loss of hair cells (e.g., loss of hair cells related to trauma (e.g., acoustic trauma or head trauma), disease or infection, ototoxic drugs, or aging), and subjects with abnormal hair cells (e.g., hair cells that do not function properly when compared to normal hair cells), damaged hair cells (e.g., hair cell damage related to trauma (e.g., acoustic trauma or head trauma), disease or infection, ototoxic drugs, or aging), or reduced hair cell numbers due to genetic mutations or congenital abnormalities. The compositions and methods described herein can also be used to promote or increase hair cell survival (e.g., increase survival of damaged hair cells, promote repair of damaged hair cells, or preserve hair cells in a subject at risk of loss of hair cells (e.g., loss of hair cells due to age, exposure to loud noise, disease or infection, head trauma, or ototoxic drugs)). The compositions and methods described herein can also be used to promote or increase hair cell maturation, improve hair cell structure (e.g., improve stereocilia bundle morphology), or to improve hair cell function (e.g., improve stereocilia bundle deflection), which can lead to improved auditory and/or vestibular function.

The compositions and methods described herein can also be used to prevent or reduce hearing loss and/or vestibular dysfunction caused by ototoxic drug-induced hair cell damage or death (e.g., cochlear hair cell and/or vestibular hair cell damage or death) in subjects who have been treated with ototoxic drugs, or who are currently undergoing or soon to begin treatment with ototoxic drugs. Ototoxic drugs are toxic to the cells of the inner ear, and can cause sensorineural hearing loss, vestibular dysfunction (e.g., vertigo, dizziness, imbalance, bilateral vestibulopathy (bilateral vestibular hypofunction), or oscillopsia), tinnitus, or a combination of these symptoms. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods and compositions described herein can be used to treat bilateral vestibulopathy (bilateral vestibular hypofunction) or oscillopsia. Bilateral vestibulopathy (bilateral vestibular hypofunction) and oscillopsia can be induced by aminoglycosides (e.g., the methods and compositions described herein can be used to promote or increase hair cell regeneration in a subject having or at risk of developing aminoglycoside-induced bilateral vestibulopathy (bilateral vestibular hypofunction) or oscillopsia).

The polynucleotide encoding a desired expression product operably linked to an STRC promoter (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) for treatment of a subject as described herein can be a transgene that encodes a protein expressed in healthy hair cells (e.g., a protein that plays a role in hair cell development, hair cell function, hair cell fate specification, hair cell regeneration, hair cell survival, or hair cell maintenance, or a protein that is deficient in a subject with sensorineural hearing loss or vestibular dysfunction), a transgene that encodes another protein of interest (e.g., a reporter protein, such as a fluorescent protein, lacZ, or luciferase), or a polynucleotide that can be transcribed to produce an shRNA, an ASO, a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA. The transgene may be selected based on the cause of the subject's hearing loss or vestibular dysfunction (e.g., if the subject's hearing loss or vestibular dysfunction is associated with a particular genetic mutation, the transgene can be a wild-type form of the gene that is mutated in the subject, or if the subject has hearing loss and/or vestibular dysfunction associated with loss of hair cells, the transgene can encode a protein that promotes hair cell regeneration), the severity of the subject's hearing loss or vestibular dysfunction, the health of the subject's hair cells, the subject's age, the subject's family history of hearing loss or vestibular dysfunction, or other factors. The proteins that may be expressed by a transgene operably linked an STRC promoter for treatment of a subject as described herein include ACTG1, FSCN2, RDX, POU4F3, TRIOBP, TPRN, XIRP2, ATOH1, GFI1, CHRNA9, CHRNA10, CIB3, CDH23, PCDH15, KNCN, DFNB59, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, EPS8, EPS8L2, ESPN, ESPNL, PRPH2, SLC8A2, ZCCHC12, LRTOMT2, LRTOMT1, USH1C, SLC26A5, PIEZO2, ELFN1, TTC24, DYTN, CCER2, LRTM2, KCNA10, CLRN1, CLRN2, SKOR1, TCTEX1D1, FCRLB, GRXCR2, SERPINE3, NHLH1, HSP70, HSP90, ATF6, PERK, IRE1, WHRN, OCM, ISL1, TMTC4, BIP, and KCNQ4. A polynucleotide encoding an N-terminal portion of stereocilin can also be operably linked to an STRC promoter described herein (e.g., in a first vector in a two-vector system). In some embodiments, a polynucleotide encoding a fusion protein containing a C-intein and a C-terminal portion of stereocilin can be operably linked to a STRC promoter described herein (e.g., in a second vector in an intein expression system).

Treatment may include administration of a composition containing a nucleic acid vector (e.g., an AAV vector) containing an STRC promoter described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear (e.g., the cochlea and/or vestibular system). In cases in which the nucleic acid vectors are AAV vectors (e.g., AAV1, AAV2, AAV2quad(Y-F), AAV3, AAV4, AAV5, AAV6, AAV7, AAV8, AAV9, AAV10, rh10, rh39, rh43, rh74, Anc80, Anc80L65, DJ/8, DJ/9, 7m8, PHP.B, PHP.eb, or PHP.S vectors), the viral vectors may be administered to the patient at a dose of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein are administered in an amount sufficient to improve hearing, improve vestibular function (e.g., improve balance or reduce dizziness or vertigo), reduce tinnitus, treat bilateral vestibulopathy, treat oscillopsia, treat a balance disorder, increase or induce expression of a protein encoded by a transgene operably linked to an STRC promoter in hair cells, increase function of a protein encoded by a transgene operably linked to an STRC promoter in hair cells, prevent or reduce hair cell damage (e.g., hair cell damage related to acoustic trauma, head trauma, ototoxic drugs, disease or infection, or aging), prevent or reduce hair cell death (e.g., ototoxic drug-induced hair cell death, noise-related hair cell death, age-related hair cell death, disease or infection-related hair cell death, or head trauma-related hair cell death), promote or increase hair cell development, increase hair cell numbers, induce or increase hair cell regeneration (e.g., cochlear and/or vestibular hair cell regeneration), promote or increase hair cell survival, promote or increase hair cell maturation, improve hair bundle attachment to the tectorial membrane (e.g., OHC hair bundle attachment), improve hair cell structure, or improve hair cell function. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. Vestibular function may be evaluated using standard tests for balance and vertigo (e.g., eye movement testing (e.g., ENG or VNG), posturography, VOR testing (e.g., head impulse testing (Halmagyi-Curthoys testing, e.g., VHIT), or caloric reflex testing), rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss and/or vestibular dysfunction (e.g., in subjects who carry a genetic mutation associated with hearing loss or vestibular dysfunction, who have a family history of hearing loss or vestibular dysfunction (e.g., hereditary hearing loss or vestibular dysfunction), or who have been exposed to risk factors associated with hearing loss or vestibular dysfunction (e.g., ototoxic drugs, head trauma, disease or infection, or acoustic trauma) but do not exhibit hearing impairment or vestibular dysfunction (e.g., vertigo, dizziness, or imbalance), or in subjects exhibiting mild to moderate hearing loss or vestibular dysfunction). Expression of a protein encoded by a transgene operably linked to an STRC promoter in the nucleic acid vector administered to the subject may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100%, 125%, 150%, 200% or more) compared to expression prior to administration of the compositions described herein. Hair cell numbers, hair cell function, hair cell regeneration, or function of a protein encoded by a transgene operably linked to an STRC promoter in the nucleic acid vector administered to the subject may be evaluated indirectly based on hearing tests or tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to hair cell numbers, hair cell function, hair cell regeneration, or function of the protein prior to administration of the compositions described herein. Hair cell damage or death may be reduced by 5% or more (e.g., 5%, 10%, 15%, 20%, 30%, 40%, 50%, 60%, 70%, 80%, 90%, 100%, 125%, 150%, 200% or more) compared to hair cell damage and death typically observed in untreated subjects. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in treating sensorineural hearing loss and/or vestibular dysfunction. Compositions may include an STRC promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48), a nucleic acid vector containing such a polynucleotide, a nucleic acid vector containing an STRC promoter described herein operably linked to polynucleotide encoding a desired expression product (e.g., a transgene encoding a protein of interest, such as a protein that can be expressed in hair cells to treat hearing loss and/or vestibular dysfunction), and a nucleic acid vector system including an STRC promoter described herein (e.g., a two-vector system described herein). The nucleic acid vector may be packaged in an AAV virus capsid (e.g., AAV1, AAV2, AAV2quad(Y-F), AAV6, AAV8, AAV9, Anc80, Anc80L65, DJ/9, 7m8, or PHP.B). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. STRC Promoter-Driven GFP Expression is Enriched in Outer Hair Cells in the Organ of Corti and Restricted to Hair Cells in the Utricle in Mice

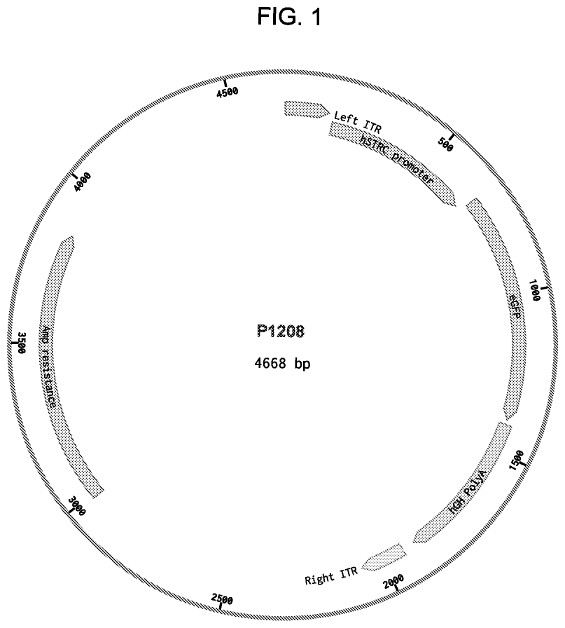

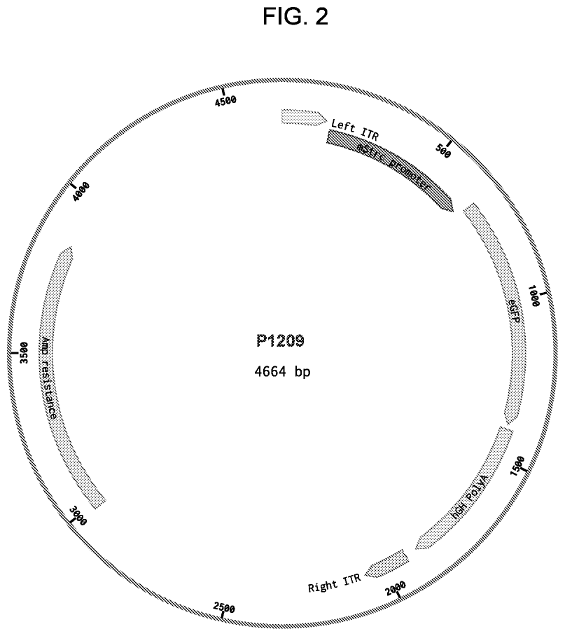

To test the specificity of the STRC promoters disclosed herein in vivo, we injected wildtype C57BL/6J mice with an AAV vector including eGFP operatively linked to certain of the disclosed STRC promoters. An AAV vector expressing eGFP under control of the STRC promoter of SEQ ID NO: 1 was prepared from transgene plasmid P1208 (FIG. 1) using well-known methods for AAV vector preparation. An AAV vector expressing eGFP under control of the STRC promoter of nucleotides 35-530 of SEQ ID NO: 2 was prepared from transgene plasmid P1209 (FIG. 2) using well-known methods for AAV vector preparation.

Early postnatal mice, two to three days after birth, were injected with 1 μl of one of the two AAV vectors (1.6×10¹⁰ vg/ear for the AAV vector expressing eGFP under control of the STRC promoter of nucleotides 35-530 of SEQ ID NO: 2; 1.1×10¹⁰ vg/ear for the AAV vector expressing eGFP under control of the STRC promoter of SEQ ID NO: 1) via a fenestration in the posterior semicircular canal.

Adult mice, six weeks of age, were injected with 2 μl of one of the two AAV vectors (3.2×10¹⁰ vg/ear for the AAV vector expressing eGFP under control of the STRC promoter of nucleotides 35-530 of SEQ ID NO: 2; 2.2×10¹⁰ vg/ear for the AAV vector expressing eGFP under control of the STRC promoter of SEQ ID NO: 1) via a fenestration in the posterior semicircular canal.

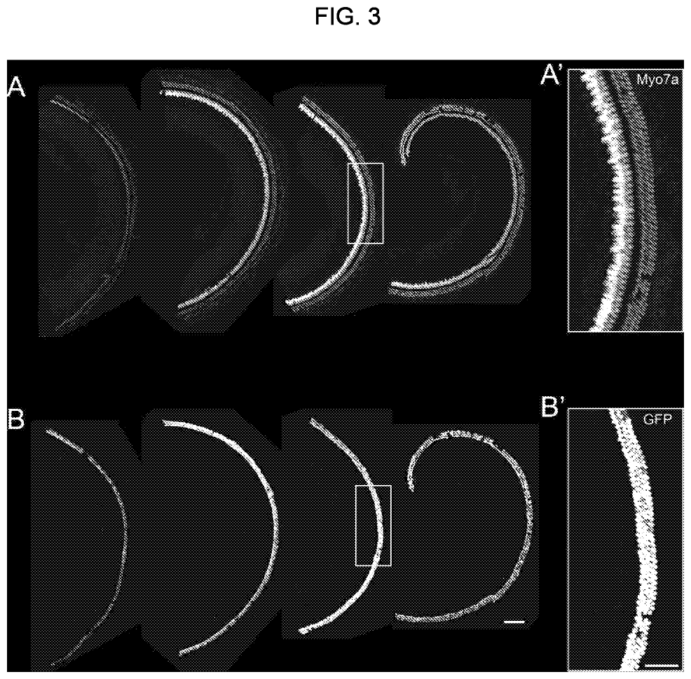

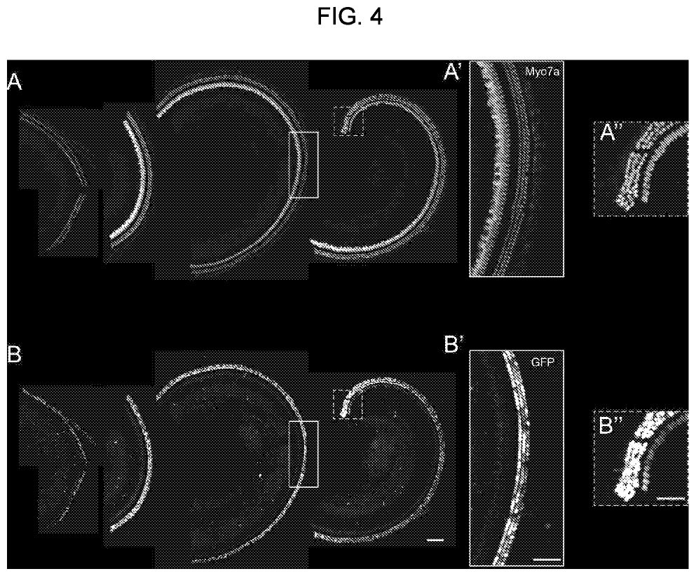

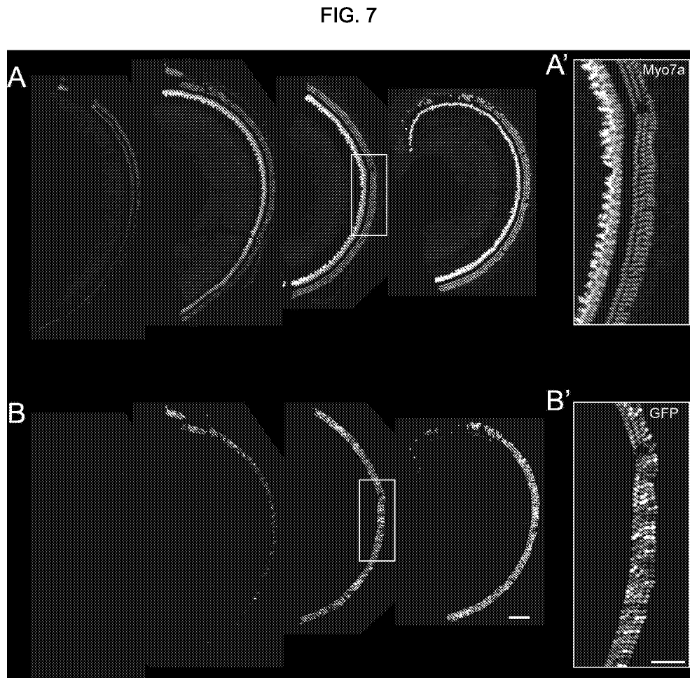

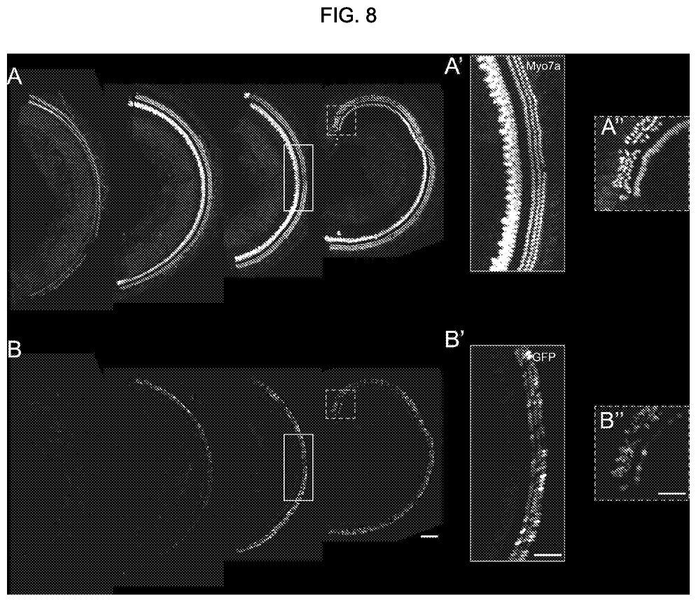

After four weeks in life neonatally injected animals were sacrificed and fixed in 10% NBF via cardiac perfusion, their temporal bones were harvested and kept in 10% NBF for additional 16 hours. For adult mice, the animals were sacrificed two weeks post-injection and the same procedure was used to harvest temporal bones. After two days decalcification in 8% EDTA, ears were micro dissected and utricles and organs of Corti prepared for immunohistochemistry. Whole mount tissue preparations of the organ of Corti were counterstained with Myosin7a antibody to visualize all hair cells (FIG. 3, panels A and A′; FIG. 4 panels, A, A′ and A″; FIG. 7, panels A and A′; FIG. 8 panels, A, A′ and A″) and imaged together with the virally mediated native GFP expression (FIG. 3, panels B and B′; FIG. 4 panels, B, B′ and B″; FIG. 7, panels B and B′; FIG. 8 panels, B, B′ and B″) under a confocal microscope (Leica SP8, 20×/0.75 NA, 2 μm step size at 2 AU).

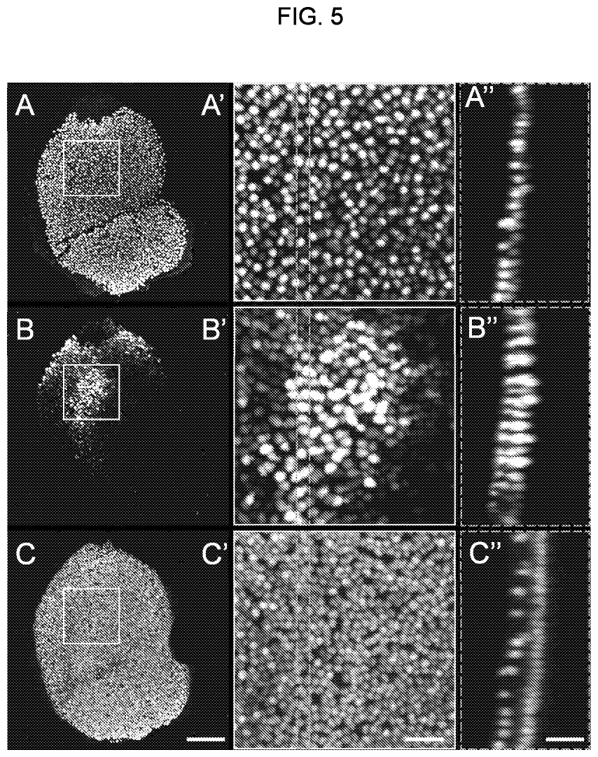

Whole mount preparations of the neonatal utricles were counterstained with Pou4f3 antibody to label all hair cell nuclei (FIG. 5, panels A, A′ and A″) and Sox2 antibody was used to label all supporting cells and type II hair cell nuclei (FIG. 5, panels C, C′ and C″) and imaged together with the virally mediated native GFP expression (FIG. 5, panels, B, B′ and B″) under a confocal microscope (Leica SP8 20×/0.75 NA, 1.5 μm step size at 2 AU). STRC promoter-driven GFP expression was significantly enriched in outer hair cells in the organ of Corti and restricted to hair cells in the utricle. Limited GFP expression was also detected in some inner hair cells in the organ of Corti.

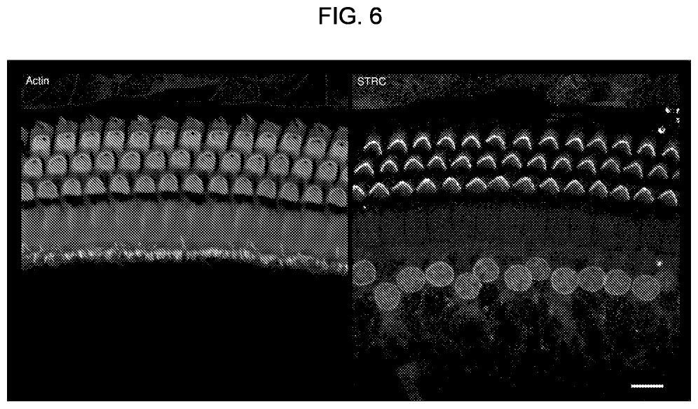

To visualize native expression of stereocilin in mice, adult CBA/CaJ mice were sacrificed and fixed in 10% NBF via cardiac perfusion and their temporal bones harvested and kept in 10% NBF for additional 16 hours. After two days decalcification in 8% EDTA organs of Corti were micro dissected and stained with an antibody against stereocilin and counterstained with Phalloidin, labelling filamentous actin in the stereocilia. Images were acquired using a confocal microscope (Zeiss LSM800, 63×/1.4 NA, 0.38 μm step size at 1 AU) (FIG. 6).

### Example 2. STRC Promoter-Driven GFP Expression is Enriched in Outer Hair Cells in the Organ of Corti and Restricted to Hair Cells in Non-Human Primates

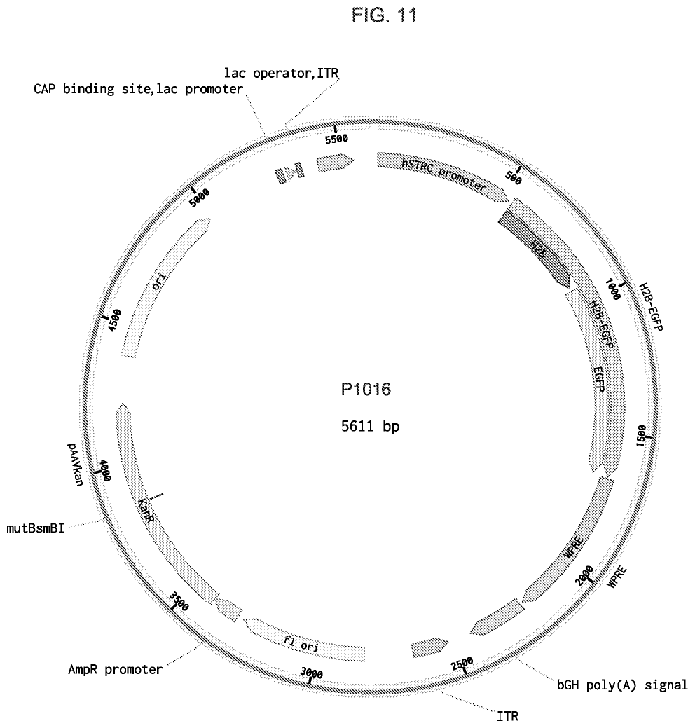

To test the specificity of the STRC promoters disclosed herein in vivo, we injected non-human primate (Macaca fascicularis) ears with an AAV vector including nuclear targeted H2B-eGFP operably linked to certain of the disclosed STRC promoters. An AAV vector expressing nuclear H2B-eGFP under control of the STRC promoter of SEQ ID NO: 1 was prepared from transgene plasmid P1016 (FIG. 11) using well-known methods for AAV vector preparation. Adult non-human primates were injected with 40 μl of vector (3.41×10¹³ vg/ml for the AAV vector expressing H2B-eGFP under control of the STRC promoter of SEQ ID NO: 1; via the round window membrane, a fenestration in the lateral semicircular canal allowed for efflux of perilymph during the procedure).

After four weeks in life animals were sacrificed and fixed in 10% NBF via cardiac perfusion, their temporal bones were harvested and kept in 10% NBF for additional 4-10 days. After six weeks decalcification in 14% EDTA, ears were micro dissected and utricles and organs of Corti prepared for immunohistochemistry, or ears were decalcified in formic acid for 6 days and paraffin embedded and sectioned in 5 μm slices.

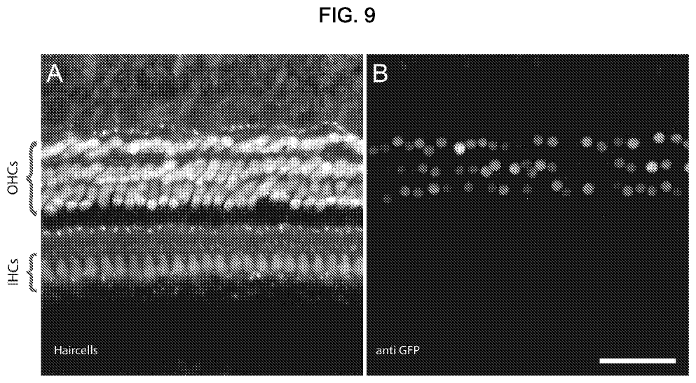

Whole mount tissue preparations of the organ of Corti were counterstained with Pou4f3 antibody to visualize all hair cells (FIG. 9, panel A) and imaged together with the virally mediated GFP expression counterstained with an antibody against GFP (FIG. 9, panel B) under a confocal microscope (Zeiss LSM880, 40×/NA 0.95, 1 μm step size at 2 AU).

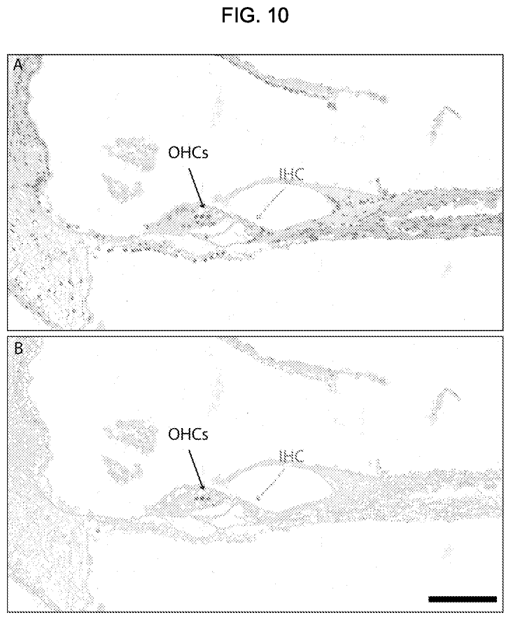

Sections were labeled with an antibody against GFP and stained with a secondary antibody conjugated to alkaline phosphatase; a red, chromatic staining was developed by the reaction of the fast red dye with the alkaline phosphatase of the secondary antibody. Sections were counterstained with Hematoxylin in blue to visualize all nuclei and imaged on a color camera at 20× magnification and converted to greyscale (FIG. 10, panel A). To visualize the red signal of the chromatic anti-GFP staining, all blue (Hematoxylin) color was extracted from the color images using Image processing software GIMP utilizing the select color tool, before converting to greyscale. Only nuclei with red signal nuclear H2B-GFP remained visible (FIG. 10, panel B).

### Example 3. An Anc80-CMV-mStrc Overlapping Dual Vector System Rescued Hearing in Stereocilin Deficient Mice

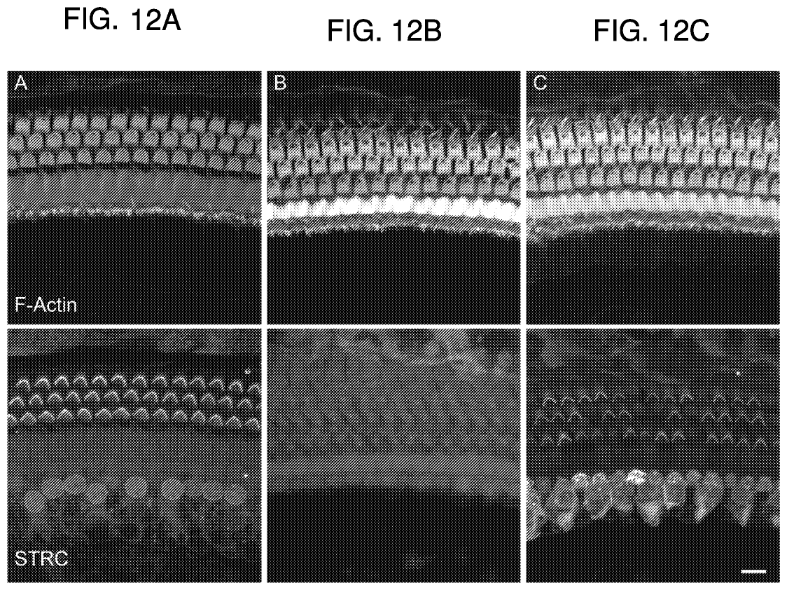

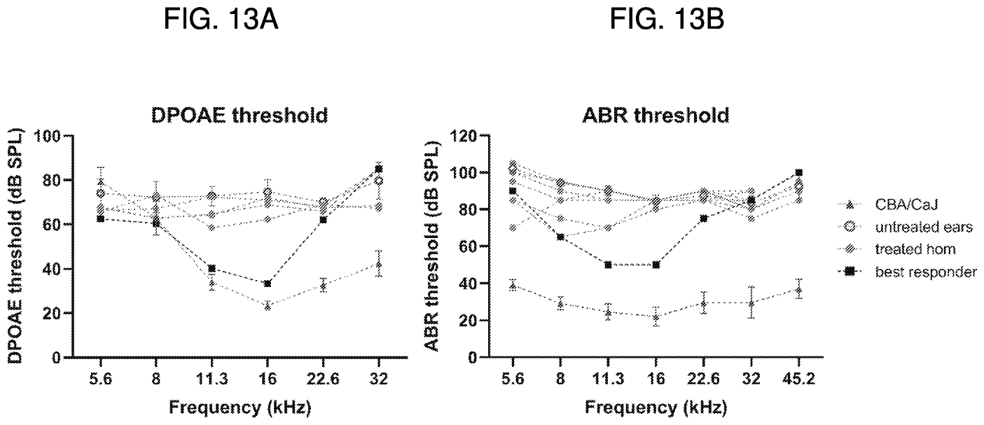

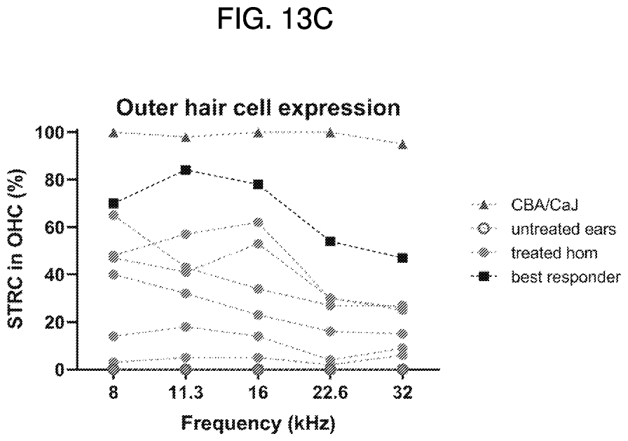

CRISPR-Cas9 technology was used to generate stereocilin deficient mice in the CBA/CaJ background strain by creating a frameshift at base pair position 232 of STRC. Wild type animals of the CBA/CaJ background strain showed distinct stereocilin antibody staining at the tips of the outer hair cell (OHC) stereocilia (FIG. 12A, bottom panel), while 232 bp Strc-KO animals lacked the signal for the antibody (FIG. 12B, bottom panel). Murine STRC was encapsulated in dual Anc80 vectors, where the first vector carried a CMV promoter and nucleotides 1-3200 of the murine STRC cDNA the second carried amino acids 2201-5430, creating a 1000 bp overlap between the two halves of the full-length cDNA. After delivery of both vectors at concentration of 1 E10 vg/ear into the cochlea of early postnatal 232 bp Strc-KO mice via their posterior semicircular canal, de-novo stereocilin protein expression could be observed at the tips of the OHC and in the body of inner hair cells of the organ of Corti in treated 232 bp Strc-KO mice (FIG. 12C, bottom panel). Distortion product otoacoustic emissions (DPOAE) were evaluated as a direct readout of OHC function and auditory brainstem response (ABR) was measured as a measure of an intact ascending auditory pathway four weeks after treatment with Anc80-CMV-mStrc (overlap). Untreated contralateral ears in 232 bp Strc-KO animals (“untreated ears”) showed near absent DPOAEs and highly elevated ABR thresholds indicative of loss of OHC function (FIGS. 13A-13B, open circles), while treated 232 bp Strc-KO animals (“treated hom”) showed recovery of hearing thresholds (FIGS. 13A-13B, filled circles). The best responder (FIGS. 13A-13B, black squares) of the treated 232 bp Strc-KO animals showed close to wild type (“CBA/CaJ”) (FIGS. 13A-13B, triangles) hearing thresholds. A high fraction of OHCs of 232 bp Strc-KO mice expressing stereocilin after treatment with AAV-Anc80-CMV-mStrc was found to promote hearing recovery (FIG. 13C).

### Example 4. A Two-Vector Split Intein System Reconstituted Full-Length Stereocilin In Vitro

To generate experimental plasmids, DNA encoding amino acids 1-746 of stereocilin (“N-Strc”) was genetically fused with DNA encoding the Npu N-intein fragment (SEQ ID NO: 40, which encodes the Npu N-intein of SEQ ID NO: 26) and cloned into a plasmid containing the constitutively active CMV promoter to generate CMV.N-Strc-N-Npu. DNA encoding amino acids 747-1809 of stereocilin (“C-Strc”) was genetically fused downstream of DNA encoding the Npu C-intein fragment (SEQ ID NO: 41, which encodes the Npu C-intein of SEQ ID NO: 27) and cloned into a plasmid containing the CMV promoter to produce CMV.C-Npu-C-Strc. As a control, the full-length stereocilin coding sequence (“FL-Strc”) was also cloned into a CMV plasmid to generate CMV.FL-Strc. CMV.GFP was used as a negative control.

HEK293T cells were transfected with either control plasmids or a combination of N-Strc and C-Strc plasmids using the Lipofectamine 3000 kit (Life Technologies) and were incubated under standard cell culture conditions for three days. Cell cultures were rinsed with PBS and cells were lysed to extract protein. Protein lysate concentrations were measured using the BCA assay, and a constant mass of protein was loaded for Western blotting using antibodies against beta actin and stereocilin. Densitometry measurements of the protein band intensities was used to determine the relative amount of full-length stereocilin from the sample.

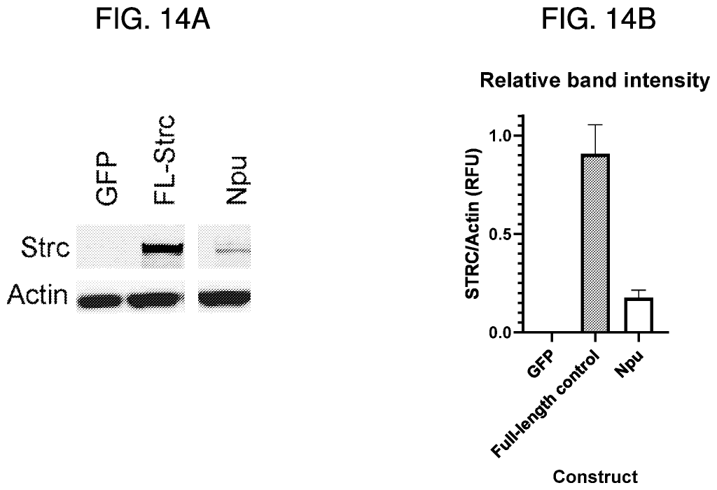

As shown in FIGS. 14A-14B, the tested intein designs produced a full-length stereocilin band.

### Example 5. Evaluation of Stereocilin Protein Expression Using Different Dual Vector Systems

AAV-DJ-CMV-mSTRC dual hybrid vectors were generated with different STRC split sites. The 5′ and 3′ vectors were separated at nucleotide position 1800, 2247, 2310, 2421, or 2588 (the 5′ vector contained nucleotides 1-1800, 1-2247, 1-2310, 1-2421, or 1-2588 of murine STRC and the 3′ vector contained the remaining nucleotides of the murine STRC coding sequence, e.g., nucleotides 1801-5430, 2248-5430, 2311-5430, 2422-5430, or 2589-5430). The recombinogenic region employed in the dual hybrid vectors was an AP gene fragment. An overlapping dual vector system was also tested in which the 5′ and 3′ vector contained a common 1000 nucleotides of murine stereocilin (the 5′ vector carried nucleotides 79-3278 of NM_080459 (corresponding to nucleotides 1-3200 of SEQ ID NO: 6) and the 3′ vector carried nucleotides 2279-5508 (corresponding to nucleotides 2201-5430 of SEQ ID NO: 6)).

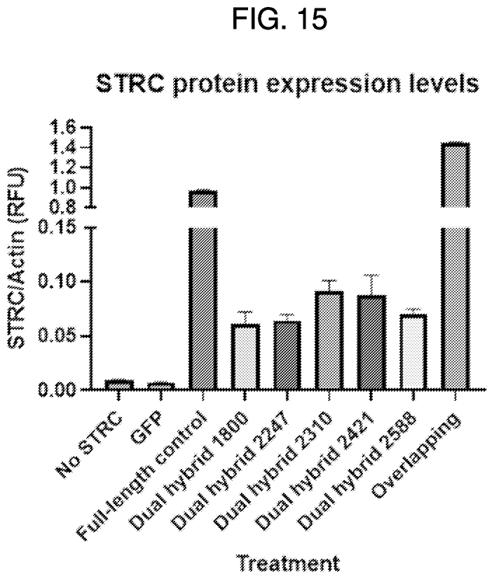

HEK293T cells were seeded into tissue culture plates. After cells adhered, AAV was added to the culture at a multiplicity of infection (MOI) of 1×10\hat{ }7 vector genomes per cell with the 5′ and 3′ vectors added at a 1:1 ratio. GFP and full-length STRC controls were treated with Lipofectamine 3000 containing plasmid DNA encoding the transgene instead of with AAV. Treated cells were incubated for three days under standard cell culture conditions, then cells were lysed and total protein was collected. 15 μg of lysate for each sample was loaded onto a 3-8% Tris-acetate/SDS gel and a standard Western blot was performed. Blotted membranes were treated with antibodies to specifically detect stereocilin protein or Actin. To obtain semi-quantitative measures of expression levels, densitometry of the detected bands was performed and the ratio of signal intensity between stereocilin and Actin detection was used to approximate stereocilin protein expression level (FIG. 15).

### Example 6. STRC Promoter-Driven GFP Expression in Murine Neonatal Cochlear Explants

To test the specificity of the STRC promoters disclosed herein in neonatal cochlear hair cells, we dissected sensory epithelia from P0-P2 mice and plated one or two to a dish on Matrigel-treated MatTek 35 mm dishes with a #0 10 mm coverslip. 150-200 μL of DMEM+10% FBS+10 μg/mL ciprofloxacin was added to each dish. After a one-hour incubation at 37° C./5% CO₂, an AAV vector expressing a nuclear directed eGFP-H2B fusion under control of various STRC promoters disclosed herein (see Table 6 below for details) was added at a dose of 1×10¹¹ vg to each dish. The explants were then incubated at 37° C./5% CO₂ for two days. After two days, the media+virus was removed and replaced with fresh media without virus. The explants were then incubated for an additional three days and fixed with 4% paraformaldehyde (PFA) at room temperature for 20 minutes.

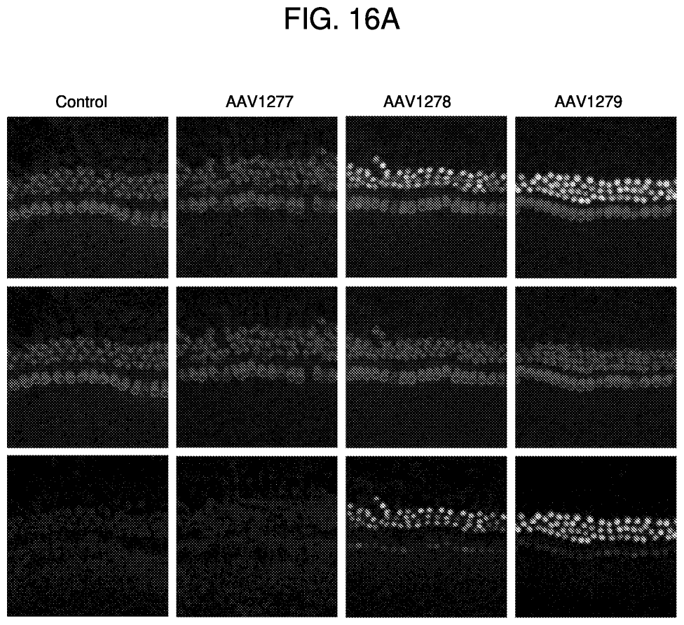

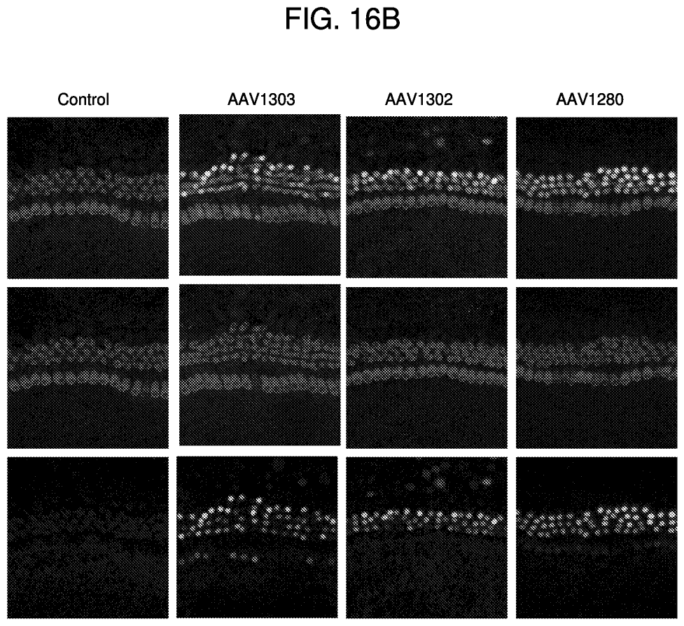

After incubation with PFA, the explants were washed 3× with PBS, then incubated in 10% normal donkey serum (NDS) in PBS for 20 minutes. The NDS was removed, and the explants were incubated with a primary antibody against Myo7a (a cochlear hair cell marker), diluted 1:1000 in PBS, overnight at 4° C. The following day, the explants were washed 3× with PBS, then incubated with a fluorescently labelled secondary antibody, diluted 1:1000 in PBS, for 2-3 hours at room temperature. After incubating in secondary antibodies, the explants were washed 5× with PBS and mounted onto microscope slides using Fluoromount mounting medium. Slides were then imaged using a Zeiss Upright Apotome light microscope for 20× images and a Zeiss LSM 880 confocal microscope for 40× images. Representative examples of the 40× images are shown in FIGS. 16A-16B.

TABLE 6
AAV vector constructs used to infect
neonatal murine cochlear explants

| | | | Dose | |
|---|---|---|---|---|
| Vector | Promoter | Titer | (vg) | N |
| AAV1303 | hStrc (SEQ ID NO: 1) | 1.24e14 | 1e11 | 3 |
| AAV1302 | mStrc nt 35-530 of SEQ ID NO: 2 | 2.28e14 | 1e11 | 3 |
| AAV1277 | mStrc nt 460-537 of SEQ ID NO: 2 | 2.97e13 | 1e11 | 3 |
| AAV1278 | mStrc nt 252-537 of SEQ ID NO: 2 | 2.73e13 | 1e11 | 3 |
| AAV1279 | mStrc nt 120-537 of SEQ ID NO: 2 | 2.90e13 | 1e11 | 3 |
| AAV1280 | mStrc (SEQ ID NO: 2) | 2.86e13 | 1e11 | 3 |

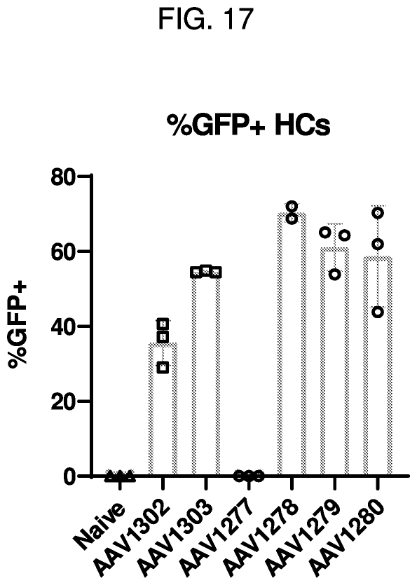

Quantification of cells expressing GFP following infection was done using Imaris software to analyze the 20× images of the whole cochlea acquired with the Upright Apotome. To quantify the number of total hair cells, Myo7a⁺ cells were quantified using the spots feature of the Imaris software. These cells were then filtered by intensity of the GFP channel. The threshold of the filter was manually set so that only spots with Myo7a and GFP were selected. This provided the total number of GFP+ hair cells in each sample. The results are shown in FIG. 17.

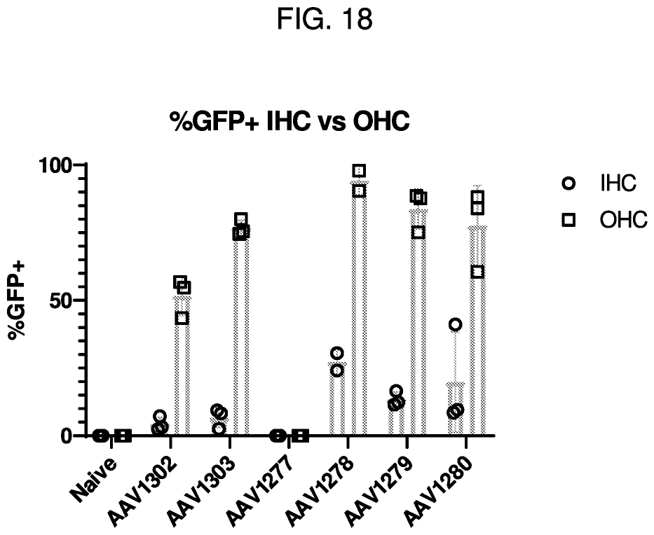

To quantify the number of inner hair cells and outer hair cells, Myo7a+ spots representing inner and outer hair cells were manually separated. These spots were then filtered by intensity of the GFP signal to separately quantify GFP+ inner and outer hair cells. The results are shown in FIG. 18.

### Example 7. Administration of a Composition Containing a Nucleic Acid Vector Containing an STRC Promoter to a Subject with Sensorineural Hearing Loss

According to the methods disclosed herein, a physician of skill in the art can treat a patient, such as a human patient, with hearing loss (e.g., sensorineural hearing loss) so as to improve or restore hearing. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S vector) containing an STRC promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, such as nucleotides 35-530 of SEQ ID NO: 2, or a functional portion of SEQ ID NO: 48 containing nucleotides 280-560 of SEQ ID NO: 48) operably linked to a polynucleotide encoding a desired expression product (e.g., a transgene encoding a protein that plays a role in hair cell development, regeneration, cell fate specification, maintenance, function, or survival, or a wild-type version of a gene associated with hearing loss that is mutated in the subject). The composition containing the AAV vector may be administered to the patient, for example, by local administration to the inner ear (e.g., injection into the perilymph or through the round window membrane), to treat sensorineural hearing loss.

Following administration of the composition to a patient, a practitioner of skill in the art can monitor the patient's improvement in response to the therapy by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### Example 8. Administration of a Composition Containing a Nucleic Acid Vector Containing an STRC Promoter to a Subject with Vestibular Dysfunction

According to the methods disclosed herein, a physician of skill in the art can treat a patient, such as a human patient, with vestibular dysfunction (e.g., bilateral vestibulopathy) so as to improve or restore vestibular function (e.g., improve balance or reduce falls). To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S vector) containing an STRC promoter described herein (e.g., a polynucleotide having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, such as nucleotides 35-530 of SEQ ID NO: 2, or a functional portion of SEQ ID NO: 48 containing nucleotides 280-560 of SEQ ID NO: 48) operably linked to a polynucleotide encoding a desired expression product (e.g., a transgene encoding a protein that plays a role in hair cell development, regeneration, cell fate specification, maintenance, function, or survival, or a wild-type version of a gene associated with vestibular dysfunction that is mutated in the subject). The composition containing the AAV vector may be administered to the patient, for example, by local administration to the inner ear (e.g., injection into a semicircular canal, such as the horizontal canal), to treat vestibular dysfunction.

Following administration of the composition to a patient, a practitioner of skill in the art can monitor the patient's improvement in response to the therapy by a variety of methods. For example, a physician can monitor the patient's vestibular function by performing standard tests such as electronystagmography, video nystagmography, rotation tests, tests of the VOR, vestibular evoked myogenic potential, or computerized dynamic posturography. A finding that the patient exhibits improved vestibular function in one or more of the tests following administration of the composition compared to test results obtained prior to administration of the composition indicates that the patient is responding favorably to the treatment. Subsequent doses can be determined and administered as needed.

### Example 9. Administration of a Composition Containing a Two-Vector System Containing a STRC Promoter Operably Linked to a Stereocilin Coding Sequence to a Subject with Sensorineural Hearing Loss

According to the methods disclosed herein, a physician of skill in the art can treat a patient, such as a human patient, with sensorineural hearing loss (e.g., sensorineural hearing loss associated with a mutation in STRC, such as DFNB16) so as to improve or restore hearing. To this end, a physician of skill in the art can administer to the human patient a composition containing an two-vector nucleic acid expression system, such as system that utilizes two AAV vectors (e.g., AAV1, AAV2, AAV2quad(Y-F), AAV6, AAV8, AAV9, Anc80, Anc80L65, DJ/9, 7m8, or PHP.B vectors) that collectively include a STRC promoter (e.g., a polynucleotide having at least 85% sequence identity (e.g., 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, such as nucleotides 35-530 of SEQ ID NO: 2, or a functional portion of SEQ ID NO: 48 containing nucleotides 280-560 of SEQ ID NO: 48) operably linked to a STRC transgene.

The two-vector system may be an overlapping dual vector system containing a first and second AAV vector. The overlapping dual vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3) and a second AAV vector that includes a polynucleotide encoding a C-terminal portion of the stereocilin protein, in which the 3′ end of the stereocilin coding sequence in the first vector overlaps with the 5′ end of the stereocilin coding sequence in the second vector. In another example, the two-vector system may be a trans-splicing dual vector system containing a first and a second AAV vector. The trans-splicing dual vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3) and a splice donor signal sequence 3′ of the polynucleotide and a second AAV vector that includes a splice acceptor signal sequence 5′ of a polynucleotide encoding a C-terminal portion of the stereocilin protein. In another example, the two-vector system be a dual hybrid vector system containing a first and second AAV vector. The dual hybrid vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3), a splice donor signal sequence 3′ of the polynucleotide, and a first recombinogenic region 3′ of the splice donor signal sequence, and a second AAV vector that includes a second recombinogenic region, a splice acceptor signal sequence 3′ of the recombinogenic region, and a polynucleotide encoding a C-terminal portion of the stereocilin protein 3′ of the splice acceptor signal sequence. In yet another example, the two-vector system may be a split intein trans-splicing system that includes a first AAV vector and a second AAV vector. The split intein trans-splicing two-vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3) and a polynucleotide encoding an N-terminal intein (N-intein) 3′ thereto, and a second AAV vector that includes the STRC promoter operably linked to a polynucleotide encoding a C-terminal intein (C-intein) and a polynucleotide encoding a C-terminal portion of the stereocilin protein 3′ thereto. The aforementioned two-vector systems may additionally include regulatory sequences such as, e.g., enhancers, poly(A) sequences, and STRC untranslated regions (UTRs, e.g., a 5′ UTR and/or a 3′ UTR) that are not part of the promoters described herein.

The composition containing the AAV vectors may be administered to the patient, for example, by local administration to the inner ear (e.g., injection into the perilymph or through the round window membrane), to treat sensorineural hearing loss.

Following administration of the composition to a patient, a practitioner of skill in the art can monitor the expression of the therapeutic protein encoded by the transgene, and the patient's improvement in response to the therapy, by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

### Example 10. Administration of a Composition Containing a Two-Vector System Containing a STRC Promoter Operably Linked to a Stereocilin Coding Sequence to a Subject with Vestibular Dysfunction

According to the methods disclosed herein, a physician of skill in the art can treat a patient, such as a human patient, with a vestibular dysfunction (e.g., a vestibular dysfunction associated with a mutation in STRC, such as, e.g., vertigo, dizziness, imbalance, bilateral vestibulopathy, oscillopsia, or a balance disorder) so as to improve vestibular function. To this end, a physician of skill in the art can administer to the human patient a composition containing an two-vector nucleic acid expression system, such as system that utilizes two AAV vectors (e.g., AAV1, AAV2, AAV2quad(Y-F), AAV6, AAV8, AAV9, Anc80, Anc80L65, DJ/9, 7m8, or PHP.B vectors) that collectively include a STRC promoter (e.g., a polynucleotide having at least 85% sequence identity (e.g., 86%, 87%, 88%, 89%, 90%, 91%, 92%, 93%, 94%, 95%, 96%, 97%, 98%, 99%, or more, sequence identity) to SEQ ID NO: 1, SEQ ID NO: 2, SEQ ID NO: 48, a functional portion of SEQ ID NO: 2 containing nucleotides 252-537 or 35-530 of SEQ ID NO: 2, or a functional portion of SEQ ID NO: 48 containing nucleotides 280-560 of SEQ ID NO: 48) operably linked to a STRC transgene.

The two-vector system may be an overlapping dual vector system containing a first and second AAV vector. The overlapping dual vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3) and a second AAV vector that includes a polynucleotide encoding a C-terminal portion of the stereocilin protein, in which the 3′ end of the stereocilin coding sequence in the first vector overlaps with the 5′ end of the stereocilin coding sequence in the second vector. In another example, the two-vector system may be a trans-splicing dual vector system containing a first and a second AAV vector. The trans-splicing dual vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3) and a splice donor signal sequence 3′ of the polynucleotide and a second AAV vector that includes a splice acceptor signal sequence 5′ of a polynucleotide encoding a C-terminal portion of the stereocilin protein. In another example, the two-vector system be a dual hybrid vector system containing a first and second AAV vector. The dual hybrid vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3), a splice donor signal sequence 3′ of the polynucleotide, and a first recombinogenic region 3′ of the splice donor signal sequence, and a second AAV vector that includes a second recombinogenic region, a splice acceptor signal sequence 3′ of the recombinogenic region, and a polynucleotide encoding a C-terminal portion of the stereocilin protein 3′ of the splice acceptor signal sequence. In yet another example, the two-vector system may be a split intein trans-splicing system that includes a first AAV vector and a second AAV vector. The split intein trans-splicing two-vector system may include a first AAV vector that includes the STRC promoter operably linked to a polynucleotide encoding an N-terminal portion of a stereocilin protein (e.g., an N-terminal portion of SEQ ID NO: 3 or a variant thereof having at least 85% (e.g., at least 86%, 87%, 88%, 89%, 90%, 91%, 92%, 93%, 94%, 95%, 96%, 97%, 98%, 99%, or more) sequence identity to the amino acid sequence of SEQ ID NO: 3) and a polynucleotide encoding an N-terminal intein (N-intein) 3′ thereto, and a second AAV vector that includes the STRC promoter operably linked to a polynucleotide encoding a C-terminal intein (C-intein) and a polynucleotide encoding a C-terminal portion of the stereocilin protein 3′ thereto. The aforementioned two-vector systems may additionally include regulatory sequences such as, e.g., enhancers, poly(A) sequences, and STRC untranslated regions (UTRs, e.g., a 5′ UTR and/or a 3′ UTR) that are not part of the promoters described herein.

The composition containing the AAV vectors may be administered to the patient, for example, by local administration to the inner ear (e.g., injection to a semicircular canal, e.g., the horizontal canal), to treat vestibular dysfunction.

Following administration of the composition to a patient, a practitioner of skill in the art can monitor the expression of the therapeutic protein encoded by the transgene, and the patient's improvement in response to the therapy, by a variety of methods. For example, a physician can monitor the patient's vestibular function by performing standard tests, such as, e.g., eye movement testing (e.g., electronystagmogram (ENG) or videonystagmogram (VNG)), tests of the vestibulo-ocular reflex (VOR)(e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini et al., Eur J Phys Rehabil Med 46:239 (2010) following administration of the composition. A finding that the patient exhibits improved vestibular function in one or more of the tests following administration of the composition compared to vestibular function test results prior to administration of the composition indicates that the patient is responding favorably to the treatment. Subsequent doses can be determined and administered as needed.

Exemplary embodiments of the invention are described in the enumerated paragraphs below.
E1. A polynucleotide comprising a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof comprising nucleotides 280-560 of SEQ ID NO: 48 operably linked to a polynucleotide encoding a heterologous expression product.
E2. A polynucleotide comprising a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2, operably linked to a polynucleotide encoding a heterologous expression product.
E3. The polynucleotide of E1, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48.
E4. The polynucleotide of E1 or E3, wherein the STRC promoter has the sequence of SEQ ID NO: 48.
E5. The polynucleotide of E1, wherein the functional portion of SEQ ID NO: 48 comprises or consists of nucleotides 280-560 of SEQ ID NO: 48.
E6. The polynucleotide of E1, wherein the functional portion of SEQ ID NO: 48 comprises or consists of nucleotides 280-564 of SEQ ID NO: 48.
E7. The polynucleotide of E1, wherein the functional portion of SEQ ID NO: 48 comprises or consists of nucleotides 124-560 of SEQ ID NO: 48.
E8. The polynucleotide of E1, wherein the functional portion of SEQ ID NO: 48 comprises or consists of nucleotides 124-564 of SEQ ID NO: 48.
E9. The polynucleotide of E1, wherein the functional portion of SEQ ID NO: 48 comprises or consists of nucleotides 1-560 of SEQ ID NO: 48.
E10. The polynucleotide of E2, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E11. The polynucleotide of E2 or E10, wherein the STRC promoter consists of SEQ ID NO: 1.
E12. The polynucleotide of E2, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2.
E13. The polynucleotide of E2 or E12, wherein the functional portion of SEQ ID NO: 2 comprises or consists of nucleotides 252-537 of SEQ ID NO: 2.
E14. The polynucleotide of E2 or E12, wherein the functional portion of SEQ ID NO: 2 comprises or consists of nucleotides 120-537 of SEQ ID NO: 2.
E15. The polynucleotide of E2 or E12, wherein the functional portion of SEQ ID NO: 2 comprises or consists of nucleotides 35-530 of SEQ ID NO: 2.
E16. The polynucleotide of E2 or E12, wherein the STRC promoter consists of SEQ ID NO: 2.
E17. The polynucleotide of any one of E1-E16, wherein the heterologous expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E18. The polynucleotide of E17, wherein the protein is Actin Gamma 1 (ACTG1), Fascin Actin-Bundling Protein 2, Retinal (FSCN2), Radixin (RDX), POU Class 4 Homeobox 3 (POU4F3), TRIO and F-Actin Binding Protein (TRIOBP), Taperin (TPRN), Xin Actin Binding Repeat Containing 2 (XIRP2), Atonal BHLH Transcription Factor 1 (ATOH1), Growth Factor Independent 1 Transcriptional Repressor (GFI1), Cholinergic Receptor Nicotinic Alpha 9 Subunit (CHRNA9), Cholinergic Receptor Nicotinic Alpha 10 Subunit (CHRNA10), Calcium and Integrin Binding Family Member 3 (CIB3), Cadherin 23 (CDH23), Protocadherin 15 (PCDH15), Kinocilin (KNCN), Pejvakin (DFNB59), MKRN2 Opposite Strand (MKRN2OS), LIM Homeobox Protein 3 (LHX3), Transmembrane Channel Like 1 (TMC1), Myosin 15 (MYO15), Myosin 7A (MYO7A), Myosin 6 (MYO6), Myosin IIIA (MYO3A), Myosin IIIB (MYO3B), Glutaredoxin Domain Containing Cysteine-Rich Protein 1 (GRXCR1), Protein Tyrosine Phosphatase, Receptor Type Q (PTPRQ), Late Cornified Envelope 6A (LCE6A), Lipoxygenase Homology Domain-containing Protein 1 (LOXHD1), ADP-Ribosyltransferase 1 (ART1), ATPase Plasma Membrane Ca2+ Transporting 2 (ATP2B2), Calcium and Integrin Binding Family Member 2 (CIB2), Calcium Voltage-Gated Channel Auxiliary Subunit Alpha2delta 4 (CACNA2D4), Epidermal Growth Factor Receptor Pathway Substrate 8 (EPS8), EPS8 Like 2 (EPS8L2), Espin (ESPN), Espin Like (ESPNL), Peripherin 2 (PRPH2), Solute Carrier Family 8 Member A2 (SLC8A2), Zinc Finger CCHC-Type Containing Protein 12 (ZCCHC12), Leucine Rich Transmembrane and O-methyltransferase Domain Containing (LRTOMT2, LRTOMT1), USH1 Protein Network Component Harmonin (USH1C), Solute Carrier Family 26 Member 5 (SLC26A5), Piezo Type Mechanosensitive Ion Channel Component 2 (PIEZO2), Extracellular Leucine Rich Repeat and Fibronectin Type III Domain Containing 1 (ELFN1), Tetratricopeptide Repeat Protein 24 (TTC24), Dystrotelin (DYTN), Coiled-coil Glutamate Rich Protein 2 (CCER2), Leucine-rich Repeat and Transmembrane Domain-containing protein 2 (LRTM2), Potassium Voltage-Gated Channel Subfamily A Member 10 (KCNA10), Clarin 1 (CLRN1), Clarin 2 (CLRN2), SKI Family Transcriptional Corepressor 1 (SKOR1), Tctexl Domain Containing Protein 1 (TCTEX1 D1), Fc Receptor Like B (FCRLB), Glutaredoxin Domain Containing Cysteine-Rich Protein 2 (GRXCR2), Serpin Family E Member 3 (SERPINE3), Nescient Helix-loop Helix 1 (NHLH1), Heat Shock Protein 70 (HSP70), Heat Shock Protein 90 (HSP90), Activating Transcription Factor 6 (ATF6), Eukaryotic Translation Initiation Factor 2 Alpha Kinase 3 (PERK), Serine/Threonine-Protein Kinase/Endoribonuclease IRE1 (IRE1), Whirlin (WHRN), Oncomodulin (OCM), LIM Homeobox 1 (Isl1), Transmembrane and Tetratricopeptide Repeat Containing 4 (TMTC4), Binding Immunoglobulin Protein (BIP), or Potassium Voltage-Gated Channel Subfamily Q Member 4 (KCNQ4).
E19. A nucleic acid vector comprising the polynucleotide of any one of E1-E18.
E20. A nucleic acid vector comprising a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof comprising nucleotides 280-560 of SEQ ID NO: 48.
E21. A nucleic acid vector comprising a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2.
E22. The nucleic acid vector of E20, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48.
E23. The nucleic acid vector of E20 or E22, wherein the STRC promoter has the sequence of SEQ ID NO: 48.
E24. The nucleic acid vector of E20, wherein the functional portion of SEQ ID NO: 48 comprises or consists of nucleotides 280-560 of SEQ ID NO: 48.
E25. The nucleic acid vector of E20, wherein the functional portion of SEQ ID NO: 48 comprises or consists of nucleotides 280-564 of SEQ ID NO: 48.
E26. The nucleic acid vector of E20, wherein the functional portion of SEQ ID NO: 48 comprises or consists of nucleotides 124-560 of SEQ ID NO: 48.
E27. The nucleic acid vector of E20, wherein the functional portion of SEQ ID NO: 48 comprises or consists of nucleotides 124-564 of SEQ ID NO: 48.
E28. The nucleic acid vector of E20, wherein the functional portion of SEQ ID NO: 48 comprises or consists of nucleotides 1-560 of SEQ ID NO: 48.
E29. The nucleic acid vector of E21, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E30. The nucleic acid vector of E21 or E29, wherein the STRC promoter consists of SEQ ID NO: 1.
E31. The nucleic acid vector of E21, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2.
E32. The nucleic acid vector of E21 or E31, wherein the functional portion of SEQ ID NO: 2 comprises or consists of nucleotides 252-537 of SEQ ID NO: 2.
E33. The nucleic acid vector of E21 or E31, wherein the functional portion of SEQ ID NO: 2 comprises or consists of nucleotides 120-537 of SEQ ID NO: 2.
E34. The nucleic acid vector of E21 or E31, wherein the functional portion of SEQ ID NO: 2 comprises or consists of nucleotides 35-530 of SEQ ID NO: 2.
E35. The nucleic acid vector of E20 or E31, wherein the STRC promoter consists of SEQ ID NO: 2.
E36. The nucleic acid vector of any one of E20-E35, wherein the STRC promoter is operably linked to a polynucleotide encoding a heterologous expression product.
E37. The nucleic acid vector of E36, wherein the heterologous expression product is a protein, a short hairpin RNA (shRNA), an antisense oligonucleotide (ASO), a component of a gene editing system (e.g., a nuclease, such as a CRISPR Associated Protein 9 (Cas9), Transcription Activator-Like Effector Nuclease (TALEN), or Zinc Finger Nuclease (ZFN), or a guide RNA (gRNA)), or a microRNA.
E38. The nucleic acid vector of E37, wherein the protein is ACTG1, FSCN2, RDX, POU4F3, TRIOBP, TPRN, XIRP2, ATOH1, GFI1, CHRNA9, CHRNA10, CIB3, CDH23, PCDH15, KNCN, DFNB59, MKRN2OS, LHX3, TMC1, MYO15, MYO7A, MYO6, MYO3A, MYO3B, GRXCR1, PTPRQ, LCE6A, LOXHD1, ART1, ATP2B2, CIB2, CACNA2D4, EPS8, EPS8L2, ESPN, ESPNL, PRPH2, SLC8A2, ZCCHC12, LRTOMT2, LRTOMT1, USH1C, SLC26A5, PIEZO2, ELFN1, TTC24, DYTN, CCER2, LRTM2, KCNA10, CLRN1, CLRN2, SKOR1, TCTEX1D1, FCRLB, GRXCR2, SERPINE3, NHLH1, HSP70, HSP90, ATF6, PERK, IRE1, WHRN, OCM, Isl1, TMTC4, BIP, or KCNQ4.
E39. The nucleic acid vector of any one of E20-E35, wherein the STRC promoter is operably linked to a polynucleotide encoding an N-terminal portion of a stereocilin protein that does not encode a full-length stereocilin protein.
E40. The nucleic acid vector of E39, wherein the nucleic acid vector is a first nucleic acid vector in a two-vector system that further comprises a second nucleic acid vector comprising a polynucleotide encoding a C-terminal portion of a stereocilin protein that does not encode a full-length stereocilin protein.
E41. The nucleic acid vector of any one of E19-E40, wherein the nucleic acid vector is a viral vector, plasmid, cosmid, or artificial chromosome.
E42. The nucleic acid vector of E41, wherein the nucleic acid vector is a viral vector selected from the group consisting of an adeno-associated virus (AAV), an adenovirus, and a lentivirus.
E43. The nucleic acid vector of E42, wherein the viral vector is an AAV vector.
E44. The nucleic acid vector of E43, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid.
E45. A composition comprising the nucleic acid vector of any one of E19-E44.
E46. The composition of E45, further comprising a pharmaceutically acceptable carrier, diluent, or excipient.
E47. A cell comprising the polynucleotide of any one of E1-E18 or the nucleic acid vector of any one of E19-E44.
E48. The cell of E47, wherein the cell is a hair cell.
E49. The cell of E48, wherein the hair cell is a mammalian hair cell.
E50. The cell of E49, wherein the mammalian hair cell is a human hair cell.
E51. The cell of any one of E48-E50, wherein the hair cell is a cochlear hair cell.
E52. The cell of E51, wherein the cochlear hair cell is an outer hair cell.
E53. The cell of E51, wherein the cochlear hair cell is an inner hair cell.
E54. The cell of any one of E48-E50, wherein the hair cell is a vestibular hair cell.
E55. The cell of E54, wherein the vestibular hair cell is a type II vestibular hair cell.
E56. The cell of E54, wherein the vestibular hair cell is a type I vestibular hair cell.
E57. A method of expressing a heterologous expression product in a hair cell, comprising contacting the hair cell with the nucleic acid vector of any one of E19-E44 or the composition of E45 or E46.
E58. The method of E57, wherein the expression product is specifically expressed in hair cells.
E59. A method of treating a subject having or at risk of developing hearing loss (e.g., sensorineural hearing loss, auditory neuropathy, or deafness), comprising administering to an inner ear of the subject an effective amount of the nucleic acid vector of any one of E19-E44 or the composition of E45 or E46.
E60. A method of treating a subject having or at risk of developing tinnitus, comprising administering to an inner ear of the subject an effective amount of the nucleic acid vector of any one of E19-E44 or the composition of E45 or E46.
E61. A method of treating a subject having or at risk of developing vestibular dysfunction, comprising administering to an inner ear of the subject an effective amount of the nucleic acid vector of any one of E19-E44 or the composition of E45 or E46.
E62. A method of treating a subject having or at risk of developing bilateral vestibulopathy, comprising administering to an inner ear of the subject an effective amount of the nucleic acid vector of any one of E19-E44 or the composition of E45 or E46.
E63. The method of E62, wherein the bilateral vestibulopathy is ototoxic drug-induced bilateral vestibulopathy.
E64. A method of treating a subject having or at risk of developing oscillopsia, comprising administering to an inner ear of the subject an effective amount of the nucleic acid vector of any one of E19-E44 or the composition of E45 or E46.
E65. The method of E64, wherein the oscillopsia is ototoxic drug-induced oscillopsia.
E66. A method of treating a subject having or at risk of developing a balance disorder, comprising administering to an inner ear of the subject an effective amount of the nucleic acid vector of any one of E19-E44 or the composition of E45 or E46.
E67. A method of inducing or increasing hair cell regeneration in a subject in need thereof, comprising administering to an inner ear of the subject an effective amount of the nucleic acid vector of any one of E19-E44 or the composition of E45 or E46.
E68. A method of increasing hair cell maintenance in a subject in need thereof, comprising administering to an inner ear of the subject an effective amount of the nucleic acid vector of any one of E19-E44 or the composition of E45 or E46.
E69. A method of increasing hair cell survival in a subject in need thereof, comprising administering to an inner ear of the subject an effective amount of the nucleic acid vector of any one of E19-E44 or the composition of E45 or E46.
E70. A method of inducing or increasing hair cell maturation in a subject in need thereof, comprising administering to an inner ear of the subject an effective amount of the nucleic acid vector of any one of E19-E44 or the composition of E45 or E46.
E71. A method of preventing or reducing ototoxic drug-induced hair cell damage or death in a subject in need thereof, comprising administering to an inner ear of the subject an effective amount of the nucleic acid vector of any one of E19-E44 or the composition of E45 or E46.
E72. A method of preventing or reducing hair cell damage or death in a subject in need thereof, comprising administering to an inner ear of the subject an effective amount of the nucleic acid vector of any one of E19-E44 or the composition of E45 or E46.
E73. The method of any one of E57, E58, and E67-E72, wherein the hair cell is a mammalian hair cell.
E74. The method of E73, wherein the mammalian hair cell is a human hair cell.
E75. The method of any one of E57, E58, and E67-E74, wherein the hair cell is a cochlear hair cell.
E76. The method of E75, wherein the cochlear hair cell is an outer hair cell.
E77. The method of E75, wherein the cochlear hair cell is an inner hair cell.
E78. The method of any one of E57, E58, and E67-E74, wherein the hair cell is a vestibular hair cell.
E79. The method of E78, wherein the vestibular hair cell is a type II vestibular hair cell.
E80. The method of E78, wherein the vestibular hair cell is a type I vestibular hair cell.
E81. The method of any one of E67-E77, wherein the subject has or is at risk of developing hearing loss (e.g., sensorineural hearing loss).
E82. The method of E59 or E81, wherein the hearing loss is genetic hearing loss.
E83. The method of E82, wherein the genetic hearing loss is autosomal dominant hearing loss, autosomal recessive hearing loss, or X-linked hearing loss.
E84. The method of E59 or E81, wherein the hearing loss is acquired hearing loss.
E85. The method of E84, wherein the acquired hearing loss is noise-induced hearing loss, age-related hearing loss, disease or infection-related hearing loss, head trauma-related hearing loss, or ototoxic drug-induced hearing loss.
E86. The method of any one of E67-E72, wherein the subject has or is at risk of developing vestibular dysfunction.
E87. The method of E61 or E86, wherein the vestibular dysfunction comprises vertigo, dizziness, imbalance, bilateral vestibulopathy, oscillopsia, or a balance disorder.
E88. The method of any one of E61, E86, and E87, wherein the vestibular dysfunction is age-related vestibular dysfunction, head trauma-related vestibular dysfunction, disease or infection-related vestibular dysfunction, or ototoxic drug-induced vestibular dysfunction.
E89. The method of any one of E61 and E86-E88, wherein the vestibular dysfunction is associated with a genetic mutation.
E90. The method of any one of E61, E86, and E87, wherein the vestibular dysfunction is idiopathic vestibular dysfunction.
E91. The method of any one of E63, E65, E71, E85, and E88, wherein the ototoxic drug is an aminoglycoside, an antineoplastic drug, ethacrynic acid, furosemide, a salicylate, or quinine.
E92. The method of any one of E59, E60, E67-E77, and E81-E85, wherein the method further comprises evaluating the hearing of the subject prior to administering the nucleic acid vector or composition.
E93. The method of any one of E59, E60, E67-E77, E81-E85, and E92, wherein the method further comprises evaluating the hearing of the subject after administering the nucleic acid vector or composition.
E94. The method of any one of E61-E74, E78-E80, and E86-E93, wherein the method further comprises evaluating the vestibular function of the subject prior to administering the nucleic acid vector or composition.
E95. The method of any one of E61-E74, E78-E80, and E86-E94, wherein the method further comprises evaluating the vestibular function of the subject after administering the nucleic acid vector or composition.
E96. The method of any one of E59-E95, wherein the nucleic acid vector or composition is locally administered.
E97. The method of E96, wherein the nucleic acid vector or composition is administered to the inner ear.
E98. The method of E96, wherein the nucleic acid vector or composition is administered to the middle ear.
E99. The method of E96, wherein the nucleic acid vector or composition is administered to a semicircular canal.
E100. The method of E96, wherein the nucleic acid vector or composition is administered transtympanically or intratympanically.
E101. The method of E96, wherein the nucleic acid vector or composition is administered into the perilymph.
E102. The method of E96, wherein the nucleic acid vector or composition is administered into the endolymph.
E103. The method of E96, wherein the nucleic acid vector or composition is administered to or through the oval window.
E104. The method of E96, wherein the nucleic acid vector or composition is administered to or through the round window.
E105. The method of any one of E59-E104, wherein the nucleic acid vector or composition is administered in an amount sufficient to prevent or reduce vestibular dysfunction, delay the development of vestibular dysfunction, slow the progression of vestibular dysfunction, improve vestibular function, prevent or reduce hearing loss, prevent or reduce tinnitus, delay the development of hearing loss, slow the progression of hearing loss, improve hearing, increase hair cell numbers, increase hair cell maturation, increase hair cell regeneration, improve hair cell function, prevent or reduce hair cell damage, prevent or reduce hair cell death, or promote or increase hair cell survival.
E106. The method of E57 or E58, wherein the contacting is in vivo (in a subject).
E107. The method of any one of E59-E106, wherein the subject is a human subject.
E108. A two-vector system comprising:
   a) a first nucleic acid vector comprising a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof comprising nucleotides 280-560 of SEQ ID NO: 48 operably linked to a first polynucleotide encoding an N-terminal portion of a stereocilin protein; and
   b) a second nucleic acid vector comprising a second polynucleotide encoding a C-terminal portion of a stereocilin protein.
E109. A two-vector system comprising:
   a) a first nucleic acid vector comprising a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2, operably linked to a first polynucleotide encoding an N-terminal portion of a stereocilin protein; and
   b) a second nucleic acid vector comprising a second polynucleotide encoding a C-terminal portion of a stereocilin protein.
E110. The two-vector system of E108, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48.
E111. The two-vector system of E108 or E110, wherein the STRC promoter has the sequence of SEQ ID NO: 48.
E112. The two-vector system of E108, wherein the functional portion of SEQ ID NO: 48 comprises or consists of nucleotides 280-560 of SEQ ID NO: 48.
E113. The two-vector system of E108, wherein the functional portion of SEQ ID NO: 48 comprises or consists of nucleotides 280-564 of SEQ ID NO: 48.
E114. The two-vector system of E108, wherein the functional portion of SEQ ID NO: 48 comprises or consists of nucleotides 124-560 of SEQ ID NO: 48.
E115. The two-vector system of E108, wherein the functional portion of SEQ ID NO: 48 comprises or consists of nucleotides 124-564 of SEQ ID NO: 48.
E116. The two-vector system of E108, wherein the functional portion of SEQ ID NO: 48 comprises or consists of nucleotides 1-560 of SEQ ID NO: 48.
E117. The two-vector system of E109, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E118. The two-vector system of E109 or E117, wherein the STRC promoter consists of SEQ ID NO: 1.
E119. The two-vector system of E109, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2.
E120. The two-vector system of E109 or E119, wherein the functional portion of SEQ ID NO: 2 comprises or consists of nucleotides 252-537 of SEQ ID NO: 2.
E121. The two-vector system of E109 or E119, wherein the functional portion of SEQ ID NO: 2 comprises or consists of nucleotides 120-537 of SEQ ID NO: 2.
E122. The two-vector system of E109 or E119, wherein the functional portion of SEQ ID NO: 2 comprises or consists of nucleotides 35-530 of SEQ ID NO: 2.
E123. The two-vector system of E109 or E119, wherein the STRC promoter consists of SEQ ID NO: 2.
E124. The two-vector system of any one of E108-E123, wherein the first polynucleotide partially overlaps with the second polynucleotide.
E125. The two-vector system of any one of E108-E124, wherein the first polynucleotide and the second polynucleotide have a region of overlap having a length of at least 200 bases (b).
E126. The two-vector system of any one of E108-E125, wherein when introduced into a mammalian cell, the first and second nucleic acid vectors undergo homologous recombination to form a recombined polynucleotide that encodes a full-length stereocilin protein.
E127. The two-vector system of any one of E108-E123, wherein the first nucleic acid vector comprises a splice donor signal sequence positioned 3′ of the first polynucleotide and the second nucleic acid vector comprises a splice acceptor signal sequence positioned 5′ of the second polynucleotide.
E128. The two-vector system of any one of E108-E123, wherein the first nucleic acid vector comprises a splice donor signal sequence positioned 3′ of the first polynucleotide and a first recombinogenic region positioned 3′ of the splice donor signal sequence and the second nucleic acid vector comprises a second recombinogenic region, a splice acceptor signal sequence positioned 3′ of the recombinogenic region, and the second polynucleotide positioned 3′ of the splice acceptor signal sequence.
E129. The two-vector system of any one of E108-E123, E127, and E128, wherein the first and second polynucleotides do not overlap.
E130. The two-vector system of E128 or E129, wherein the first nucleic acid vector further comprises a degradation signal sequence positioned 3′ of the recombinogenic region; and wherein the second nucleic acid vector further comprises a degradation signal sequence positioned between the recombinogenic region and the splice acceptor signal sequence.
E131. The two-vector system of any one of E108-E123, wherein the second nucleic acid vector further comprises a STRC promoter having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48 or a functional portion thereof comprising nucleotides 280-560 of SEQ ID NO: 48 operably linked to the second polynucleotide, wherein the STRC promoter is positioned 5′ of the second polynucleotide.
E132. The two-vector system of any one of E108-E123, wherein the second nucleic acid vector further comprises a STRC promoter having: (i) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1; or (ii) at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2, operably linked to the second polynucleotide, wherein the STRC promoter is positioned 5′ of the second polynucleotide.
E133. The two-vector system of E131, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 48.
E134. The two-vector system of E131 or E133, wherein the STRC promoter has the sequence of SEQ ID NO: 48.
E135. The two-vector system of E131, wherein the functional portion of SEQ ID NO: 48 comprises or consists of nucleotides 280-560 of SEQ ID NO: 48.
E136. The two-vector system of E131, wherein the functional portion of SEQ ID NO: 48 comprises or consists of nucleotides 280-564 of SEQ ID NO: 48.
E137. The two-vector system of E131, wherein the functional portion of SEQ ID NO: 48 comprises or consists of nucleotides 124-560 of SEQ ID NO: 48.
E138. The two-vector system of E131, wherein the functional portion of SEQ ID NO: 48 comprises or consists of nucleotides 124-564 of SEQ ID NO: 48.
E139. The two-vector system of E131, wherein the functional portion of SEQ ID NO: 48 comprises or consists of nucleotides 1-560 of SEQ ID NO: 48.
E140. The two-vector system of E132, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 1.
E141. The two-vector system of E132 or E140, wherein the STRC promoter consists of SEQ ID NO: 1.
E142. The two-vector system of E132, wherein the STRC promoter has at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 2 or a functional portion thereof comprising nucleotides 252-537 or 35-530 of SEQ ID NO: 2.
E143. The two-vector system of E132 or E142, wherein the functional portion of SEQ ID NO: 2 comprises or consists of nucleotides 252-537 of SEQ ID NO: 2.
E144. The two-vector system of E132 or E142, wherein the functional portion of SEQ ID NO: 2 comprises or consists of nucleotides 120-537 of SEQ ID NO: 2.
E145. The two-vector system of E132 or E142, wherein the functional portion of SEQ ID NO: 2 comprises or consists of nucleotides 35-530 of SEQ ID NO: 2.
E146. The two-vector system of E132 or E142, wherein the STRC promoter consists of SEQ ID NO: 2.
E147. The two-vector system of any one of E108-E123 and E131-E146, wherein the first nucleic acid vector further comprises a polynucleotide encoding an N-terminal intein (N-intein) positioned 3′ of the first polynucleotide.
E148. The two-vector system of any one of E131-E147, wherein the second nucleic acid vector further comprises a polynucleotide encoding a C-terminal intein (C-intein) positioned between the STRC promoter and the second polynucleotide.
E149. The two-vector system of E148, wherein the N-intein and C-intein are components of a split intein trans-splicing system.
E150. The two-vector system of E149, wherein the split intein trans-splicing system is derived from a DnaE gene of one or more bacteria.
E151. The two-vector system of E149, wherein the N-intein has a sequence of any one of SEQ ID NOs: 7, 9, 12, 14, 16-21, 26, 28, 30, 32, 34, 36, 38, 49, 51, 53, 55, and 57 and the C-intein has a sequence of any one of SEQ ID NOs: 8, 10, 11, 13, 15, 22-25, 27, 29, 31, 33, 35, 37, 39, 50, 52, 54, 56, and 58.
E152. The two-vector system of E149, wherein the N-intein has the sequence of SEQ ID NO: 7 and the C-intein has the sequence of SEQ ID NO: 8.
E153. The two-vector system of E149, wherein N-intein has the sequence of SEQ ID NO: 7 and the C-intein has the sequence of SEQ ID NO: 10.
E154. The two-vector system of E149, wherein the N-intein has the sequence of SEQ ID NO: 7 and the C-intein has the sequence of SEQ ID NO: 11.
E155. The two-vector system of E149, wherein the N-intein has the sequence of SEQ ID NO: 9 and the C-intein has the sequence of SEQ ID NO: 8.
E156. The two-vector system of E149, wherein the N-intein has the sequence of SEQ ID NO: 9 and the C-intein has the sequence of SEQ ID NO: 10.
E157. The two-vector system of E149, wherein the N-intein has the sequence of SEQ ID NO: 9 and the C-intein has the sequence of SEQ ID NO: 11.
E158. The two-vector system of E149, wherein the N-intein has the sequence of SEQ ID NO: 12 and the C-intein has the sequence of SEQ ID NO: 13.
E159. The two-vector system of E149, wherein the N-intein has the sequence of SEQ ID NO: 14 and the C-intein has the sequence of SEQ ID NO: 15.
E160. The two-vector system of E149, wherein the N-intein has the sequence of SEQ ID NO: 16 and the C-intein has the sequence of SEQ ID NO: 22.
E161. The two-vector system of E149, wherein the N-intein has the sequence of SEQ ID NO: 19 and the C-intein has the sequence of SEQ ID NO: 23.
E162. The two-vector system of E149, wherein the N-intein has the sequence of SEQ ID NO: 20 and the C-intein has the sequence of SEQ ID NO: 24.
E163. The two-vector system of E149, wherein the N-intein has the sequence of SEQ ID NO: 21 and the C-intein has the sequence of SEQ ID NO: 25.
E164. The two-vector system of E149, wherein the N-intein has the sequence of SEQ ID NO: 26 and the C-intein has the sequence of SEQ ID NO: 27.
E165. The two-vector system of E149, wherein the N-intein has the sequence of SEQ ID NO: 28 and the C-intein has the sequence of SEQ ID NO: 29.
E166. The two-vector system of E149, wherein the N-intein has the sequence of SEQ ID NO: 30 and the C-intein has the sequence of SEQ ID NO: 31.
E167. The two-vector system of E149, wherein the N-intein has the sequence of SEQ ID NO: 32 and the C-intein has the sequence of SEQ ID NO: 33.
E168. The two-vector system of E149, wherein the N-intein has the sequence of SEQ ID NO: 34 and the C-intein has the sequence of SEQ ID NO: 35.
E169. The two-vector system of E149, wherein the N-intein has the sequence of SEQ ID NO: 36 and the C-intein has the sequence of SEQ ID NO: 37.
E170. The two-vector system of E149, wherein the N-intein has the sequence of SEQ ID NO: 38 and the C-intein has the sequence of SEQ ID NO: 39.
E171. The two-vector system of E149, wherein the N-intein has the sequence of any one of SEQ ID NOs: 16-21 and the C-intein has the sequence of any one of SEQ ID NOs: 22-25.
E172. The two-vector system of E149, wherein the N-intein has the sequence of SEQ ID NO: 49 and the C-intein has the sequence of SEQ ID NO: 50.
E173. The two-vector system of E149, wherein the N-intein has the sequence of SEQ ID NO: 51 and the C-intein has the sequence of SEQ ID NO: 52.
E174. The two-vector system of E149, wherein the N-intein has the sequence of SEQ ID NO: 53 and the C-intein has the sequence of SEQ ID NO: 54.
E175. The two-vector system of E149, wherein the N-intein has the sequence of SEQ ID NO: 55 and the C-intein has the sequence of SEQ ID NO: 56.
E176. The two-vector system of E149, wherein the N-intein has the sequence of SEQ ID NO: 57 and the C-intein has the sequence of SEQ ID NO: 58.
E177. The two-vector system of any one of E108-E176, wherein the two-vector system directs hair cell-specific expression of a full-length stereocilin protein in a mammalian hair cell.
E178. The two-vector system of E177, wherein the hair cell is a cochlear hair cell.
E179. The two-vector system of E178, wherein the cochlear hair cell is an outer hair cell.
E180. The two-vector system of E178 wherein the cochlear hair cell is an inner hair cell.
E181. The two-vector system of E177, wherein the hair cell is a vestibular hair cell.
E182. The two-vector system of E181, wherein the vestibular hair cell is a Type I vestibular hair cell.
E183. The two-vector system of E181, wherein the vestibular hair cell is a Type II vestibular hair cell.
E184. The two-vector system of any one of E177-E183, wherein the mammalian hair cell is a human hair cell.
E185. The two-vector system of any one of E108-E184, wherein the stereocilin protein is a human stereocilin protein having at least 85% sequence identity (e.g., 85%, 86%, 87%, 88%, 89%, 90%, 91%, 92%, 93%, 94%, 95%, 96%, 97%, 98%, 99%, or more, sequence identity) to SEQ ID NO: 3.
E186. The two-vector system of any one of E108-E185, wherein the first and second vectors are viral vectors, plasmids, cosmids, or artificial chromosomes.
E187. The two-vector system of E186, wherein the first and second vectors are viral vectors selected from the group consisting of AAV vectors, adenovirus vectors, and lentivirus vectors.
E188. The two-vector system of E187, wherein the first and second vectors are AAV vectors.
E189. The two-vector system of E188, wherein each of the AAV vectors has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S capsid.
E190. A composition comprising the two-vector system of any one of E108-E189.
E191. The composition of E190, further comprising a pharmaceutically acceptable carrier, diluent, or excipient.
E192. A cell comprising the two-vector system of any one of E108-E189.
E193. The cell of E192, wherein the cell is a hair cell.
E194. The cell of E193, wherein the hair cell is a mammalian hair cell.
E195. The cell of E194, wherein the mammalian hair cell is a human hair cell.
E196. The cell of any one of E192-E195, wherein the hair cell is a cochlear hair cell.
E197. The cell of E196, wherein the cochlear hair cell is an outer hair cell.
E198. The cell of E196, wherein the cochlear hair cell is an inner hair cell.
E199. The cell of any one of E192-E195, wherein the hair cell is a vestibular hair cell.
E200. The cell of E199, wherein the vestibular hair cell is a type II vestibular hair cell.
E201. The cell of E199, wherein the vestibular hair cell is a type I vestibular hair cell.
E202. A method of expressing a stereocilin protein in a hair cell, comprising contacting the hair cell with the two-vector system of any one of E108-E189 or the composition of E190 or E191.
E203. The method of E202, wherein the contacting is in vivo (e.g., in a subject).
E204. The method of E202, wherein the stereocilin protein is specifically expressed in hair cells.
E205. A method of treating a subject having or at risk of developing sensorineural hearing loss (e.g., nonsyndromic hearing loss), comprising administering to an inner ear of the subject an effective amount of the two-vector system of any one of E108-E189 or the composition of E190 or E191.
E206. A method of treating a subject having or at risk of developing vestibular dysfunction, comprising administering to an inner ear of the subject an effective amount of the two-vector system of any one of E108-E189 or the composition of E190 or E191.
E207. A method of increasing hair cell survival in a subject in need thereof, comprising administering to an inner ear of the subject an effective amount of the two-vector system of any one of E108-E189 or the composition of E190 or E191.
E208. A method of preventing or reducing hair cell damage or death in a subject in need thereof, comprising administering to an inner ear of the subject an effective amount of the two-vector system of any one of E108-E189 or the composition of E190 or E191.
E209. A method of improving hair cell function in a subject in need thereof, comprising administering to an inner ear of the subject an effective amount of the nucleic acid vector of any one of E19-E44, two-vector system of any one of E108-E189, or the composition of E45, E46, E190, or E191.
E210. The method of any one of E202-E204 and E207-E209, wherein the hair cell is a mammalian hair cell.
E211. The method of E210, wherein the mammalian hair cell is a human hair cell.
E212. The method of any one of E202-E204 and E207-E211, wherein the hair cell is a cochlear hair cell.
E213. The method of E212, wherein the cochlear hair cell is an outer hair cell.
E214. The method of E212, wherein the cochlear hair cell is an inner hair cell.
E215. The method of any one of E202-E204 and E207-E211, wherein the hair cell is a vestibular hair cell.
E216. The method of E215, wherein the vestibular hair cell is a type II vestibular hair cell.
E217. The method of E215, wherein the vestibular hair cell is a type I vestibular hair cell.
E218. A method of increasing STRC expression (expressing STRC) in a subject in need thereof, the method comprising administering to an inner ear of the subject a therapeutically effective amount of the two-vector system of any one of E108-E189 or the composition of E190 or E191.
E219. The method of any one of E207-E214 and E218, wherein the subject has or is at risk of developing hearing loss (e.g., sensorineural hearing loss).
E220. The method of E205 or E219, wherein the hearing loss is genetic hearing loss.
E221. The method of E220, wherein the genetic hearing loss is autosomal recessive hearing loss.
E222. The method of any one of E207-E211 and E215-E218, wherein the subject has or is at risk of developing vestibular dysfunction.
E223. The method of E206 or E222, wherein the vestibular dysfunction comprises vertigo, dizziness, imbalance, bilateral vestibulopathy, oscillopsia, or a balance disorder.
E224. The method of any one of E206, E222, and E223, wherein the vestibular dysfunction is associated with a genetic mutation.
E225. The method of any one of E205-E224, wherein the subject has a mutation in STRC.
E226. The method of any one of E205-E225, wherein the subject has been identified as having a mutation in STRC.
E227. The method of any one of E205-E225, wherein the method further comprises identifying the subject as having a mutation in STRC prior to administering the two-vector system or composition.
E228. The method of any one of E205-E227, wherein the subject has DFNB16.
E229. The method of any one of E205-E226, wherein the subject has been identified as having DFNB16.
E230. The method of any one of E205-E229, wherein the method further comprises evaluating the hearing of the subject prior to administering the nucleic acid vector or composition.
E231. The method of any one of E205-E230, wherein the method further comprises evaluating the hearing of the subject after administering the nucleic acid vector or composition.
E232. The method of any one of E205-E231, wherein the method further comprises evaluating the vestibular function of the subject prior to administering the nucleic acid vector or composition.
E233. The method of any one of E205-E232, wherein the method further comprises evaluating the vestibular function of the subject after administering the nucleic acid vector or composition.
E234. The method of any one of E205-E233, wherein the nucleic acid vector or composition is administered locally to the inner ear.
E235. The method of E234, wherein the nucleic acid vector or composition is administered to a semicircular canal.
E236. The method of E234, wherein the nucleic acid vector or composition is administered transtympanically or intratympanically.
E237. The method of E234, wherein the nucleic acid vector or composition is administered into the perilymph.
E238. The method of E234, wherein the nucleic acid vector or composition is administered into the endolymph.
E239. The method of E234, wherein the nucleic acid vector or composition is administered to or through the oval window.
E240. The method of E234, wherein the nucleic acid vector or composition is administered to or through the round window.
E241. The method of any one of E205-E240, wherein the vectors in the two-vector system are administered concurrently.
E242. The method of any one of E205-E240, wherein the vectors in the two-vector system are administered sequentially.
E243. The method of any one of E205-E242, wherein the two-vector system or pharmaceutical composition is administered in an amount sufficient to prevent or reduce hearing loss, delay the development of hearing loss, slow the progression of hearing loss, improve hearing, improve speech discrimination, prevent or reduce vestibular dysfunction, delay the development of vestibular dysfunction, slow the progression of vestibular dysfunction, improve vestibular function, improve hair cell function, prevent or reduce hair cell damage, prevent or reduce hair cell death, promote or increase hair cell survival, or increase STRC expression in a hair cell.
E244. The method of any one of E205-E243, wherein the subject is a human subject.
E245. A kit comprising the polynucleotide of any one of E1-E18, the nucleic acid vector of any one of E19-E44, the two-vector system of any one of E108-E189, or the composition of E45, E46, E190, or E191.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A polynucleotide comprising a STRC promoter having at least 85% sequence identity to SEQ ID NO: 48 or a functional portion thereof comprising nucleotides 280-560 of SEQ ID NO: 48 operably linked to a polynucleotide encoding a heterologous expression product.

2. A polynucleotide comprising a STRC promoter having: (i) at least 85% sequence identity to SEQ ID NO: 1; or (ii) at least 85% sequence identity to SEQ ID NO: 2 or a functional portion thereof comprising nucleotides 252-537 or 35-530 of SEQ ID NO: 2, operably linked to a polynucleotide encoding a heterologous expression product.

3. The polynucleotide of claim 1, wherein the STRC promoter has at least 85% sequence identity to SEQ ID NO: 48.

4. The polynucleotide of claim 1 or 3, wherein the STRC promoter has the sequence of SEQ ID NO: 48.

5. The polynucleotide of claim 1, wherein the functional portion of SEQ ID NO: 48 comprises nucleotides 280-560 of SEQ ID NO: 48.

6. The polynucleotide of claim 1, wherein the functional portion of SEQ ID NO: 48 comprises nucleotides 280-564 of SEQ ID NO: 48.

7. The polynucleotide of claim 1, wherein the functional portion of SEQ ID NO: 48 comprises nucleotides 124-560 of SEQ ID NO: 48.

8. The polynucleotide of claim 1, wherein the functional portion of SEQ ID NO: 48 comprises nucleotides 124-564 of SEQ ID NO: 48.

9. The polynucleotide of claim 1, wherein the functional portion of SEQ ID NO: 48 comprises nucleotides 1-560 of SEQ ID NO: 48.

10. The polynucleotide of claim 2, wherein the STRC promoter has at least 85% sequence identity to SEQ ID NO: 1.

11. The polynucleotide of claim 2 or 10, wherein the STRC promoter has the sequence of SEQ ID NO: 1.

12. The polynucleotide of claim 2, wherein the STRC promoter has at least 85% sequence identity to SEQ ID NO: 2 or a functional portion thereof comprising nucleotides 252-537 or 35-530 of SEQ ID NO: 2.

13. The polynucleotide of claim 2 or 12, wherein the functional portion of SEQ ID NO: 2 comprises nucleotides 252-537 of SEQ ID NO: 2.

14. The polynucleotide of claim 13, wherein the functional portion of SEQ ID NO: 2 comprises nucleotides 120-537 of SEQ ID NO: 2.

15. The polynucleotide of claim 2 or 12, wherein the functional portion of SEQ ID NO: 2 comprises nucleotides 35-530 of SEQ ID NO: 2.

16. The polynucleotide of claim 2 or 12, wherein the STRC promoter has the sequence of SEQ ID NO: 2.

17. The polynucleotide of any one of claims 1-16, wherein the heterologous expression product is a protein, a short hairpin RNA (shRNA), an antisense oligonucleotide (ASO), a component of a gene editing system, or a microRNA.

18. The polynucleotide of claim 17, wherein the protein is Actin Gamma 1 (ACTG1), Fascin Actin-Bundling Protein 2, Retinal (FSCN2), Radixin (RDX), POU Class 4 Homeobox 3 (POU4F3), TRIO and F-Actin Binding Protein (TRIOBP), Taperin (TPRN), Xin Actin Binding Repeat Containing 2 (XIRP2), Atonal BHLH Transcription Factor 1 (ATOH1), Growth Factor Independent 1 Transcriptional Repressor (GFI1), Cholinergic Receptor Nicotinic Alpha 9 Subunit (CHRNA9), Cholinergic Receptor Nicotinic Alpha 10 Subunit (CHRNA10), Calcium and Integrin Binding Family Member 3 (CIB3), Cadherin 23 (CDH23), Protocadherin 15 (PCDH15), Kinocilin (KNCN), Pejvakin (DFNB59), MKRN2 Opposite Strand (MKRN2OS), LIM Homeobox Protein 3 (LHX3), Transmembrane Channel Like 1 (TMC1), Myosin 15 (MYO15), Myosin 7A (MYO7A), Myosin 6 (MYO6), Myosin IIIA (MYO3A), Myosin IIIB (MYO3B), Glutaredoxin Domain Containing Cysteine-Rich Protein 1 (GRXCR1), Protein Tyrosine Phosphatase, Receptor Type Q (PTPRQ), Late Cornified Envelope 6A (LCE6A), Lipoxygenase Homology Domain-containing Protein 1 (LOXHD1), ADP-Ribosyltransferase 1 (ART1), ATPase Plasma Membrane Ca2+ Transporting 2 (ATP2B2), Calcium and Integrin Binding Family Member 2 (CIB2), Calcium Voltage-Gated Channel Auxiliary Subunit Alpha2delta 4 (CACNA2D4), Epidermal Growth Factor Receptor Pathway Substrate 8 (EPS8), EPS8 Like 2 (EPS8L2), Espin (ESPN), Espin Like (ESPNL), Peripherin 2 (PRPH2), Solute Carrier Family 8 Member A2 (SLC8A2), Zinc Finger CCHC-Type Containing Protein 12 (ZCCHC12), Leucine Rich Transmembrane and O-methyltransferase Domain Containing (LRTOMT2, LRTOMT1), USH1 Protein Network Component Harmonin (USH1C), Solute Carrier Family 26 Member 5 (SLC26A5), Piezo Type Mechanosensitive Ion Channel Component 2 (PIEZO2), Extracellular Leucine Rich Repeat and Fibronectin Type III Domain Containing 1 (ELFN1), Tetratricopeptide Repeat Protein 24 (TTC24), Dystrotelin (DYTN), Coiled-coil Glutamate Rich Protein 2 (CCER2), Leucine-rich Repeat and Transmembrane Domain-containing protein 2 (LRTM2), Potassium Voltage-Gated Channel Subfamily A Member 10 (KCNA10), Clarin 1 (CLRN1), Clarin 2 (CLRN2), SKI Family Transcriptional Corepressor 1 (SKOR1), Tctexl Domain Containing Protein 1 (TCTEX1 D1), Fc Receptor Like B (FCRLB), Glutaredoxin Domain Containing Cysteine-Rich Protein 2 (GRXCR2), Serpin Family E Member 3 (SERPINE3), Nescient Helix-loop Helix 1 (NHLH1), Heat Shock Protein 70 (HSP70), Heat Shock Protein 90 (HSP90), Activating Transcription Factor 6 (ATF6), Eukaryotic Translation Initiation Factor 2 Alpha Kinase 3 (PERK), Serine/Threonine-Protein Kinase/Endoribonuclease IRE1 (IRE1), Whirlin (WHRN), Oncomodulin (OCM), LIM Homeobox 1 (Isl1), Transmembrane and Tetratricopeptide Repeat Containing 4 (TMTC4), Binding Immunoglobulin Protein (BIP), or Potassium Voltage-Gated Channel Subfamily Q Member 4 (KCNQ4).

19. A nucleic acid vector comprising the polynucleotide of any one of claims 1-18.

20. The nucleic acid vector of claim 19, wherein the nucleic acid vector is a viral vector.

21. The nucleic acid vector of claim 20, wherein the viral vector is an adeno-associated virus (AAV) vector.

22. A two-vector system comprising:
a) a first nucleic acid vector comprising a STRC promoter having at least 85% sequence identity to SEQ ID NO: 48 or a functional portion thereof comprising nucleotides 280-560 of SEQ ID NO: 48 operably linked to a first polynucleotide encoding an N-terminal portion of a stereocilin protein; and
b) a second nucleic acid vector comprising a second polynucleotide encoding a C-terminal portion of a stereocilin protein.

23. A two-vector system comprising:
a) a first nucleic acid vector comprising a STRC promoter having: (i) at least 85% sequence identity to SEQ ID NO: 1; or (ii) at least 85% sequence identity to SEQ ID NO: 2 or a functional portion thereof comprising nucleotides 252-537 or 35-530 of SEQ ID NO: 2, operably linked to a first polynucleotide encoding an N-terminal portion of a stereocilin protein; and
b) a second nucleic acid vector comprising a second polynucleotide encoding a C-terminal portion of a stereocilin protein.

24. The two-vector system of claim 22, wherein the STRC promoter has at least 85% sequence identity to SEQ ID NO: 48.

25. The two-vector system of claim 22 or 24, wherein the STRC promoter has the sequence of SEQ ID NO: 48.

26. The two-vector system of claim 22, wherein the functional portion of SEQ ID NO: 48 comprises nucleotides 280-560 of SEQ ID NO: 48.

27. The two-vector system of claim 22, wherein the functional portion of SEQ ID NO: 48 comprises nucleotides 280-564 of SEQ ID NO: 48.

28. The two-vector system of claim 22, wherein the functional portion of SEQ ID NO: 48 comprises nucleotides 124-560 of SEQ ID NO: 48.

29. The two-vector system of claim 22, wherein the functional portion of SEQ ID NO: 48 comprises nucleotides 124-564 of SEQ ID NO: 48.

30. The two-vector system of claim 22, wherein the functional portion of SEQ ID NO: 48 comprises nucleotides 1-560 of SEQ ID NO: 48.

31. The two-vector system of claim 23, wherein the STRC promoter has at least 85% sequence identity to SEQ ID NO: 1.

32. The two-vector system of claim 23 or 31, wherein the STRC promoter has the sequence of SEQ ID NO: 1.

33. The two-vector system of claim 23, wherein the STRC promoter has at least 85% sequence identity to SEQ ID NO: 2 or a functional portion thereof comprising nucleotides 252-537 or 35-530 of SEQ ID NO: 2.

34. The two-vector system of claim 33, wherein the functional portion of SEQ ID NO: 2 comprises nucleotides 252-537 of SEQ ID NO: 2.

35. The two-vector system of claim 34, wherein the functional portion of SEQ ID NO: 2 comprises nucleotides 120-537 of SEQ ID NO: 2.

36. The two-vector system of claim 23 or 33, wherein the functional portion of SEQ ID NO: 2 comprises nucleotides 35-530 of SEQ ID NO: 2.

37. The two-vector system of claim 23 or 33, wherein the STRC promoter has the sequence of SEQ ID NO: 2.

38. The two-vector system of any one of claims 22-37, wherein the first polynucleotide partially overlaps with the second polynucleotide.

39. The two-vector system of any one of claims 22-38, wherein the first polynucleotide and the second polynucleotide have a region of overlap having a length of at least 200 bases (b).

40. The two-vector system of any one of claims 22-39, wherein when introduced into a mammalian cell, the first and second nucleic acid vectors undergo homologous recombination to form a recombined polynucleotide that encodes a full-length stereocilin protein.

41. The two-vector system of any one of claims 22-37, wherein
the first nucleic acid vector comprises a splice donor signal sequence positioned 3′ of the first polynucleotide; and
the second nucleic acid vector comprises a splice acceptor signal sequence positioned 5′ of the second polynucleotide.

42. The two-vector system of any one of claims 22-37, wherein
the first nucleic acid vector comprises a splice donor signal sequence positioned 3′ of the first polynucleotide and a first recombinogenic region positioned 3′ of the splice donor signal sequence; and
the second nucleic acid vector comprises a second recombinogenic region and a splice acceptor signal sequence positioned 3′ of the recombinogenic region and 5′ of the second polynucleotide.

43. The two-vector system of any one of claims 22-37, 41, and 42, wherein the first and second polynucleotides do not overlap.

44. The two-vector system of claim 42 or 43, wherein
the first nucleic acid vector further comprises a degradation signal sequence positioned 3′ of the recombinogenic region; and
the second nucleic acid vector further comprises a degradation signal sequence positioned between the recombinogenic region and the splice acceptor signal sequence.

45. The two-vector system of any one of claims 22-37, wherein the second nucleic acid vector further comprises a STRC promoter having at least 85% sequence identity to SEQ ID NO: 48 or a functional portion thereof comprising nucleotides 280-560 of SEQ ID NO: 48 operably linked to the second polynucleotide, wherein the STRC promoter is positioned 5′ of the second polynucleotide.

46. The two-vector system of any one of claims 22-37, wherein the second nucleic acid vector further comprises a STRC promoter having: (i) at least 85% sequence identity to SEQ ID NO: 1; or (ii) at least 85% sequence identity to SEQ ID NO: 2 or a functional portion thereof comprising nucleotides 252-537 or 35-530 of SEQ ID NO: 2, operably linked to the second polynucleotide, wherein the STRC promoter is positioned 5′ of the second polynucleotide.

47. The two-vector system of any one of claims 22-37, 45, and 46, wherein the first nucleic acid vector further comprises a polynucleotide encoding an N-terminal intein (N-intein) positioned 3′ of the first polynucleotide.

48. The two-vector system of any one of claims 45-47, wherein the second nucleic acid vector further comprises a polynucleotide encoding a C-terminal intein (C-intein) positioned between the STRC promoter and the second polynucleotide.

49. The two-vector system of claim 48, wherein the N-intein and C-intein are components of a split intein trans-splicing system.

50. The two-vector system of any one of claims 22-49, wherein the first polynucleotide encoding the N-terminal portion of a stereocilin protein and the second polynucleotide encoding the C-terminal portion of a stereocilin protein do not comprise introns.

51. The two-vector system of any one of claims 22-50, wherein the two-vector system directs hair cell-specific expression of a full-length stereocilin protein in a mammalian hair cell.

52. The two-vector system of any one of claims 22-51, wherein the stereocilin protein is a human stereocilin protein having at least 85% sequence identity to SEQ ID NO: 3.

53. The two-vector system of any one of claims 22-52, wherein the first and second vectors are adeno-associated virus (AAV) vectors.

54. A composition comprising the nucleic acid vector of any one of claims 19-21 or the two-vector system of any one of claims 22-53 and a pharmaceutically acceptable carrier, diluent, or excipient.

55. A method of expressing a heterologous expression product in a hair cell, comprising contacting the hair cell with the nucleic acid vector of any one of claims 19-21.

56. A method of expressing a stereocilin protein in a human hair cell, comprising contacting the human hair cell with the two-vector system of any one of claims 22-53.

57. The method of claim 55 or 56, wherein the contacting is in a subject.

58. A method of treating a subject having or at risk of developing sensorineural hearing loss, comprising administering to an inner ear of the subject an effective amount of the nucleic acid vector of any one of claims 19-21, the two-vector system of any one of claims 22-53, or the composition of claim 54.

59. A method of treating a subject having or at risk of developing tinnitus, comprising administering to an inner ear of the subject an effective amount of the nucleic acid vector of any one of claims 19-21 or the composition of claim 54.

60. A method of treating a subject having or at risk of developing vestibular dysfunction, comprising administering to an inner ear of the subject an effective amount of the nucleic acid vector of any one of claims 19-21, the two-vector system of any one of claims 22-53, or the composition of claim 54.

61. A method of inducing or increasing hair cell regeneration in a subject in need thereof, comprising administering to an inner ear of the subject an effective amount of the nucleic acid vector of any one of claims 19-21 or the composition of claim 54.

62. A method of inducing or increasing hair cell maturation in a subject in need thereof, comprising administering to an inner ear of the subject an effective amount of the nucleic acid vector of any one of claims 19-21 or the composition of claim 54.

63. A method of preventing or reducing hair cell damage or death in a subject in need thereof, comprising administering to an inner ear of the subject an effective amount of the nucleic acid vector of any one of claims 19-21, the two-vector system of any one of claims 22-53, or the composition of claim 54.

64. A method of increasing hair cell survival in a subject in need thereof, comprising administering to an inner ear of the subject an effective amount of the nucleic acid vector of any one of claims 19-21, the two-vector system of any one of claims 22-53, or the composition of claim 54.

65. A method of improving hair cell function in a subject in need thereof, comprising administering to an inner ear of the subject an effective amount of the nucleic acid vector of any one of claims 19-21, the two-vector system of any one of claims 22-53, or the composition of claim 54.

66. A method of increasing STRC expression in a subject in need thereof, the method comprising administering to an inner ear of the subject a therapeutically effective amount of the two-vector system of any one of claims 22-53 or the composition of claim 54.

67. The method of any one of claims 61-66, wherein the subject has or is at risk of developing hearing loss.

68. The method of any one of claims 61-66, wherein the subject has or is at risk of developing vestibular dysfunction.

69. The method of any one of claims 58, 60, and 63-68, wherein the subject has a mutation in STRC.

70. The method of any one of claims 58, 60, and 63-69, wherein the subject has been identified as having a mutation in STRC.

71. The method of any one of claims 58, 60, and 63-69, wherein the method further comprises identifying the subject as having a mutation in STRC prior to administering the two-vector system or composition.

72. The method of any one of claims 58, 60, and 63-70, wherein the subject has deafness, autosomal recessive 16 (DFNB16).

73. The method of any one of claims 58-72, wherein the nucleic acid vector, the two-vector system, or the composition is administered locally to the inner ear.

74. A kit comprising the polynucleotide of any one of claims 1-18, the nucleic acid vector of any one of claims 19-21, the two-vector system of any one of claims 22-53, or the composition of claim 54.
